(19) United States
(12) Patent Application Publication
(10) Pub. No.: US 2024/0277396 A1
(43) Pub. Date: Aug. 22, 2024

(54) Minimally Invasive Diagnostic and Therapeutic Excision of Tissue

(71) Applicant: Eggers & Associates, LLC, Dublin, OH (US)

(72) Inventors: Philip E. Eggers, Dublin, OH (US); Andrew Eggers, Ostrander, OH (US)

(73) Assignee: Eggers & Associates, LLC, Dublin, OH (US)

(21) Appl. No.: 18/602,076

(22) Filed: Mar. 12, 2024

Related U.S. Application Data
(63) Continuation-in-part of application No. 15/877,730, filed on Jan. 23, 2018, now Pat. No. 11,737,808.

(51) Int. Cl. A61B 18/08; A61B 17/00; A61B 18/00

(52) U.S. Cl. CPC A61B 18/082; A61B 2017/00199; A61B 2017/00734; A61B 2018/00083; A61B 2018/00095; A61B 2018/00107; A61B 2018/00267; A61B 2018/00333; A61B 2018/00601; A61B 2018/0072; A61B 2018/00875; A61B 2018/0091

(57) ABSTRACT

The recovery of an intact volume of tissue proceeds with a delivery cannula distal end positioned in confronting adjacency with the volume of tissue to be recovered. A tissue cutting and capture assembly formed of a plurality of metal leafs is deployed from the distal end of the delivery cannula. The tips of these leafs carry a pursing cable assembly, which is electrically excited to electrosurgically cut around and circumscribe the tissue volume. These pursing cables are tensioned to complete the envelopment of the tissue volumes by drawing the leaf tips together. An essential attribute of the disclosed apparatus is the confinement of the path of electrical conduction of constant current required to achieve tissue cutting to only those portions of the deploying and retracting resistively heated portion of the electrically conductive cutting and pursing cable that are in direct contact with tissue.

### CROSS-REFERENCE TO RELATED APPLICATIONS

This application is a continuation-in-part of U.S. Ser. No. 15/877,730 filed Jan. 23, 2018; and is cross-referenced to U.S. Pat. No. 11,737,808.

### STATEMENT REGARDING FEDERALLY SPONSORED RESEARCH

Not applicable.

### THE NAMES OF THE PARTIES TO A JOINT RESEARCH AGREEMENT

Not applicable.

### REFERENCE TO A “SEQUENCE LISTING,” A TABLE, OR A COMPUTER PROGRAM

Not applicable.

### STATEMENT REGARDING PRIOR DISCLOSURES BY THE INVENTOR OR A JOINT INVENTOR

Not applicable.

### BACKGROUND OF THE INVENTION

It is estimated that one out of eight women will face breast cancer at some point during their lifetime, and for women age 40-55, breast cancer is the leading cause of death. While methods for detecting and treating breast cancer initially were crude and unsophisticated, advanced instrumentation and procedures now are available which provide more positive outcomes for patients.

In the 1800s the only treatment for breast cancer was removal of the entire breast. Given that the sole method of detection and diagnosis was palpation, treatment was only directed when the breast tumor was well advanced. Modified radical mastectomies are still performed today for patients with invasive cancer, such a procedure involving the removal of the entire breast and some or all of the axillary lymph nodes. Radical or modified radical mastectomies involve serious trauma for the patient during surgery, with the severest cosmetic results after surgery.

Another surgical option upon the discovery of malignant tumor is what is referred to as breast conserving surgery, which also is referred to as lumpectomy, tumorectomy, segmental mastectomy or local excision. Meant to address the cosmetic concerns associated with removal of the breast, only the primary tumor and a margin of surrounding normal breast tissue is removed. Determining the proper amount of tissue to be removed involves balancing the need to take sufficient tissue to prevent recurrence with the desire to take as little tissue as possible to preserve the best cosmetic appearance. A more limited nodal dissection now is performed with the primary purpose being staging rather than therapy. While an improvement over radical mastectomy, breast-conserving surgery still involves the removal of large sections of breast tissue. Risks associated with such surgery include wound infection, seroma formation, mild shoulder dysfunction, loss of sensation in the distribution of the intercostobrachial nerve, and edema of the breast and arm. For more information on invasive tumor therapy, see:
(1) Harris, Jay R., et al. “Cancer of the Breast.” Cancer: Principles and Practices of Oncology, Fourth Edition. Eds. DeVita, et al. Philadelphia: J. B. Lippincott Co., 1993. 1264-1285.
(2) Jobe, William E. “Historical Perspectives.” Percutaneous Breast Biopsy. Eds. Parker, et al. New York: Raven Press, 1993. 1-5.

Mastectomies and breast-conserving surgeries generally are procedures utilized for invasive tumors. Advances in tumor detection, however, have radically changed the course of diagnosis and treatment for a tumor. With the advent of imaging devices, such as the mammogram, a suspect tumor may be located when it is of relatively small size. Today, tumor detection generally involves both a mammogram and a physical examination, which takes into account a number of risk factors including family history and prior occurrences. Technical improvements in mammogram imaging include better visualization of the breast parenchyma with less exposure to radiation, improvements in film quality and processing, improved techniques for imaging, better guidelines for the diagnosis of cancer and greater availability of well-trained mammographers. With these advances in imaging technology, a suspect tumor may be detected which is 5 mm or smaller. More recently substantial progress has been witnessed in the technical disciplines of magnetic resonance imaging (MRI) and ultrasound imagining. With these advances, the location of a lesion is observable as diagnostic or therapeutic procedures are carried out.

In the past, because a tumor normally was not discovered until it had reached an advanced stage, the issue of whether a tumor was malignant or benign did not need to be addressed. With the ability to locate smaller areas of suspect tumor, this issue becomes of critical importance, particularly in light of the fact that only 20% of small, non-invasive tumors are malignant. Tumors identified as being benign may be left in situ with no excision required, whereas action must be taken to excise suspect tissue confirmed to be malignant. In view of the value of classifying a tumor as malignant or benign, breast biopsy has become a much-utilized technique with over 1 million biopsies being performed annually in the United States. A biopsy procedure involves the two-step process of first locating the tumor then removing part or all of the suspect tissue for examination to establish precise diagnosis.

Improvements in the detection of suspicious lesions in the breast are described in U.S. Patent Publication No. US 2006/0036173 published Feb. 16, 2006. In this patent application ultrasonic scanning and diagnostics for cellular tissue are disclosed. An ultrasonic tissue incision and retrieval assembly is moved across cellular tissue at a rate that is synchronized with the image capture rate of the ultrasonic scanner, to achieve a contiguous and complete set of scan images of the tissue. The tissue incision and retrieval assembly can be held in a single position as it is moved across the tissue, or it can be dynamically adjusted during the scan to provide optimal contact with the scanned tissue. The image data are captured and converted to a format that is easily stored and compatible with a viewer. The viewer allows playback of the scanned images in a manner that is optimized for screening for cancers and other anomalies. A location function allows the user to select a point of interest on an individual scan image, and choose another known reference point, and the function calculates and provides the distance from the reference point to the point of interest in three dimensions. The system can be used for virtually any tissue, but can also be optimized for breast cancer screening. Clinical studies using the method and apparatus described in this patent application have revealed that suspicious and potentially malignant lesions in the human breast can be detected having maximum dimensional extents as small as 2 to 3 mm. This non-invasive diagnostic imaging capability would enable the complete excision of such small lesions surrounded by healthy margins of tissue in volumes as small as 10 to 15 mm in diameter using minimally invasive excisional methods.

One biopsy option available upon detection of a suspected tumor is an open surgical biopsy or excisional biopsy. Prior to surgery, a radiologist, using mammography, inserts a wire into the breast to locate the suspected tumor site. Later during surgery, the surgeon makes an incision in the breast and removes a large section of breast tissue, including the suspect tissue and a margin of healthy tissue surrounding the tumor. As with other similar procedures, such as those described above, open surgery may result in high levels of blood loss, scarring at the location of the incision and permanent disfigurement, due to the removal of relatively large amounts of tissue. Because of the critical prognostic significance of tumor size, the greatest advantage of the excisional biopsy is that the entire area of the suspect tumor is removed. After being removed and measured, the specimen is typically transected by a pathologist in a plane that should bisect a suspected tumor and then the margin between tumor and healthy tissue is examined. Microscopic location of carcinoma near the margin provides information for future prognosis. Thus, the pathology laboratory is oriented to the morphological aspect of analysis, i.e. the forms and structures of involved tissue. For information on pathology of breast biopsy tissue, see:
(3) Rosen, Paul Peter. Rosen's Breast Pathology. Philadelphia: Lippincott-Raven Publishers, 1997. 837-858.

Other less invasive options are available which avoid the disadvantages associated with open surgery. One such non-invasive option is that of needle biopsy, which may be either fine needle aspiration or large core. Fine needle aspiration (FNA) is an office procedure in which a fine needle, for example of 21 to 23 gauge, having one of a number of tip configurations, such as the Chiba, Franzeen or Turner, is inserted into the breast and guided to the tumor site by mammography or stereotactic imaging. A vacuum is created and the needle moved up and down along the tumor to assure that it collects targeted cellular material. Generally, three or more passes will be made to assure the collection of a sufficient sample. The needle and the tissue sample are then withdrawn from the breast.

The resulting specimen is subject to a cytological assay, as opposed to the above-noted morphological approach. In this regard, cell structure and related aspects are studied. The resultant analysis has been used to improve or customize the selection of chemotherapeutic agents with respect to a particular patient. While a fine needle aspiration biopsy has the advantages of being a relatively simple and inexpensive office procedure, there are some drawbacks associated with its use. With fine needle aspiration, there is a risk of false-negative results, which most often occurs in cases involving extremely fibrotic tumor. In addition, after the procedure has been performed there may be insufficient specimen material for diagnosis. Finally, with fine needle aspiration alone the entire area of suspect tissue is not removed. Rather, fragmented portions of tissue are withdrawn which do not allow for the same type of pathological investigation as the tissue removed during an open surgery biopsy.

This last limitation also is observed with respect to large core needle biopsies. For a large core needle biopsy, a 14 to 18-gauge needle is inserted in the breast having an inner trocar with a sample notch at the distal end and an outer cutting cannula. Similar to a fine needle aspiration, tissue is drawn through the needle by vacuum suction. These needles have been combined with biopsy guns to provide automated insertion that makes the procedure shorter and partially eliminates location mistakes caused by human error. Once inserted, multiple contiguous tissue samples may be taken.

Samples taken during large core needle biopsies may be anywhere from friable and fragmented to large pieces 20 to 30 mm long. These samples may provide some histological data, unlike fine needle aspiration samples; however, they still do not provide the pathological information available with an open surgical biopsy specimen. Further, as with any mechanical cutting device, excessive bleeding may result during and following the procedure. Needle biopsy procedures are discussed in:
(4) Parker, Steve H. “Needle Selection” and “Stereotactic Large-Core Breast Biopsy.” Percutaneous Breast Biopsy. Eds. Parker, et al. New York: Raven Press, 1993. 7-14 and 61-79.

A device which is somewhere between a needle biopsy and open surgery is referred to as the Advanced Breast Biopsy Instrumentation (ABBI). With the ABBI procedure, the practitioner, guided by stereotactic imaging, removes a core tissue sample of 5 mm to 20 mm in diameter. While the ABBI has the advantage of providing a large tissue sample, similar to that obtained from an open surgical biopsy, the cylindrical tissue sample is taken from the subcutaneous tissue to an area beyond the suspect tumor. For tumors embedded more deeply within the breast, the amount of tissue removed is considerable. In addition, while less expensive than open surgical biopsy, the ABBI has proven expensive compared to other biopsy techniques, and it has been noted that the patient selection for the ABBI is limited by the size and location of the tumor, as well as by the presence of very dense parenchyma around the tumor. For discussion on the ABBI, see:
(5) Parker, Steve H. “The Advanced Breast Biopsy Instrumentation: Another Trojan Horse?” Am. J. Radiology 1998; 171: 51-53.
(6) D'Angelo, Philip C., et al. “Stereotactic Excisional Breast Biopsies Utilizing the Advanced Breast Biopsy Instrumentation System.” Am J Surg. 1997; 174: 297-302.
(7) Ferzli, George S., et al. “Advanced Breast Biopsy Instrumentation: A Critique.” J Am Coll Surg 1997;185a: 145-151.

Other biopsy devices have been referred to as the Mammotome and the Minimally Invasive Breast Biopsy (MIBB). These devices carry out a vacuum-assisted core biopsy wherein fragments of suspect tissue are removed with an 11 to 14-gauge needle. While being less invasive, the Mammotome and MIBB yields only a fragmentary specimen for pathological study. These devices therefore are consistent with other breast biopsy devices in that the degree of invasiveness of the procedure necessarily is counterbalanced against the need for obtaining a tissue sample whose size and margins are commensurate with pathology requirements for diagnosis and treatment.

Another excisional biopsy device is described in U.S. Pat. No. 6,022,362, and includes a tubular member having a window near a distal tip thereof; a cutting tool, a distal end of the cutting tool being attached near the distal tip of the tubular member, at least a distal portion of the cutting tool being configured to selectively bow out of the window and to retract within the window; and a tissue collection device externally attached at least to the tubular member, the tissue collection device collecting tissue excised by the cutting tool as the biopsy device is rotated and the cutting tool is bowed. An excisional biopsy method for soft tissue includes the steps of inserting a generally tubular member into the tissue, the tubular member including a cutting tool adapted to selectively bow away from the tubular member and an external tissue collection device near a distal tip of the tubular member; rotating the tubular member; selectively varying a degree of bowing of the cutting tool; collecting tissue severed by the cutting tool in the tissue collection device; and retracting the tubular member from the soft tissue. The tubular member may include an imaging transducer and the method may include the step of displaying information received from the transducer on a display device and the step of varying the degree of bowing of the cutting tool based upon the displayed information from the imaging transducer. Alternatively, the imaging transducer may be disposed within a removable transducer core adapted to fit within the tubular member.

Yet another minimally invasive approach to accessing breast lesions wherein the lesion is partially removed or removed in its entirety for diagnostic as well as therapeutic purposes has been described in U.S. Pat. No. 6,277,083 by Eggers, et al., entitled “Minimally Invasive Intact Recovery of Tissue”, issued Aug. 21, 2001. The instrument described includes a tubular delivery cannula of minimum outer diameter, the tip of which is positioned in confronting adjacency with a tissue volume to be removed. Following such positioning, the electrosurgically excited leading edge of an electrically conducting cable supported at the distal ends of leaf members is extended forwardly from the instrument tip to enlarge while the electrosurgically cutting and surrounding or encapsulating a tissue volume, severing it from adjacent tissue. Following such electrosurgical cutting, the instrument and the captured tissue volume are removed through an incision of somewhat limited extent. The electrosurgical cutting requires current flow from the cable to and through the surrounding tissue to maintain an electrical arc between the cables that is achieved by maintaining the cable at an elevated peak-to-peak voltage of at least 1000 volts relative to tissue. In order to enable current flow through the tissue, the elevated voltage must be applied at an alternative current frequency of at least 300 kHz in order to enable current flow from the cable through the surrounding tissue to a return electrode usually attached to the skin surface of the patient in the form of a pad having a surface area of at least 20 square inches. In this prior art, the voltage is maintained at a predetermined constant level (e.g., 1000 volts peak-to-peak), which the current flow from the cable and into the surrounding tissue is variable depending on the electrical resistivity of the surrounding tissue. The current flow from the cable into the surrounding tissue is higher for the case of denser, more fibrous tissue while the current flow from the cable into the surrounding tissue is lower for the case of fatty tissue. Maintaining an adequate current flow into the surrounding tissue to sustain an arc and associated cutting effect requires a sufficiently high voltage to overcome the electrical impedance of adjacent tissue having a very high fat content (e.g., fatty breast tissue).

An improved design for the instrument described in U.S. Pat. No. 6,277,083 is described in U.S. Pat. No. 6,471,659 by Eggers, et al., entitled “Minimally Invasive Intact Recovery of Tissue”, issued Oct. 29, 2002. This instrumentation includes a tubular delivery cannula of minimum outer diameter, the tip of which is positioned in confronting adjacency with the target tissue volume to be removed. Such positioning is facilitated through the utilization of a forwardly disposed precursor electrosurgical electrode assembly. Located within the interior channel of this delivery cannula are five relatively elongate thin leaf members mutually interconnected at their base to define a pentagonal cross-sectional configuration. Each of the five leaf members terminates forwardly at a tip with a transversely bent eyelet structure. Slideably extending through each eyelet is a separate electrically conductive electrosurgical cutting and pursing cable, which extends to an attachment with the next adjacent leaf tip. The five separate cables extend rearwardly through five small guide tubes attached to each of the five separate leafs for connection with the slideable cable terminator component of a drive assembly. The drive assembly is driven forwardly by an electric motor through a translation assembly. By adjusting the location of a stop component, which engages the cable terminator component, the size of a captured specimen may be varied. For example, the device can be configured to recover tissue specimens of between 10 mm and 15 mm effective maximum diametric extent. As the cable terminator component is pulled by the cable assembly into abutting engagement with the stop component, the cables are tensioned to draw the leaf eyelet structures together in a pursing action.

Cabling involved with the instrument specified in U.S. Pat. No. 6,471,659 must be quite diminutive in size while retaining adequate tensile strength in the temperature environment of an electrosurgical cutting arc. The electrosurgical arc temperature has been reported to be at least 1000° C. Heretofore, cable having a nominal diameter of 0.006 inch has been employed. While this electrosurgical cutting arc is present, the cables further must sustain not only stresses associated with the forward movement of the leaf members but also those loads imposed by the pursing activity during which the eyelets are drawn together to complete encapsulation of the tissue sample. For discussion of temperatures associated with electrosurgical arcs, see:
(8) Brown, B. H., et. al., “Medical Physics and Biomedical Engineering”. Taylor & Francis Group, New York 1999: 238-239
(9) Woloszko, J., et. al., “Coblation in Otolaryngology”. Proceedings of the SPIE 2003; 4949:341-352

The prior art methods other than excision using surgically sharp cutting blades (e.g., open surgery excision for biopsy or lumpectomy, ABBI method) utilize a cutting method known as electrosurgical tissue cutting (or often incorrectly referred to as “electrocautery” tissue cutting). For discussion of tissue cutting with electrosurgical arcs, see:
(10) Pearce, J. A., “Electrosurgery”. John Wiley & Sons, New York 1986 (ISBN 0-471-85435-2); 67

In this modality of tissue cutting also known as monopolar electrosurgical cutting of tissue, a large electrical potential difference is imposed between the cutting member or active monopolar electrode (e.g., a flexible wire or multi-wire cable) and a passive or return electrode placed on the surface of the patient's body, typically a voltage difference in the range from 500 to 2000 volts peak-to-peak at a frequency ranging from 250 KHz to 5 MHz. This large potential difference allows the formation of electrical arcs between the cutting member and the adjacent tissue. At the point of impingement of the cutting arcs with the surrounding tissue, highly concentrated Joulean heating within the electrically conductive tissue occurs due to the very high current flux in the tissue at the point of impingement of the arcs with the tissue. This highly localized heating at the point of arc impingement causes the cellular fluid within the tissue cells to vaporize thereby fracturing the cellular walls and effecting the separation of the tissue along the advancing pathway of the electrosurgically induced electrical arcs.

In addition to the very high temperatures associated with the formation of electrical arcs during the process of electrosurgical cutting, which can lead to the failure of thin cutting wire or cable members, methods and apparatus which utilize electrosurgical cutting can also result in aberrant current flow in the tissue beyond the point of impingement by the arcs. As a consequence, electrical currents flowing from the active electrode (e.g., the flexible cutting wire or cable) to the passive electrode (e.g., the return electrode) placed on the surface of the patient's skin can induce unintended thermal damage to both the surrounding, un-excised tissue as well as the circumscribed tissue being excised for the purpose of diagnostic pathological evaluation. Furthermore, the electrical currents flowing from the active or monopolar electrode (e.g., the flexible cutting wire or cable) to the passive electrode (e.g., the return electrode) placed on the surface of the patient's skin can cause unintended stimulation of nerve tissue beyond the zone of the applied localized anesthesia (e.g., by interstitial injection of agents such as Lidocaine) resulting in significant discomfort to the patient during the electrosurgical cutting procedure. For discussion of the potential for iatrogenic injury to the patient and damage to excised pathology specimens associated with the use with electrosurgery, see:
(11) Miller E., et. al., “Scalpel versus Electrocautery in Modified Radical Mastectomy”. American Journal of Surgery 1988; 54:284-286 Mandrekas A. D., et. al., “Fat Necrosis Following Breast Reduction” Br. J. Plastic Surgery 1994; 47:560-562
(12) Rosen, P. P., “Breast Biopsy and Electrocautery” (Letter to the Editor) Annals of Surgery 1986; 204(5):612-613

Also, in addition to the disadvantages described above related to the use of electrosurgery for the cutting and excision of breast tissue as described in U.S. Pat. Nos. 6,277,083 and 6,471,659, another limitation is related to the significant difference in the electrical resistivity of the tissue being cut. As described above, the process of electrosurgical cutting requires the flow of electrical current from the point of impingement of the electrosurgical arc to the return electrode placed on the surface of the patient's skin. However, for the case of excision of breast tissue as described in U.S. Pat. Nos. 6,277,083 and 6,471,659, the electrical resistance of the breast tissue can differ by a factor of almost ten-fold as a result of the electrical properties inherent in regions of highly adipose breast tissue in contrast to very dense breast tissue. As a consequence, electrosurgical cutting may be inadequate in some patients with highly adipose breast tissue. For a discussion of the electrical resistivity or related properties of human tissue, see:
(14) Faes, T. J., et. al., “The Electrical Resistivity of Human Tissues (100 Hz-10 MHZ): A Meta-Analysis of Review Studies” Physiological Measurements 1999; 20(4):R1-R10
(15) Geddes, L. A., et. al., “The Specific Resistance of Biological Matter-A Compendium of Data for the Biomedical Engineer and Physiologist” Medical & Biological Engineering 1967; 5:271-293

Improvements in apparatus for minimally invasive diagnostic and therapeutic excision of tissue are described in U.S. Pat. No. 11,737,808. This apparatus incorporates the use two rigid tubes to provide a conduit for first and second tensioning portions of a cutting and pursing cable. The distal end of each rigid tube provides for electrical contact with a flexible cable that is sliding over the end of each rigid tube. The rigid tubes allow only the adjacent flexible leaf members to deploy radially outward while the rigid tubes deploy only along the longitudinal axis as seen in FIGS. 18A and 18B of U.S. Pat. No. 11,737,808 thereby disadvantageously requiring the excision of a significantly larger portion of healthy tissue in order to obtain the targeted tissue volume as seen in FIG. 18C. Consequently, the use of two rigid electrically and thermally conductive tubes as the electrodes and conduits for the tensioning portions of the cutting and pursing cable prevents the incision and capturing the preferred nearly spherical tissue specimen for diagnostic and therapeutic purposes while sparing healthy tissue.

Another limitation of the apparatus for minimally invasive diagnostic and therapeutic excision of tissue described in U.S. Pat. No. 11,737,808 is the problem of intermittent loss of adequate electrical contact at the sliding interface between the cutting and pursing cable and one or both distal ends of the rigid tubes. The intermittent loss of adequate electrical contact at the sliding interface between the cutting and pursing cable and one or both distal ends of the rigid tubes results in a corresponding increase in the electrical contact resistance at the sliding contact between the cutting and pursing cable and the source of constant current at the one or both distal ends of the rigid tubes. An increase in the electrical contact resistance at the sliding interface between the cutting and pursing cable and one or both distal ends of the rigid tubes has been observed to be sufficiently large during the initial phase of deployment to cause overheating and failure of the cutting and pursing cable. During the initial phase of deployment of the rigid tubes and the adjacent leaf members, the tension applied to the cutting and pursing cable must be maintained at a minimum level in order to achieve the required slope or ramp of deployment of the leaf members and avoid premature pursing down of the distal ends of the leaf members. Consequently, during the initial phase of deployment to attain the maximum opening of the leaf members, the low level of allowed cable tension increases the likelihood that the electrical contact resistance at the sliding interface between the cutting and pursing cable and one or both distal ends of the rigid tubes may occasionally and momentarily become sufficiently large to induce excessive resistance heating at the sliding contact interface leading to overheating and melting of the cutting and pursing cable.

An objective of the present disclosure is to enable minimally invasive excision of a defined volume of tissue while overcoming or greatly limiting the disadvantageous effects described above which are associated with electrosurgical tissue cutting and excision.

In addition, an objective of the present disclosure is to enable minimally invasive excision of a nearly spherical volume of tissue and minimizing the unnecessary removal of any adjacent healthy tissue. A further objective of the present disclosure is to detect one or more occurrences of a significant increase in the electrical contact resistance at the sliding interface between the source of constant current and the cutting and pursing cable during its the deployment. Upon detection of a significant increase in the electrical contact resistance at the sliding interface between the source of constant current and the cutting and pursing cable, it is a further objective of the present disclosure to interrupt the application of constant current for a brief period until an acceptable level of electrical contact at one or both sliding interfaces is re-established and the application of the constant current can be resumed, thereby avoiding overheating and failure of the cutting and pursing cable at the locus of its sliding contact with the source of constant current.

### BRIEF SUMMARY OF THE INVENTION

The present disclosure is addressed to apparatus, system and method for retrieving a tissue volume having an intact form utilizing minimally invasive surgical instrumentation. This instrumentation includes a tubular delivery cannula of minimum outer diameter, the tip or distal end of which is positioned in confronting adjacency with the target tumor or tissue volume to be excised. The tubular delivery cannula is disposed at the distal end of a single-use support housing that is inserted into the receiving cavity of a reusable housing or handpiece assembly. Such positioning of the delivery cannula is facilitated through the utilization of a forwardly disposed cutting blade assembly supporting a surgically sharp blade.

By way of example and without limitation, located within the interior channel of this delivery cannula are two or more, preferably five, flexible leaf members and one multi-lumen flexible polymeric extrusion member. As used herein, the term polymeric, as it refers to the multi-lumen flexible extrusion, includes flexible synthetic polymers that exhibit, by way of example, the following characteristics: [a] biocompatibility with respect to direct contact with human tissue, blood and other bodily fluids, [b] sterilizable by way of example, using radiation (e.g., gamma radiation) or ethylene oxide [c] electricaly insulative having a volume electrical resistivity of preferably greater than 1×10¹² ohm-cm, [d] flexibility of the extruded member and [e] extrudable in configurations having multiple lumens, thickness as small as 0.020 inch and widths as small as 0.090 inch. Examples of polymeric materials suitable for the multi-lumen flexible polymeric extrusion disclosed herein include polyamides (e.g., Nylon 6, 11 and 12), polyethylene, fluorinated ethylene propylene and polytetrafluoroethylene.

Each leaf member is preferably formed by photochemically etching a thin metallic sheet using a known biocompatible metal having a high modulus of elasticity and yield strength such as full-hard austenitic stainless steel. The leaf member may incorporate a multiplicity of slots or perforations in order to provide the required stiffness during deployment to the maximum opening position (i.e., basket deployment phase) of the leaf members while limiting the stiffness of the leaf members during the purse down phase of the tissue capture procedure.

The multi-lumen flexible polymeric extrusion members is formed using an electrically insulative, biocompatible extrudable polymer (e.g., nylon or polytetrafluoroethylene). The multi-lumen flexible polymeric extrusion member incorporates a multiplicity of conduits, preferably four conduits. The leaf members and the multi-lumen flexible polymeric extrusion members are mutually supported and secured at their proximal ends on the perimeter surface of a leaf and multi-lumen flexible polymeric extrusion support member to define a polygonal cross-sectional configuration.

By way of example, each of the five leaf members terminate forwardly with an eyelet-containing tip. In addition, each leaf member is covered by a thin, flexible biocompatible electrically insulative coating (e.g., Parylene HT) capable of withstanding temperatures of up to at least 300° C., preferably up to 400° C. in order to prevent unwanted electrical current flow between the leaf members, between the leaf members and surrounding structures within the delivery cannula, and also to prevent unwanted electrical current flow between individual leaf members and the surrounding tissue during the conduction of a substantially constant level of electrical current through the cutting and pursing cable segments that extend between and supported within the eyelets at the distal end of each leaf member and the multi-lumen flexible polymeric extrusion member. Since the multi-lumen flexible polymeric extrusion member is formed using an electrically insulative polymer (e.g., nylon or polytetrafluoroethylene), no electrically insulative coating is required for the exterior of the multi-lumen flexible polymeric extrusion member.

The apparatus for retrieving a tissue volume of given peripheral extent includes a cutting and pursing cable that is flexible, electrically conductive and is of fixed length, the length of the flexible cutting and pursing cable having two functional regions. Throughout the present disclosure, the term “cutting and pursing cable” refers to a cable or single wire that is flexible, having sufficient flexibility to enable passage of the cutting and pursing cable (or wire) through bend radii as small as 0.030″.

A first functional region is the tensionable portion of the cutting and pursing cable and is that portion of the cutting and pursing cable in which no electrical current is conducted and that is proximal to each of first and second electrically and thermally conductive eyelets located at the distal end of the multi-lumen flexible polymeric extrusion assembly. A second functional region is the resistively heated portion of the cutting and pursing cable and is that portion of the cutting and pursing cable in which electrical current is conducted and that is distal to first and second electrically and thermally conductive eyelets located at the distal end of the multi-lumen flexible polymeric extrusion assembly. Each first and second electrically and thermally conductive eyelet is disposed at the distal end of first and second electrically and thermally conductive wires, respectively, and function as first and second electrodes, respectively, for conducting electrical current from first and second electrically and thermally conductive wires to the resistively heated portion of the cutting and pursing cable at the locus of sliding electrical contact between the cutting and pursing cable and each first and second electrically and thermally conductive eyelet as the cutting and pursing cable is advanced or retracted through the each first and second electrically and thermally conductive eyelets during the retrieval of a tissue volume.

The functional transition between the tensionable portion of cutting and pursing cable and the resistively heated portion of cutting and pursing cable is the location of sliding electrical contact between the cutting and pursing cable and each of the first and second electrically and thermally conductive eyelets. During the excision of a targeted volume of tissue, the component lengths of the first and second tensionable portions of the cutting and pursing cable decrease while the component length of the resistively heated portion of the cutting and pursing cable increase as the ensemble of leaf members and the extrusion member that form the tissue capture basket extend forwardly to a maximum peripheral extent of the tissue capture assembly. Upon the tissue capture basket having extended forwardly to a maximum peripheral extent, the component lengths of the first and second tensionable portions of the cutting and pursing cable increase while the component lengths of the resistively heated portion of the cutting and pursing cable decrease as the ensemble of leaf members and the extrusion member that form the tissue capture basket extend forwardly while an increased level of tension is applied to the cutting and pursing cable to effect the pursing down of the tissue capture assembly.

Throughout a first period when the tissue capture basket is extended forwardly to a maximum peripheral extent (i.e., capture basket opening phase) and throughout a second period when the tissue capture basket is extended forwardly and pursed down to a minimum peripheral extent, sufficient contact pressure is maintained between the cutting and pursing cable and each of the first and second electrically and thermally conductive eyelets to maintain good electrical contact at the sliding interface between the cutting and pursing cable and each of the first and second electrically and thermally conductive eyelets. Sufficient contact pressure is maintained between the cutting and pursing cable and each of the first and second electrically and thermally conductive eyelets by applying a minimum level of tension to the tensioning portions of the cutting and pursing cable, preferably an applied tension level of not less than 7 grams to each of the first and second tensioning portions of the cutting and pursing cable. The application of at least a minimum level of tension to the tensioning portions of the cutting and pursing cable allows good electrical contact to be maintained at the sliding interface between the cutting and pursing cable and each of the first and second electrically and thermally conductive eyelets disposed at the distal end of the multi-lumen flexible polymeric extrusion assembly as the multi-lumen flexible polymeric extrusion assembly is advanced through the arcuate path that defines the hemispherical shape of the proximal end of the tissue capture basket.

Extending between the multi-lumen flexible polymeric extrusion member and each adjacent leaf member is a small-diameter, resistively heated portion of a cutting and pursing cable comprising multiple wires (e.g., 7 to 19 wires) or formed from a single small-diameter wire. Hereinafter, the term “cutting and pursing cable” will commonly be used but does not exclude the possibility of the alternative use of an electrically conductive single wire in place of a cable comprising a multiplicity of wires. By way of example, the composition of the single wire may be austenitic stainless steel, nickel alloy, cobalt/nickel alloy, titanium or titanium alloy.

By way of example, the multi-lumen flexible polymeric extrusion assembly comprises an electrically insulative flexible polymeric extrusion having four lumens. A first electrically and thermally conductive lead wire (e.g., high purity silver wire) extends from a current source through a first lumen and a second electrically and thermally conductive lead wire (e.g., high purity silver wire) extends from a current source through a second lumen. A first tensionable portion of the cutting and pursing cable (e.g., a cable comprising seven wires of a cobalt/chrome/tungsten/nickel alloy such as L605) extends through a third lumen and a second tensionable portion of a cutting and pursing cable (e.g., a cable comprising seven wires of a cobalt/chrome/tungsten/nickel alloy such as L605) extends through a fourth lumen. That portion of the distal end of the first electrically and thermally conductive lead wire that extends beyond the distal end of the multi-lumen flexible polymeric extrusion member forms a first electrically and thermally conductive eyelet through which the first tensionable portion of cutting and pursing cable extends, the first tensionable portion of cutting and pursing cable making sliding electrical contact with first electrically and thermally conductive eyelet located at the distal end of the first electrically and thermally conductive lead wire. Likewise, that portion of the distal end of the second electrically and thermally conductive lead wire that extends beyond the distal end of the multi-lumen flexible polymeric extrusion member forms a second electrically and thermally conductive eyelet through which a second tensionable portion of cutting and pursing cable extends, the second tensionable portion of cutting and pursing cable making sliding electrical contact with second electrically and thermally conductive eyelet located at the distal end of the second electrically and thermally conductive lead wire. Those portions of the first and second electrically and thermally conductive lead wires located at the proximal end of the multi-lumen flexible polymeric extrusion member are connected to first and second single-use housing lead wires, respectively.

By way of example, a tissue cutting and capture assembly forms a circumferential sequence of cutting and pursing cable support members. The tissue cutting and capture assembly comprise, in sequence, the multi-lumen flexible polymeric extrusion, a first leaf member, a second leaf member, a third leaf member, a fourth leaf member and a fifth leaf member.

The first tensionable portion the cutting and pursing cable extends from the third lumen within the multi-lumen flexible polymeric extrusion and passes through the first eyelet at the distal end of the first electrically and thermally conductive lead wire that extends beyond the distal end of the multi-lumen flexible polymeric extrusion. That portion of the cutting and pursing cable that extends distally from the first eyelet at the distal end of the first electrically and thermally conductive lead wire functions as a resistively heated portion of the cutting and pursing cable and is that portion of the cutting and pursing cable in which electrical current is conducted. The portion of the cutting and pursing cable through which electrical current is conducted extends distally from the first eyelet at the distal end of the first electrically and thermally conductive lead wire continues, in sequence, through the eyelet at the distal end of the first leaf member, through the eyelet at the distal end of the second leaf member, through the eyelet at the distal end of the third leaf member, through the eyelet at the distal end of the fourth leaf member, through the eyelet at the distal end of the fifth leaf member and continues to the second eyelet at the distal end of the second electrically and thermally conductive lead wire that extends beyond the distal end of the multi-lumen flexible polymeric extrusion. In addition to electrical current flow in the cutting and pursing cable that is distal to the first and second eyelets, the total path of electrical current flow in the cutting and pursing cable also includes [a] the electrical current flow across the interface between the first eyelet at the distal end of the first electrically and thermally conductive lead wire and the portion of the cutting and pursing cable that extends distally to the first eyelet and [b] the electrical current flow across the interface between the second eyelet at the distal end of the second electrically and thermally conductive lead wire and the portion of the cutting and pursing cable that extends distally to the second eyelet. This total path of electrical current flow in the cutting and pursing cable is hereinafter referred to as the resistively heated cutting and pursing cable circuit. The second tensionable portion of the cutting and pursing cable is that portion of the cutting and pursing cable that extends through and is proximal to the second eyelet at the distal end of the second electrically and thermally conductive lead. The second tensionable portion of the cutting and pursing cable continues rearwardly through the fourth lumen in the multi-lumen flexible polymeric extrusion.

The first and second tensioning portions of the cutting and pursing cable slideably extend through a third and fourth lumen, respectively, within the multi-lumen flexible polymeric extrusion and continue to a first and second electrically and thermally conductive eyelet, respectively, disposed at the distal ends of the first and second electrically and thermally conductive lead wires, respectively, that extend through the first and second lumens, respectively, of the multi-lumen flexible polymeric extrusion as described above. Importantly, electrical current only flows in that portion of the cutting and pursing cable that extends distally to the first and second lumens of the multi-lumen flexible polymeric extrusion and beyond the point of sliding electrical contact with the first and second electrically and thermally conductive eyelets. In this manner, electrical current only flows in that portion of the cutting and pursing cable that is in contact with the tissue being incised and does not flow proximally to the first and second electrically and thermally conductive eyelets (i.e., in the first and second tensioning portions of the cutting and pursing cable that are located, respectively, within the third and fourth lumen of a multi-lumen flexible polymeric extrusion). In this manner, electrical current only flows in those portions of the cutting and pursing cable that are in thermal contact with tissue wherein the resistive heating generated within the cutting and pursing cable dissipates. The amount of heat generated within the cutting and pursing cable and transferred to the tissue in contact with the resistively heated cutting and pursing cable is sufficient to thermally sever the tissue that is in contact with the resistively heated cutting and pursing cable.

Preventing the flow of electrical current in those portions of the cutting and pursing cables that are in within the lumens of the multi-lumen flexible polymeric extrusions thereby prevents overheating the cutting and pursing cables as well as the surrounding multi-lumen flexible polymeric extrusion due to the limited ability to dissipate resistive heating within any portions of the cutting and pursing cable that is not in direct contact with the tissue being incised.

By way of example, a hexagonal shaped pattern of the resistively heated cutting and pursing cable segments is formed for the case of one multi-lumen flexible polymeric extrusion in combination with five leaf members. The hexagonal shaped pattern of the resistively heated cutting and pursing cable segments thereby form a substantially complete circumscribing cutting and pursing cable path as the segments are advanced through the tissue being incised (i.e., severed).

Each resistively heated cutting and pursing cable segment extends between either the first and second electrically and thermally conductive eyelets and the eyelets disposed at the distal ends of the nearest first and fifth adjacent leaf member, respectively, or between the eyelets disposed at the distal ends of the first and second leaf member, the eyelets disposed at the distal ends of the second and third leaf member, the eyelets disposed at the distal ends of the third and fourth leaf member and the eyelets disposed at the distal ends of the fourth and fifth leaf member.

The proximal ends of the two tensionable cutting and pursing cable portions continue rearwardly through third and fourth lumens within the multi-lumen flexible polymeric extrusions where their proximal ends are securely attached to an electrically insulative cable mounting hub.

The first electrically and thermally conductive lead wire (e.g., high purity silver wire) that extends from a current source through a first lumen within the multi-lumen flexible polymeric extrusion is electrically isolated from a second electrically and thermally conductive lead wire (e.g., high purity silver wire) that extends from a current source through a second lumen within the multi-lumen flexible polymeric extrusion. The only path for electrical current flow from the first electrically and thermally conductive lead wire to the second electrically and thermally conductive lead is through that portion of the cutting and pursing cable that is distal to the first and second eyelets at the distal ends of the first and second electrically and thermally conductive lead wires, respectively.

The first electrically and thermally conductive lead wire and the second electrically and thermally conductive lead wire serve four functions. A first function for the first and second electrically and thermally conductive lead wires is to support an electrically and thermally conductive eyelet formed at the distal end of each lead wire. Each electrically and thermally conductive eyelet provides a low-friction pathway for the passage of the cutting and pursing cable as it extends from the orifice of a third or fourth lumen within the multi-lumen flexible polymeric extrusion member to the eyelet of an adjacent leaf member.

A second function for each first and second electrically and thermally conductive lead wires is to electrically conduct a predetermined level of constant current from the electrically and thermally conductive eyelet at the distal end of each electrically and thermally conductive lead wire through a low electrical resistance sliding contact between the electrically and thermally conductive eyelet and the cutting and pursing cable as it emerges from each of the electrically and thermally conductive eyelets. Hereinafter, the term “constant current” refers to a constant electrical current. The application of a predetermined level of constant current to the resistively heated cutting and pursing cable causes the temperature of the resistively heated cutting and pursing cable to increase to a predetermined level sufficient for the controlled thermal incision of tissue by the resistively heated portion of the cutting and pursing cable without overheating and melting or fracturing the resistively heated portion of the cutting and pursing cable.

Accordingly, the predetermined level of constant current is supplied by a current source located within the handpiece assembly and operating at a frequency of at least 25 kHz, preferably at least 100 KHz. The constant current preferably operates at an elevated frequency in order to prevent unwanted electrical stimulation of tissue in contact with the resistively heated portion of the cutting and pursing cable. The constant current is delivered through a first pair of contacts on the interior of the reusable handpiece assembly and an aligned second pair of contacts on the exterior of the single-use support housing and finally through lead wires within the single-use support housing that extend from the second pair of contacts on the exterior of the single-use support housing to the proximal ends of the first and second electrically and thermally conductive lead wires. In this manner, the supply of a constant level of electric current flows through low electrical resistance leads, low electrical resistance contacts and low electrical resistance paths along the length of the first and second electrically and thermally conductive lead wires located within lumens in the multi-lumen flexible polymeric extrusion assembly. The electrical current only commences its flow through the relatively higher resistance cutting and pursing cable as it exits the first or second electrically and thermally conductive eyelets at the distal ends of each of the first and second electrically and thermally conductive lead wires, respectively, and then into to the cutting and pursing cable.

Throughout the present disclosure, the term “electrical resistance” and “electrical impedance” are used interchangeably. The more widely used designation for the “electrical resistance” of electrical circuits wherein an alternating current is flowing is “electrical impedance”. The term electrical impedance in circuits wherein alternating current is flowing includes the effects of the induction of voltages in conductors by the magnetic fields (inductance), and the electrostatic storage of charge induced by voltages between conductors (capacitance). The impedance component caused by these two effects is collectively referred to as reactance and is additive to the electrical resistance of the circuit associated with direct current or “DC” flow.

A third function for each first and second electrically and thermally conductive lead wire contained within lumens extending the length of the multi-lumen flexible polymeric extrusion assembly is to conduct heat from the first and second electrically and thermally conductive eyelets at the distal end of the first and second electrically and thermally conductive lead wires to the proximal portions of the first and second electrically and thermally conductive lead wires thereby minimizing the temperature rise of the first and second electrically and thermally conductive lead wires at the distal end of the multi-lumen flexible polymeric extrusion.

A fourth function for each first and second electrically and thermally conductive lead wire contained within lumens extending the length of the multi-lumen flexible polymeric extrusion assembly is to increase the column strength of a multi-lumen flexible polymeric extrusion formed using elastomeric materials having a low modulus of elasticity such as, by way of example, nylon or fluoropolymer materials. The increased column strength enables the multi-lumen flexible polymeric extrusion formed using elastomeric materials having a low modulus of elasticity to maintain its longitudinal configuration within the delivery cannula during the deployment of the multi-lumen flexible polymeric extrusion assembly.

A fifth function for each first and second electrically and thermally conductive lead wire contained within lumens extending the length of the multi-lumen flexible polymeric extrusion assembly is to guide the movement of the cutting and pursing cable.

The individual lumens within the multi-lumen flexible polymeric extrusion member are electrically isolated from each other since the flexible polymeric extrusion member is formed of an electrically insulative material such as nylon or polytetrafluorethylene. The electrical isolation of the first and second lumens through which the first and second electrically and thermally conductive leads extend prevents electrical current flow within the extrusion between the first and second electrically and thermally conductive leads even though the constant current applied to the first and second electrically and thermally conductive leads results in a voltage differential between the first and second electrically and thermally conductive leads of opposite polarity ranging from several volts to several tens of volts.

No significant electrical resistive heating occurs until the applied constant current flow begins within those portions of the relatively high electrical resistance cutting and pursing cable that extend into and that are in direct contact with the surrounding tissue that is positioned beyond the electrically and thermally conductive eyelets at the distal ends of the first and second electrically and thermally conductive lead wires. Importantly, the distal end of the delivery cannula of the tissue incision and retrieval assembly that abuts the targeted tissue is configured so that those portions of the cutting and pursing cable that extend beyond the electrically and thermally conductive eyelets at the distal ends of the first and second electrically and thermally conductive lead wires are always in direct thermal contact with soft tissue and/or fluids (e.g., blood). Additionally, the distal end of the tissue incision and retrieval assembly that abuts the targeted tissue may initially be advanced a short distance without any applied constant current within the resistively heated portion of the cutting and pursing cable to assure good thermal contact between the cutting and pursing cable with the targeted tissue prior to the commencement of resistive heating within the portion of the cutting and pursing cable that is distal to the first and second electrically and thermally conductive eyelets at the distal ends of the first and second electrically and thermally conductive lead wires.

According to the teachings of this disclosure, a tissue cutting and capture assembly may comprise two or more leaf members, one multi-lumen flexible polymeric extrusion assembly and a resistively heatable cutting and pursing cable that is resistively heatable along those portions of the cutting and pursing cable that are in direct contact with tissue and distal to the first and second electrically and thermally conductive eyelets at the distal ends of the first and second electrically and thermally conductive lead wires, respectively, The two or more leaf members and the multi-lumen flexible polymeric extrusion assembly are secured at their respective proximal ends to a leaf member and extrusion assembly support member.

The leaf member and extrusion assembly support member is driven forwardly by a motor-actuated drive tube drive member translation assembly that is abuttingly engaged against the leaf member and extrusion assembly support member to actuate the deployment of the tissue cutting and capture assembly. The tissue cutting action is enabled by the passage of electrical current only through those segments or sections of the resistively heated portion of cutting and pursing cable that are in direct contact with tissue or fluids within the body. Hereinafter, references to contact with tissue may include contact with tissue and other fluids within the body. The electrical current passing through only those portions of the resistively heated portion of cutting and pursing cable in direct contact with tissue is of sufficient current flux to induce resistive heating of the cutting and pursing cable to achieve an elevated temperature sufficient to establish a thermally induced cutting effect at the leading edge of the resistively heated portion of cutting and pursing cable. By way of example, the current flux and associated resistance heating is sufficient to maintain the temperature of the resistively heated portion of cutting and pursing cable at a temperature of at least 300° C., preferably at least 400° C., at a predetermined tissue cutting rate, R_{cut} and types of soft tissue media including, but not limited to, muscle tissue, adipose tissue, tendons, lymphatic tissue as well as transecting blood vessels and exposure to blood and other liquids within the body.

An essential attribute of the apparatus of the present disclosure is the confinement of the path of electrical conduction of constant current required to achieve tissue cutting to only those portions of the expanding and contracting resistively heated segments of the cutting and pursing cable that are distal to the electrically and thermally conductive eyelets at the distal ends of the first and second electrically and thermally conductive lead wires located at the distal end of the multi-lumen flexible polymeric extrusion assembly and that are in direct contact with tissue. The confinement of the path of electrical conduction of constant current to only those portions of the expanding and contracting resistively heated segments of the cutting and pursing cable that are in direct contact with tissue avoids overheating those portions of the tensionable cutting and pursing cable that are located proximal to the electrically and thermally conductive eyelets at the distal ends of the first and second electrically and thermally conductive lead wires and within the third and fourth lumens of the multi-lumen flexible polymeric extrusion assembly and not in contact with tissue. As a consequence, the rate of heat dissipation from the cutting and pursing cable is negligible in those proximal portions of the cutting a pursing cable that are not distal to the eyelets and that are not in contact with tissue.

The tensionable portions of the cutting and pursing cable extending through and rearwardly within the third and fourth lumens of the multi-lumen flexible polymeric extrusion assembly enable the application of a mechanical load or tension level required for the pursing down of the tissue capture basket during the process of incising and capturing a target tissue volume. The mechanical load or tension level is uniformly applied to the proximal ends of each of the tensionable portions of the cutting and pursing cable beginning after the basket has reached the pre-selected maximum opening diameter (e.g., 25 mm diameter opening size).

The tensionable portions of the cutting and pursing cable that are not in direct contact with tissue and proximal to the tip of the tissue capture basket are not intended to support the electrical conduction of the constant current required to heat the cable above temperature threshold levels required for the thermal cutting of tissue. Importantly, electrical current flows only within the resistively heated portion of cutting and pursing cable in direct contact with tissue while no electrical current flows into or through the tissue being cut. Advantageously, the prevention of electrical current flow into or through the tissue being cut minimizes necrosis of tissue beyond the immediate surface of the tissue incision thereby assuring a pathology specimen having minimal thermal and electrical current related damage or artifact. In addition, for the case of procedures performed with only local anesthesia, the prevention of electrical current flow into or through the tissue being cut can prevent the induction of pain in the patient's nerve pathways located beyond and more distant from the path of tissue incision and beyond the localized region of induced anesthesia as with the injection of analgesic agents such as Lidocaine.

By way of example, as the two or more leaf members and the multi-lumen flexible polymeric extrusion assembly engaged with the drive assembly drive member are driven forwardly by a motor-actuated drive tube drive member translation assembly incorporating a first motor and planetary gear train assembly (hereinafter referred to as the first motor-actuated drive tube drive member translation assembly), the eyelets disposed at the distal ends of the two or more leaf members and the distal end of the multi-lumen flexible polymeric extrusion assembly are correspondingly driven forwardly. Alternatively, other means for advancement of the drive tube drive member translation assembly may be employed, including, but not limited to, pneumatic or spring-loaded advancement. The eyelets at the distal ends of the leaf members and the multi-lumen flexible polymeric extrusion assembly support the resistively heated portions of the cutting and pursing cable as the cutting and pursing cable is driven forwardly at a deployment angle mutually outwardly through a guidance assembly to an extent that the cutting leading edge of the resistively heated portion of the cutting and pursing cable reaches an effective maximum diameter extending about and circumscribing the target tissue volume to be excised and captured. By way of example, a first pivotable drive finger extending from the first motor-actuated drive tube drive member translation assembly within a handpiece assembly may drivably engage the drive assembly drive member to affect its advancement and the supported leaf members and multi-lumen flexible polymeric extrusion assembly. The pivotable aspect of first pivotable drive finger enables the single-use tissue incision and retrieval assembly to be removed from the handpiece assembly in the presence of the drive block advancement ear extending from drive assembly drive member and pivoting drive ear extending from cable mounting hub.

In a preferred embodiment, the tissue incision and retrieval system includes a practitioner-actuatable capture size selection switch on the handpiece assembly that enables the practitioner to select a diameter of a substantially spherical volume of tissue in the range, for example, from 15 mm to 30 mm in diameter increments of 5 mm. Upon the selection of the desired diameter of a substantially spherical volume of tissue by the practitioner using the capture size selection switch, the second-motor actuated cable mounting hub translation assembly advances the position of the second pivotable drive finger to a predetermined position. The predetermined position of the second pivotable drive finger controls the travel distance of the cable mounting hub along a support tube between its initial position and its position abutting the pivoting drive ear extending from the cable mounting hub. The larger the preferred diameter of a substantially spherical volume of tissue the greater the travel distance between the initial position of the cable mounting hub and its position abutting the pre-positioned second pivotable drive finger.

While the deploying leaf members and the multi-lumen flexible polymeric extrusion assembly are being advanced to attain the pre-selected diameter of the substantially spherical volume of tissue, the cable mounting hub encounters and is abuttingly engaged against the second pivotable drive finger. As the cable mounting hub advances, in conjunction with the advancement of the deploying leaf members and the multi-lumen flexible polymeric extrusion assembly, tension is applied to the first and second tensioning portions of the cutting and pursing cable. The applied tension is pre-selected to be sufficiently high to maintain low electrical contact resistance between the cutting and pursing cable as it makes sliding electrical contact with each of the electrically and thermally conductive eyelets at the distal ends of the first and second electrically and thermally conductive lead wires located at the distal end of the multi-lumen flexible polymeric extrusion assembly. Also, the applied tension is pre-selected to be sufficiently low to prevent unwanted premature pursing down of the ensemble of deploying leaf members and the multi-lumen flexible polymeric extrusion assembly prior to the attainment of the practitioner selected diameter extent of the target tissue being incised ant captured.

The practitioner-selected diameter of a substantially spherical volume of the tissue being excised corresponds to the maximum diameter reached by the distal ends of the two or more (preferably five) leaf members and the distal end of the multi-lumen flexible polymeric extrusion. The maximum diameter reached by the distal ends of the two or more (preferably five) leaf members and the distal end of the multi-lumen flexible polymeric extrusion occurs when the cable mounting hub reaches and abuts a second pivotable drive finger extending from a second motor-actuated cable mounting hub translation assembly. The second pivotable drive finger that extends from a second motor-actuated cable mounting hub translation assembly serves two functions. The first function is to determine the maximum diameter reached by the distal ends of the two or more (preferably five) leaf members and the distal end of the multi-lumen flexible polymeric extrusion. The second function is to control the rate of purse down of the cutting and capture basket. The rate of purse down of the cutting and capture basket is controlled by a pre-selected rate of rearward advancement of the second pivotable drive finger that extends from a second motor-actuated cable mounting hub translation assembly. As the second pivotable drive finger is driven rearwardly along with the abutting cable mounting hub, the first and second tensioning portions of the cutting and pursing cable that are attached to the cable mounting hub are tensioned and draw together, in a pursing action, the eyelet structures that are located at the distal ends of the two or more (preferably five) leaf members and the distal end of the multi-lumen flexible polymeric extrusion. In this pursing process, the eyelets located at the distal ends of the leaf members and multi-lumen flexible polymeric extrusion assembly are drawn mutually inwardly to define a hemispherical curvilinear profile to close the leading edge about the tissue volume as the forward movement of the leaf members and the multi-lumen flexible polymeric extrusion assembly continues. The resistively heated portion of the cutting and pursing cable, now under tension and constrained by the interior surfaces of the leaf members and multi-lumen flexible polymeric extrusion assembly contributes to the structural stability of the resultant tissue capture basket.

In a preferred method, upon reaching a predetermined electrical current level in the second motor incorporated in the second motor-actuated cable mounting hub translation assembly, a pre-selected voltage is no longer applied to the first motor-actuated drive tube drive member translation assembly, a pre-selected voltage is no longer applied to the second motor-actuated cable mounting hub translation assembly and a pre-selected level of constant current is no longer supplied to the resistively heated portion of the cutting and pursing cable. The termination of the first and second voltages applied to the first motor-actuated drive tube drive member translation assembly and the second motor-actuated cable mounting hub translation assembly as well as the termination of the constant current that is no longer supplied to the resistively heated portion of the cutting and pursing cable corresponds to the end of the excision and capture of the targeted tissue volume followed by the removal of the delivery cannula of the tissue incision and retrieval system by the practitioner along with the captured volume of tissue.

The rate of translation of the first motor-actuated drive tube drive member translation assembly that forwardly advances the leaf members and the multi-lumen flexible polymeric extrusion assembly in combination with the rate of translation of the second motor-actuated cable mounting hub translation assembly that rearwardly retracts the cable mounting hub determines the degree or extent of curvature of the noted curvilinear profile.

In a preferred embodiment, first and second electrical terminals are located on the left and right interior sides of the handpiece assembly and are in slideable electrical contact with corresponding first and second terminals located on the left and right exterior sides of the single-use support housing, respectively. These slideably communicating first and second electrode pairs provide the supply of constant current to the cutting and pursing cable. As a result of the small diameter and high electrical resistivity of the cutting and pursing cable, substantially all of the electrical resistance in the resistively heated cutting and pursing cable circuit carrying the applied constant current is confined to those portions of the cutting and pursing cable that extend from and are distal to the eyelets of the two or more leaf members and the multi-lumen flexible polymeric extrusion assembly. As the leaf members and multi-lumen flexible polymeric extrusion assembly deploy during the incision and capture of the target tissue volume, the length of the resistively heated portion of the cutting and pursing cable increases from an initial starting position until the practitioner-selected diameter of a substantially spherical volume of tissue is attained.

Once the practitioner-selected diameter of a substantially spherical volume of tissue is attained, the second pivotable drive finger induces the pursing down of the distal ends of the leaf members and multi-lumen flexible polymeric extrusion assembly, thereby causing the length of the resistively heated portion of the cutting and pursing cable to decrease to a minimum length upon the completion of the drawing together or pursing down of the distal ends of the leaf members and multi-lumen flexible polymeric extrusion assembly. As the length of the of the resistively heated portion of cutting and pursing cable increases to a maximum value during the basket opening phase followed by a decrease to a minimum value during the basket pursing down phase, the electrical resistance of the resistively heated portion of the cutting and pursing cable increases or decreases. Since a predetermined constant current is supplied to the resistively heated portion of the cutting and pursing cable during the tissue incision and capture process, the corresponding applied voltage will increase or decrease proportionately as the electrical resistance of the resistively heated portion of the cutting and pursing cable increases or decreases according to the well-known Ohm's Law. As applied to the present disclosure, Ohm's Law specifies that the voltage differential across the ends of a conductor is proportional to the product of the electrical current flow through a conductor and the electrical resistance of the conductor. Hence, although the level of electrical current supplied to cutting and pursing cable is selected to be substantially constant, the applied voltage level varies throughout the course of the tissue incision and capture process. The predetermined level of constant current is selected to achieve [a] a sufficient heat generation rate within the portions of the cutting and pursing cable that are distal to the eyelets of the multi-lumen flexible polymeric extrusion assembly and extend to the adjacent leaf member eyelets to maintain a temperature sufficient to incise tissue (e.g., a temperature of at least 300° C.) while [b] maintaining the maximum temperature of the cable, throughout the tissue incision and capture process, below the threshold of mechanical failure (i.e., breaking) of the cable under the applied tensile load or the threshold of melting the cable.

In addition to measuring the current level delivered to Motor 2 as an indication of the completion of the purse down of the leaf members and the multi-lumen flexible polymeric extrusion assembly as describe above, the voltage level applied to the resistively heated cutting cable may also be used to indicate the completion of the purse down of the leaf members and the multi-lumen flexible polymeric extrusion assembly. By way of example, the voltage level applied across the first and second constant current terminals located on the left and right interior sides of the handpiece assembly, which varies according to the length of the deployed resistively heated portions of the cutting and pursing cables as discussed above, is continuously measured throughout the tissue cutting and capture process. In addition, the predetermined constant current level supplied to the varying length of the resistively heated portions of the cutting and pursing cables that extends beyond the distal ends of the two or more leaf members and the multi-lumen flexible polymeric extrusion assembly is also continuously measured and controlled throughout the tissue cutting and capture process. Circuitry within the controller located within the handpiece assembly ratiometrically combines the measured varying voltage level and the measured constant current level to continuously derive the effective electrical resistance of the resistively heated cutting and pursing cable, R_{cable}. When the derived value of R_{cable} decrease below a predetermined minimum cable resistance value, Rₘᵢₙ, corresponding to the minimum length of deployed cutting and pursing cable that exists upon the completion of the drawing down or pursing together of the distal ends of the multi-lumen flexible polymeric extrusion assembly and all leaf members, then the process of the incision and capture of the target tissue volume is complete.

Following the completion of the incision and capture of the target tissue volume, the substantially constant current applied to the resistively heated portion of the cutting and pursing cable is discontinued. Additionally and simultaneously, the voltage applied to the first motor-actuated drive tube drive member translation assembly is discontinued and the voltage applied to the second motor-actuated cable mounting hub translation assembly is discontinued. Additionally and simultaneously, the controller within the handpiece assembly provides a visual cue on the handpiece assembly and an audible cue or tone is discontinued (i.e., the audible cue or tone being issued from the handpiece assembly throughout the incision and capture process) to indicate that the incision and capture process for circumscribing and capturing the target tissue volume is complete. At this point, the delivery cannula is removed from the patient along with a retained volume of captured tissue containing the target tissue volume.

The tissue incision and retrieval instrument of the present disclosure enjoys the capability of providing a range of maximum effective diameters during tissue incision and capture. Accordingly, the maximum effective diameter is selected by the practitioner once the single-use tissue incision and retrieval assembly is inserted into the handpiece assembly and secured into position using the rotatable locking nut positioned at the distal end of single-use support housing. Once the single-use tissue incision and retrieval assembly is inserted into the handpiece assembly and just prior to the start of a procedure, the practitioner may select the diameter of the tissue volume to be captured by manually depressing a capture size selection switch located on the handpiece assembly. By way of example, the practitioner may select a diameter for the intended incised and captured tissue volume of 15, 20, 25 or 30 mm. Upon the practitioner's selection of the intended diameter of the incised and captured tissue volume, the motor-actuated cable mounting hub translation assembly positions the second pivotable drive finger at a precise distance from the starting position of the cable mounting hub.

The relatively straightforward structuring of the delivery cannula, leaf member assembly, multi-lumen flexible polymeric extrusion assembly and drive assembly drive member permits their incorporation within a single-use support housing that is removably insertable within a manually maneuverable handpiece assembly. The first motor-actuated drive tube drive member translation assembly within the handpiece assembly may be arranged either in-line along the same longitudinal axis of the delivery cannula, leaf member assembly, multi-lumen flexible polymeric extrusion assembly and drive assembly drive member or may be arranged side-by-side relative to the longitudinal axis of the delivery cannula, leaf member assembly, multi-lumen flexible polymeric extrusion assembly and drive assembly drive member. Likewise, the second-motor actuated cable mounting hub translation assembly within the handpiece assembly may be arranged either in-line along the same longitudinal axis of the delivery cannula, leaf member assembly, multi-lumen flexible polymeric extrusion assembly and drive assembly drive member or may be arranged side-by-side relative to the longitudinal axis of the delivery cannula, leaf member assembly, multi-lumen flexible polymeric extrusion assembly and drive assembly drive member.

User control over the tissue incision and retrieval instrument of this disclosure is provided in the form of control switches located on the hand-held handpiece assembly. All practitioner usage and cueing functions are incorporated within the handpiece assembly in combination with a rechargeable battery, thereby eliminating the need for an external control assembly and associated interconnecting cable. If the tissue incision and retrieval procedure is to be performed in a sterile field (e.g., the sterile field of an operating room), then a single-use, thin, transparent and flexible sterile sheath may first be placed over the handpiece assembly, after first inserting pre-sterilized single-use handpiece assembly, to ensure that the tissue incision and retrieval instrument is effectively sterile as a result of the handpiece assembly being enclosed within a sterile sheath. By way of example, a single-use sterile sheath suitable for enveloping the handpiece assembly is available from Protek Medical Products, Inc. located in Coralville, Iowa.

In carrying out the tissue incision and retrieval procedure, the distal end of the delivery cannula is positioned in confronting adjacency with the target tissue volume to be removed. The positioning step is achieved through the utilization of a forwardly disposed, surgically sharp cutting blade assembly and guided to confronting adjacency with the target tissue volume using stereotactic, ultrasound, MRI or other guidance methods suitable for locating the target tissue volume.

The delivery cannula being thus positioned, the practitioner depresses the start tissue incision and capture switch located on the handpiece assembly to commence the incision and capture of the target tissue volume. Upon depressing the start tissue incision and capture switch, the internal control system, comprising a circuit board assembly within the handpiece assembly, enters a capture mode. At the commencement of the capture mode, an electrical current is applied exclusively and only through the resistively heated portion of the cutting and pursing cable located at the distal ends of the multi-lumen flexible polymeric extrusion assembly and leaf members. The electrical current is preferably an electrical current applied at a constant level from a current source located within the handpiece assembly. The electrical current, when applied, may be delivered continuously or in a sequence of pulses.

A predetermined level of electrical current, preferably a substantially constant current, is applied only to the resistively heated portions of the cutting and pursing cables that are in contact with tissue and the electrical current is applied in conjunction with the activation of a first motor-actuated drive tube drive member translation assembly and the activation of a second motor-actuated cable mounting hub translation assembly such that the cutting and pursing cable is advancing through tissue while constant current is being applied to the cutting and pursing cable by a constant current source. The constant current source preferably operates at a frequency of at least 25 kHz and more preferably at a frequency of 100 kHz or greater. The current source preferably delivers a substantially constant current level as an alternating current at an elevated frequency of at least 25 kHz and preferably at a frequency of 100 KHz or greater to minimize the occurrence of electrical stimulation of tissue that is in contact with the resistively heated portion of the cutting and pursing cable. However, unlike prior art devices, minimal or no electrical current flows through surrounding tissue but essentially only flows through the resistively heated portions of the cutting and pursing cables.

With the commencement of the activation of the first motor-actuated drive tube drive member translation assembly in combination with the subsequent commencement of the activation of the second motor-actuated cable mounting hub translation assembly and the delivery of a substantially constant current to the resistively heated portions of cutting and pursing cable that is in contact with tissue, the tissue cutting and capture assembly commences to be deployed from the cannula distal end assembly.

The resistively heated portion of the cutting and pursing cable can be partitioned into six segments. The six segments of the resistively heated portion of the cutting and pursing cable include, in sequence, the length of resistively heated portion of the cutting and pursing cable located between [a] the first electrically and thermally conductive eyelet located at the distal end of the first electrically and thermally conductive lead wire and the eyelet disposed at the distal end of the first (adjacent) leaf member, [b] the eyelets disposed at the distal ends of the first and second leaf members, [c] the eyelets disposed at the distal ends of the second and third leaf members, [d] the eyelets disposed at the distal ends of the third and fourth leaf members, [e] the eyelets disposed at the distal ends of the fourth and fifth leaf members and [f] the eyelet disposed at the distal end of the fifth (adjacent) leaf member and the second electrically and thermally conductive eyelet located at the distal end of the first electrically and thermally conductive lead wire.

Once the tissue incision and capture process for circumscribing and capturing the target tissue volume has been completed, the delivery cannula with captured tissue specimen is removed from the body of the patient through the same incision site used to advance the delivery cannula to the site of the target tissue. The captured tissue specimen within the enveloping tissue cutting and capture assembly, an assembly that is formed by the leaf members and the multi-lumen flexible polymeric extrusion assembly, is released from the tissue cutting and capture assembly by severing the cutting and pursing cable located at the distal end of the captured tissue specimen. By way of example, the cutting and pursing cable may be severed using a small surgical scissors such as a tenotomy scissors.

If, during the capture mode, the practitioner wishes to halt the procedure, the start tissue incision and capture switch can be depressed to cause the control assembly to enter a pause mode. In this pause mode, the current applied to the resistively heated portion of cutting and pursing cable as well as the voltage applied to the first motor-actuated drive tube drive member translation assembly and the second motor-actuated cable mounting hub translation assembly are suspended. The practitioner carries out the return to the capture mode performance by again depressing the start tissue incision and capture switch.

The control system includes one or more circuit board assemblies that incorporate a current source, first and second motor drive power sources, one or more microcomputers and a circuit to continuously measure the electrical impedance of the resistively heated cutting and pursing cable circuit. By way of example of the control system, programming within a first microcomputer enables a response to the capture actuation switch disposed on the handpiece assembly, activate the audible tones and display indicator lights and to stop the procedure when capture complete state is attained. The control system also activates a visual que to indicate when the rechargeable battery within handpiece assembly requires recharging.

By way of another example of the control system, programming within a second microcomputer within a circuit board assembly uses the continuously measured values of the electrical impedance of the resistively heated cutting and pursing cable circuit to compare the amount of increase of the continuously measured electrical impedance of the resistively heated cutting and pursing cable circuit, Δ_{CR} during a predetermined brief time interval, Δₜ with a pre-programmed maximum acceptable increase during the brief time step, Δₜ of the continuously measured electrical impedance of the resistively heated cutting and pursing cable circuit, Amax. In a preferred embodiment, the brief time interval, Δₜ is in the range from 0.001 to 0.003 second and the maximum acceptable increase of the continuously measured electrical impedance of the resistively heated cutting and pursing cable circuit, Amax is in the range from 0.3 to 0.5 ohm. If a measured increase in the electrical impedance of the resistively heated cutting and pursing cable circuit, Δ_{CR} during a predetermined brief time interval, Δₜ exceeds the maximum acceptable increase, Amax, then the increase is attributable to a sudden loss of good electrical contact between the resistively heated cutting and pursing cable and the first and/or second electrically and thermally conductive eyelets (i.e., a sudden increase in the electrical contact resistance between the resistively heated cutting and pursing cable and the first and/or second electrically and thermally conductive eyelets). Upon the detection of a sudden loss of good electrical contact between the resistively heated cutting and pursing cable and the first and/or second electrically and thermally conductive eyelets corresponding to the measured value of Δ_{CR} exceeding Δₘₐₓ during a brief time interval, Δₜ, programming within the microcomputer temporarily interrupts the supply of constant current for a predetermined time interval, Δₚₐᵤₛₑ while continuing to advance the multi-lumen flexible polymeric extrusion and leaf members to re-establish good electrical contact between the resistively heated portion of the cutting and pursing cable and the first and/or second electrically and thermally conductive eyelets by increasing the contact pressure between the resistively heated portion of the cutting and pursing cable and the first and/or second electrically and thermally conductive eyelets. In a preferred embodiment, the duration of the interruption, Δₚₐᵤₛₑ ranges from 0.10 to 0.30 second. A momentary loss of acceptable contact pressure between the resistively heated cutting and pursing cable and the first and/or second electrically and thermally conductive eyelets can result in an associated unacceptably large increase in the electrical contact resistance at the interface between the resistively heated cutting and pursing cable and the first and/or second electrically and thermally conductive eyelets thereby causing overheating of the resistively heated cutting and pursing cable and its failure through melting or a significant decrease in the load-bearing capability to withstand the tensile forces applied to the resistively heated cutting and pursing cable.

If the measured value of Δ_{CR} exceeds Δₘₐₓ during a brief time interval, Δₜ for five consecutive temporarily interruptions of the supply of constant current for the predetermined time interval, Δₚₐᵤₛₑ while continuing to advance the multi-lumen flexible polymeric extrusion and leaf members after each interruption, then programming within the second microcomputer in the circuit board assembly promptly interrupts the deployment of the tissue capture basket and begins flashing the “Capturing” indicator light, stops any further application of constant current to the resistively heated portions of the cutting and pursing cable, stops the application of voltage to the first motor and discontinues the audible tone generated by the speaker within the handpiece assembly to alert the practitioner that a malfunction has occurred.

As a result of the intentional interruption of the supply of constant current to the resistively heated cutting and pursing cable circuit upon the detection of an unacceptably large momentary increase in the measured electrical impedance within the resistively heated cutting and pursing cable circuit, the resulting high level of Joulean heating at the interface of the resistively heated cutting and pursing cable and the first and/or second electrically and thermally conductive eyelets and an associated significant increase in the localized temperature of the resistively heated cutting and pursing cable is avoided. The amount of localized Joulean heating (in watts) associated with unacceptably large electrical contact resistance at the interface between the resistively heated cutting and pursing cable and the first and/or second electrically and thermally conductive eyelets is equal to the product of the square of the applied constant current (in amps) and the unacceptably large value of the electrical contact resistance (in ohms) at the interface between the resistively heated portion of the cutting and pursing cable and the first and/or second electrically and thermally conductive eyelets.

By way of another example of the control system, programming within a second microcomputer within a circuit board assembly uses the continuously measured values of the electrical impedance of the resistively heated cutting and pursing cable circuit to continuously compute the rate of increase of electrical impedance with respect to time, Rᵣₐₜₑ during a predetermined initial period of deployment, t_{capture} of the leaf members and the multi-lumen flexible polymeric extrusion to the maximum peripheral extent of the tissue cutting and capture assembly. If the rate of increase of electrical impedance with respect to time, Rᵣₐₜₑ during a predetermined initial period of deployment, t_{capture} is below a specified threshold, then it is indicative of an insufficient diameter of the captured specimen. The computed rate of increase of electrical impedance with respect to time, Rᵣₐₜₑ during a predetermined initial period of deployment, t_{capture} is compared with a predetermined minimum rate of increase of electrical impedance with respect to time, Rᵣₐₜₑₘᵢₙ. If the computed rate of increase of electrical impedance with respect to time, Rᵣₐₜₑ during a predetermined initial period of deployment, t_{capture} is less than a predetermined minimum rate of increase of electrical impedance with respect to time, Rᵣₐₜₑₘᵢₙ, then programming within the second microcomputer in the circuit board assembly interrupts the deployment of the tissue capture basket and begins flashing the “Capturing” indicator light, stops the application of constant current to the resistively heated portions of the cutting and pursing cable, stops the application of voltage to the first motor and discontinues the audible tone generated by the speaker within the handpiece assembly to alert the practitioner that a malfunction has occurred.

A series of bench-top tests were performed to measure the temperature of a constant-current resistively heated wire (viz., solid platinum wire) using resistance thermometry while the wire is cutting and advancing through a sample of ex vivo animal tissue. The diameters of the cutting wires used in these bench-top tests were selected to be similar to the diameters of the constant-current, resistively heated portions of the cutting and pursing cables (e.g., stainless steel 316 or cobalt/tungsten/chromium/nickel alloy L605). Unlike the preferred stainless steel or cobalt-based alloys preferred for the cutting and pursing cable employed in the present invention, platinum was used in these bench-top tests since its significantly larger and well-established temperature coefficient of resistance value enables the use of resistance thermometry to determine the temperature of a heated wire during tissue cutting tests. The results of bench-top tests confirmed that the average temperature of a cutting wire or cable having overall diameters in the range from nominally 0.003 inch to 0.005 inch ranges from about 350° C. to 400° C. while cutting ex vivo animal tissue at a cutting (i.e., advancement) rate of 2.3 to 3.0 mm/second. In addition, the results of bench-top tests confirmed that the average heat flux required to cut or advance at a rate of 2.3 to 3.0 mm/second through a sample of ex vivo animal tissue ranges from 150 to 220 watts/cm². Based on the results of the cutting tests in samples of ex vivo animal tissue, the heat flux dissipated from the cutting and pursing wire employed in the tissue incision and retrieval assembly of the present disclosure is preferably at least 150 watts/cm² and more preferably at least 220 watts/cm².

Other objects of the present disclosure will be obvious and will, in part, appear hereinafter. The present disclosure, accordingly, comprises the method, system and apparatus possessing the construction, combination of elements, arrangement of parts and steps, which are exemplified in the following detailed description. For a fuller understanding of the nature and objects of the present disclosure, reference should be made to the following detailed description taken in connection with the accompanying drawings.

### BRIEF DESCRIPTION OF THE SEVERAL VIEWS OF THE DRAWINGS

For a fuller understanding of the nature and advantages of the present method and process, reference should be had to the following detailed description taken in connection with the accompanying drawings, in which:

FIG. 1 is a perspective view of the system of the invention showing a reusable, battery-powered handpiece assembly incorporating a control system, single-use tissue incision and retrieval assembly, practitioner control switches, displays and handpiece assembly battery charger;

FIG. 1A is a side view of the reusable handpiece assembly incorporating the single-use tissue incision and retrieval assembly;

FIG. 1B is a side view of the reusable handpiece assembly incorporating the single-use tissue incision and retrieval assembly enclosed within single-use sterile sheath;

FIG. 2 is a perspective view of several components of the system shown in FIG. 1 with a single-use tissue incision and retrieval assembly being shown removed from a reusable handpiece assembly;

FIG. 3 is an exploded view of the reusable handpiece assembly shown in FIGS. 1 and 2;

FIG. 4 is a sectional view of the single-use tissue incision and retrieval assembly shown in FIGS. 1 and 2;

FIG. 4A is a sectional view end of cap with O-ring providing a water-tight seal at distal end opening of handpiece assembly for use during cleaning and disinfection of handpiece assembly between surgical procedures;

FIG. 4B is a sectional view of proximal end of delivery cannula showing compliant sealing gland;

FIG. 5 is a perspective view of cable mounting hub with pivoting drive finger shown in FIG. 4;

FIG. 5A is an end view of cable mounting hub shown in FIG. 5;

FIG. 5B is a sectional side-view of cable mounting hub shown in FIG. 5;

FIG. 6 is a side view of translation nuts seen in FIG. 3;

FIG. 7 is a perspective view of first and second pivotable drive fingers seen in FIG. 3;

FIG. 7A is an end view of pivotable drive finger seen in FIG. 7;

FIG. 7B is a cross-sectional view of pivotable drive finger seen in FIG. 3;

FIG. 8 is a top view of single-use tissue incision and retrieval assembly seen in FIG. 2;

FIG. 9 is a front-end view of the single-use tissue incision and retrieval assembly seen in FIG. 2 at various stages of deployment of leaf members, multi-lumen flexible polymeric extrusion assembly and cutting and pursing cables;

FIG. 9A is a cross-sectional view of a cable construction comprising seven individual wires;

FIG. 10 is a sectional view taken through a multi-lumen flexible polymeric extrusion having round lumens as shown in FIG. 11;

FIG. 11 is a perspective view of the distal end of a multi-lumen flexible polymeric extrusion having round lumens, first and second eyelets as well as cutting and pursing cable segments shown in FIG. 9;

FIG. 12 is a partial sectional view of distal end of single-use tissue incision and retrieval assembly seen in FIG. 2;

FIG. 13 is a is a perspective view of blade subassembly seen in FIG. 12 FIG. 14 is a side view of blade seen in FIGS. 12 and 13;

FIG. 15 is an end sectional view of tip region of single-use tissue incision and retrieval assembly taken through the plane 15-15 seen in FIG. 12;

FIG. 15A is a detailed end sectional view of multi-lumen flexible polymeric extrusion assembly seen in FIG. 15;

FIG. 16 is a partial plan view of a leaf member employed with the structure shown in FIG. 21 as it appears prior to the bending of its tip portion;

FIG. 16A is a partial view of the leaf shown in FIG. 16 with its tip bent into an operative orientation;

FIG. 16B is a partial plan view of a leaf member incorporating a multiplicity of slots as employed with the structure shown in FIG. 21 as it appears prior to the bending of its tip portion;

FIG. 17 is a sectional view of multi-lumen flexible polymeric extrusion assembly seen in FIG. 15 showing first and second electrically and thermally conductive leads with eyelets at their distal ends and the distal ends of the first and second tensionable portions of cutting and pursing cable as well as proximal ends of the resistively heated portion of the cutting and pursing cable;

FIG. 18 is a sectional view of multi-lumen flexible polymeric extrusion assembly showing first and second oval lumens and third and fourth round lumens having oval lumens positioned at the central portion of the extrusion;

FIG. 19 is a plan view of flat electrically and thermally conductive wire with eyelet at distal end;

FIG. 19A is a perspective view of multi-lumen flexible polymeric extrusion assembly showing first and second legs of electrically conductive wires with eyelets at their distal ends and the distal ends of the first and second tensionable portions of cutting and pursing cable wherein first tensionable portion of cutting and pursing cable passes through eyelet at distal end of second flat electrically and thermally conductive wire and second tensinable portion of cutting and pursing cable passes through eyelet at distal end of first flat electrically and thermally conductive wire as well as proximal ends of the resistively heated portion of the cutting and pursing cable;

FIG. 19B is a top view of multi-lumen flexible polymeric extrusion assembly showing first and second legs of electrically conductive wires with eyelets at their distal ends and the distal ends of the first and second tensionable portions of cutting and pursing cable wherein first tensionable portion of cutting and pursing cable passes through eyelet at distal end of second flat electrically and thermally conductive wire and second tensinable portion of cutting and pursing cable passes through eyelet at distal end of first flat electrically and thermally conductive wire as well as proximal ends of the resistively heated portion of the cutting and pursing cable;

FIG. 19C is sectional end-view of tissue incision and retrieval assembly taken through plane 19C-19C seen in FIG. 4;

FIG. 20 is a partial side sectional view of leaf member and extrusion assembly support member, drive tube and support tube seen in FIGS. 20A and 21;

FIG. 20A is a sectional view of leaf member and extrusion assembly support member taken through the plane 20A-20A seen in FIG. 20;

FIG. 21 is an assembly view of leaf member and extrusion assembly support member including leaf members and multi-lumen flexible polymeric extrusion;

FIG. 22 is a perspective view of the tip component of the single-use tissue cutting and capture assembly seen in FIG. 12;

FIG. 23 is a bottom view of the tip component of the single-use tissue cutting and capture assembly seen in FIG. 12;

FIG. 24 is a graph relating time with level of applied constant current to resistively heated portion of cutting and pursing cable as well as levels of cutting cable resistance during deployment of the tissue cutting and capture assembly according to the invention;

FIG. 25 is sectional view of distal end of tissue cutting and capture assembly showing path of tissue capture basket and blade at tip of tissue cutting and capture assembly;

FIG. 26 is a sectional view of single-use tissue cutting and capture assembly seen in FIG. 2 showing positions of drive block, leaf member and extrusion assembly support member and cable mounting hub prior to insertion into tissue of patient and prior to deployment of tissue cutting and capture assembly;

FIG. 26A is a sectional view of single-use tissue cutting and capture assembly seen in FIG. 2 showing positions of drive block, leaf member and extrusion assembly support member and cable mounting hub after insertion into tissue of patient adjacent to target tissue volume and prior to deployment of tissue cutting and capture assembly;

FIG. 27 is a sectional view of single-use tissue cutting and capture assembly seen in FIG. 2 showing positions of drive block, leaf member and extrusion assembly support member and cable mounting hub after insertion into tissue of patient adjacent to target tissue volume and at the maximum opening of tissue cutting and capture assembly;

FIG. 28 is a sectional view of single-use tissue cutting and capture assembly seen in FIG. 2 showing positions of drive block, leaf member and extrusion assembly support member and cable mounting hub after insertion into tissue of patient adjacent to target tissue volume and at the completion of deployment and purse down of tissue cutting and capture assembly;

FIG. 29 provides a schematic of an ex vivo tissue cutting apparatus used to measure the temperature using resistance thermometry as well as calculate the heat flux dissipated from a constant-current heated wire during the cutting of a sample of ex vivo animal tissue;

FIGS. 30A-30E combine, as labeled thereon, to provide a flow chart describing the methodology of the invention; and

FIG. 31 a photograph of a side view of a captured target tissue volume following the completion of the deployment of the tissue cutting and capture assembly that emerges from the cannula distal end assembly disposed at distal end of the delivery cannula.

The drawings will be described in more detail below.

### DETAILED DESCRIPTION OF THE INVENTION

A predominate characteristic of the present disclosure resides in the employment of a single-use tissue incision and retrieval assembly in conjunction with a reusable, battery-powered handpiece assembly. In a preferred embodiment of the detailed description of the present disclosure that follows and by way example, the tissue cutting and capture assembly includes a multi-lumen flexible polymeric extrusion assembly and five leaf members oriented in a regular hexagon pattern wherein the multi-lumen flexible polymeric extrusion assembly and five leaf members are positioned at the vertices of the hexagon pattern. The tissue incision and retrieval assembly is configured with a forward portion that extends to a forwardly disposed delivery cannula distal end assembly which incorporates a mechanically sharp cutting blade. Targeted tumor or tissue along with adjacent healthy tissue is circumscribed and encapsulated by a leaf member and multi-lumen flexible polymeric extrusion assembly through the utilization of a resistively heated portion of cutting and pursing cable extending along the distal tip of each leaf member and multi-lumen flexible polymeric extrusion assembly. The resistively heated portion of cutting and pursing cable provides a thermal cutting effect by virtue of being resistively heated and maintained above a temperature threshold and heat flux level sufficient to effect thermal cutting of tissue. As the multi-lumen flexible polymeric extrusion assembly and five leaf members are advanced forwardly and radially outwardly into tissue, the resistively heated cutting and pursing cable effects the thermal cutting of tissue. Once the distal ends of the multi-lumen flexible polymeric extrusion assembly and five leaf members reach a practitioner-selected tissue diameter (e.g., 25 mm), then the handpiece assembly commences the application of a sufficient level of tension to the resistively heated portion of the cutting and pursing cable in combination with the continuing forward advancement of the multi-lumen flexible polymeric extrusion assembly and five leaf members to induce the pursing down of the distal ends of the multi-lumen flexible polymeric extrusion assembly and five leaf members. Upon completion of the pursing down of the distal ends of the multi-lumen flexible polymeric extrusion assembly and five leaf members, the incised tissue becomes circumscribed within a tissue capture basket. The forward portion of the tissue incision and retrieval system along with the tissue capture basket containing the excised volume of target tissue are then withdrawn from the body of the patient.

In a preferred embodiment, two of the four lumens within the multi-lumen flexible polymeric extrusion assembly serve as conduits for the first and second electrically and thermally conductive leads having eyelets at their distal ends. Also, in a preferred embodiment, the other two of the four lumens within the multi-lumen flexible polymeric extrusion assembly serve as conduits for the first and second tensionable portions of the cutting and pursing cable. By selecting a component orientation establishing where a pursing or constricting action commences, the maximum leading-edge periphery for capture may be elected and, typically, may range, for example, from about a 15 mm to about a 30 mm effective diametric extent.

Initial positioning of the delivery cannula tip in confronting adjacency with a tissue volume is facilitated through the utilization of a surgically sharp cutting blade assembly located at the tip. Following appropriate positioning of the tip, a motor-actuated drive tube drive member translation assembly is enabled to actuate the forward deployment of the multi-lumen flexible polymeric extrusion assembly and five leaf members wherein eyelets located at the distal ends of the multi-lumen flexible polymeric extrusion assembly and the leaf members support the resistively heated portion of the cutting and pursing cable as it incises and circumscribes the target tissue volume.

A desirable feature of the tissue incision and retrieval system of the present disclosure resides in the incorporation of the delivery cannula and cable-implemented leaf member and multi-lumen flexible polymeric extrusion assembly within a single-use support housing. That single-use support housing is removably mounted within a reusable handpiece assembly containing a first motor-actuated drive tube drive member translation assembly, a second motor-actuated cable mounting hub translation assembly, a control system and internal energy source. Hereinafter, the term energy source refers to a rechargeable battery and the two terms are equivalent. A practitioner-accessible capture size selection switch is located on the handpiece assembly for pre-selection of the diameter of a substantially spherical volume of tissue along with a practitioner-accessible display of the selected capture size enables the tissue excision and retrieval assembly to be used to incise and capture substantially spherical target tissue volumes (e.g., human breast tissue) having maximum diameter extents ranging from 15 to 30 mm. The term “cannula” or “delivery cannula” as used herein is intended to refer to any elongate surgical delivery structure, rigid or flexible, having a capability for deploying the resistively heated portion of the cutting and pursing cable.

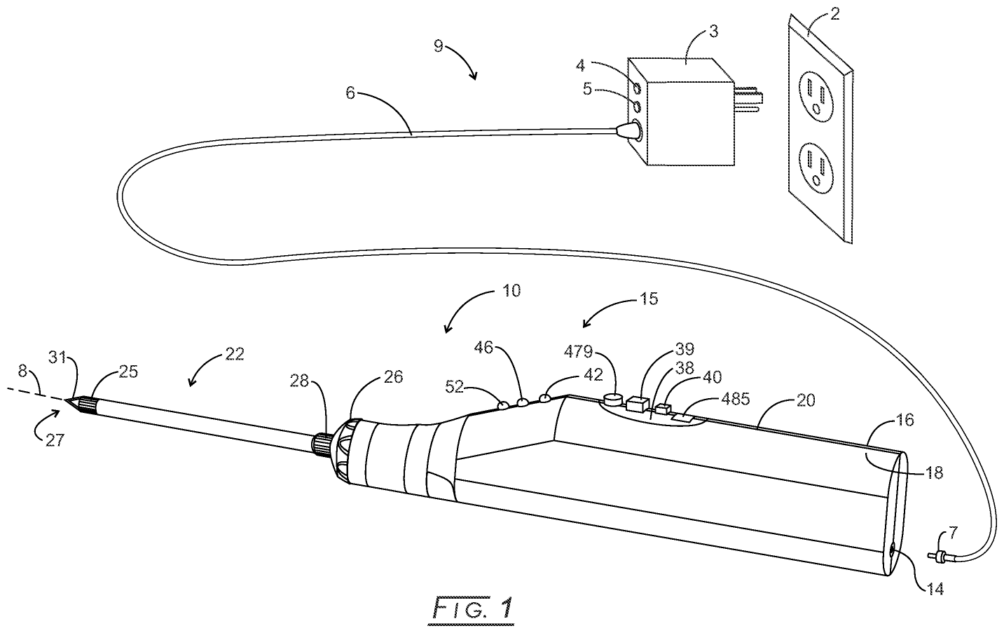

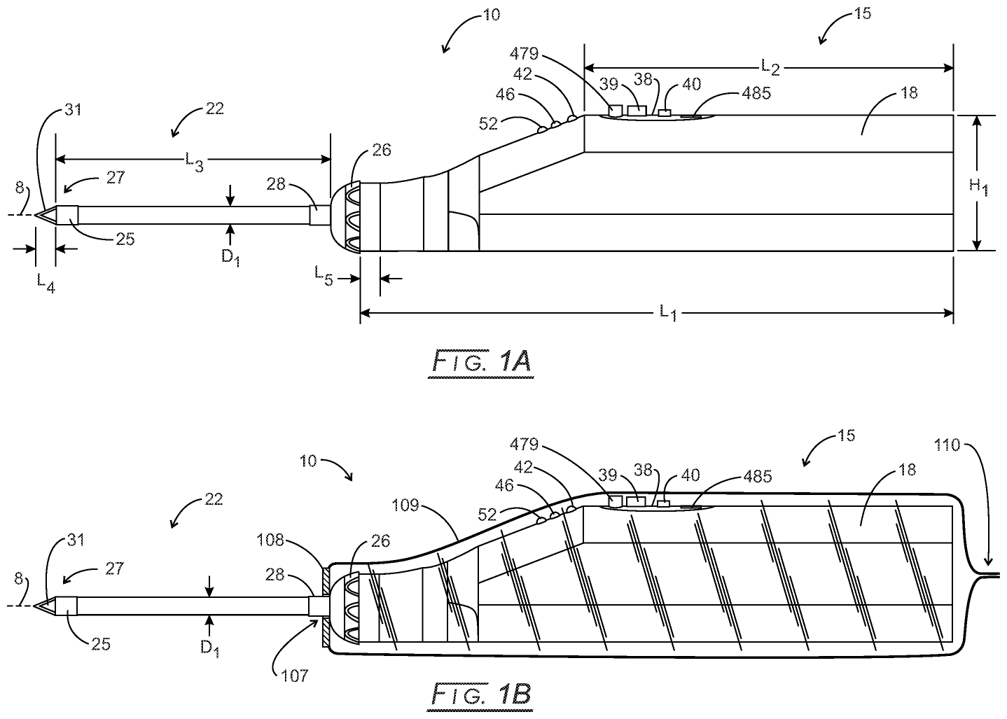

Referring to FIG. 1, a tissue incision and retrieval system according to the present disclosure is represented in general at 10. Tissue incision and retrieval system 10 includes a single-use tissue incision and retrieval assembly having an elongate delivery cannula 22 in combination with a reusable handpiece assembly represented generally at 15. By way of example, reusable handpiece assembly 15 may comprise a housing right side 16 and housing left side 18. Reusable handpiece assembly 15 may be formed of two molded components shown as housing right side 16 and housing left side 18. Housing sides 16 and 18 extend mutually outwardly from a medial plane represented at a joint line 20. An elongate delivery cannula represented at 22 is shown supported from the forward portion of the handpiece assembly 15, which extends along a longitudinal axis 8. A distal end of the delivery cannula 22 extends through a locking nut 26, which is retained in position by a collar 28. The forward end of the delivery cannula 22, as represented at 27 extends to a distal end or tip 25. Distal end or tip 25 also supports a surgically sharp blade 31, having thickness t2, which enables the initial advancement and positioning of the distal end of tip 25 of delivery cannula 22 in a confronting relationship with respect to the targeted tissue site (not shown).

Still referring to FIG. 1, practitioner actuatable on/off and initialization switch 40 and start incision and capture switch 39 are incorporated within a switch housing 38. The on/off and initialization switch 40 and start incision and capture switch 39 are in communication with a control system (not shown) within handpiece assembly 15 via lead wires and/or printed circuit board lead traces. Practitioner actuatable capture size selection switch 479 and selected capture size display 485 are also incorporated within a switch housing 38.

The tissue incision and retrieval system 10 also includes a handpiece assembly battery charger 9 that comprises an alternating current to direct current converter 3, first battery charger indicator light 4 used to provide a visual cue that handpiece assembly battery recharging is in progress, second battery charger indicator light 5 used to provide a visual cue that handpiece assembly battery recharging has been completed, handpiece assembly battery charging cable 6 and handpiece assembly battery charging cable connector 7 removably attachable to charging receptacle 14 on handpiece assembly 15.

Still referring to FIG. 1, upon depressing the on/off and initialization swtich 40, the controller (not shown) within the handpiece assembly 15 tests the energy capacity of the rechargeable battery located within handpiece assembly 15. When on/off switch 40 is depressed and the battery energy capacity level is determined to be sufficient for a tissue cutting and capture procedure, a visually accessible on/off and “Ready” indicator light 42 distal to on/off switch 40 on handpiece assembly 15 is illuminated. By way of example, on/off and “Ready” indicator light 42 may be a yellow light emitting diode (LED). If the energy capacity of the rechargeable battery within handpiece assembly 15 is insufficient to perform a tissue cutting and capture procedure, then all three display lights will flash repeatedly until the on/off and initialization switch 40 is again depressed indicating that the recharging of the rechargeable battery within handpiece assembly 15 using handpiece assembly battery charger 9 is required.

Upon illumination of the on/off and “Ready” indicator light 42 indicating sufficient energy stored in rechargeable battery within handpiece assembly 15, the practitioner next selects the maximum diameter of the intended tissue capture volume by depressing capture size selection switch 479 and observing selected capture size at selected capture size display 485. The practitioner next inserts the forward end 27 of delivery cannula 22 into tissue of patient's body aided by blade 31 and advances the forward end 27 of delivery cannula 22 into confronting adjacency to target tissue volume aided, for example, by ultrasound, stereotactic radiography or computer-based robotic guidance methods. Once in confronting adjacency with respect to the target tissue volume, the start tissue incision and capture switch 39 is momentarily depressed. Upon depressing the start tissue incision and capture switch 39, a visually accessible capturing indicator light 46, located distal to the start tissue incision and capture switch 39, on handpiece assembly 15 is singularly illuminated. By way of example, capturing indicator light 46 may be a green light emitting diode (LED). In addition to a visually accessible capturing indicator light 46, an audible cue may be generated within the handpiece assembly 15 that continues throughout the period of tissue incision and capture procedure.

Upon completion of the tissue incision and capture procedure, as determined by the controller within the handpiece assembly 15, based on the measured electrical impedance of that portion of the heatable cutting and pursing cable conducting the applied constant current, the capture complete indicator light 52 is singularly illuminated, the applied constant current and the applied voltage to the first motor-actuated drive tube drive member translation assembly is suspended, the second motor-actuated cable mounting hub translation assembly and the audible cue is suspended. At the completion of the tissue incision and capture procedure, the practitioner withdraws the distal end portion of delivery cannula 22 from the patient, which includes the captured and substantially spherical tissue volume.

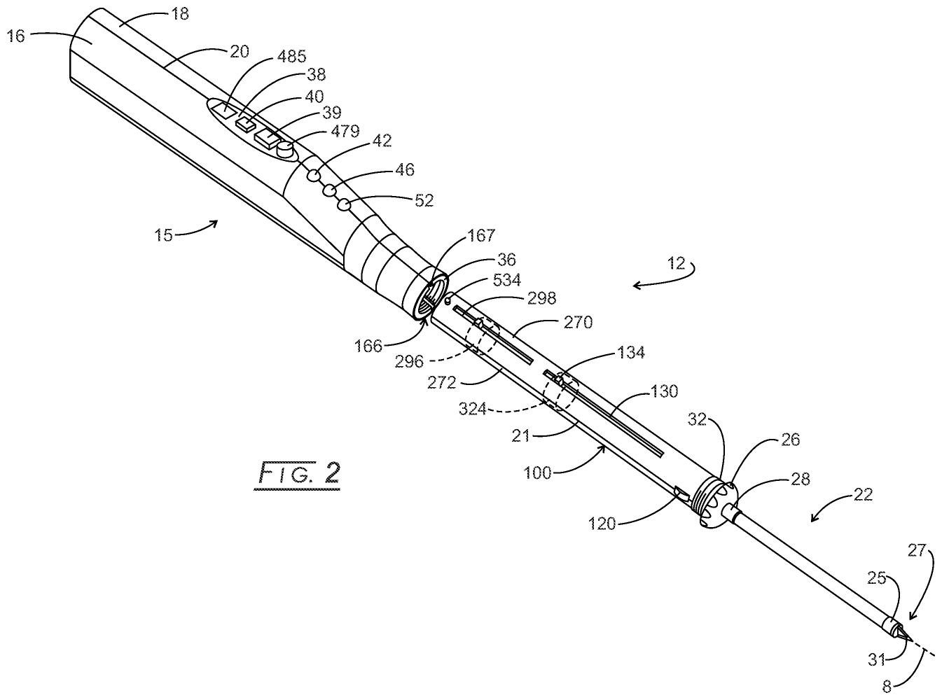

Referring to FIGS. 1 and 2, the single-use tissue incision and retrieval assembly 12 inserted in handpiece assembly 15 is indicated generally at 10. Referring to FIG. 2, single-use tissue incision and retrieval assembly 12 is revealed in an orientation prior to insertion within the reusable handpiece assembly 15. As seen in FIG. 2, delivery cannula 22 is seen extending forwardly from a cylindrically shaped single-use support housing 100. The forward end of single-use support housing 100 supports locking nut 26. In this regard, it may be observed that locking nut 26 is configured with an external groove and keyway slot that engages with and is secured by distal end of reusable handpiece assembly 15 (not shown in FIG. 2 but seen in FIG. 3).

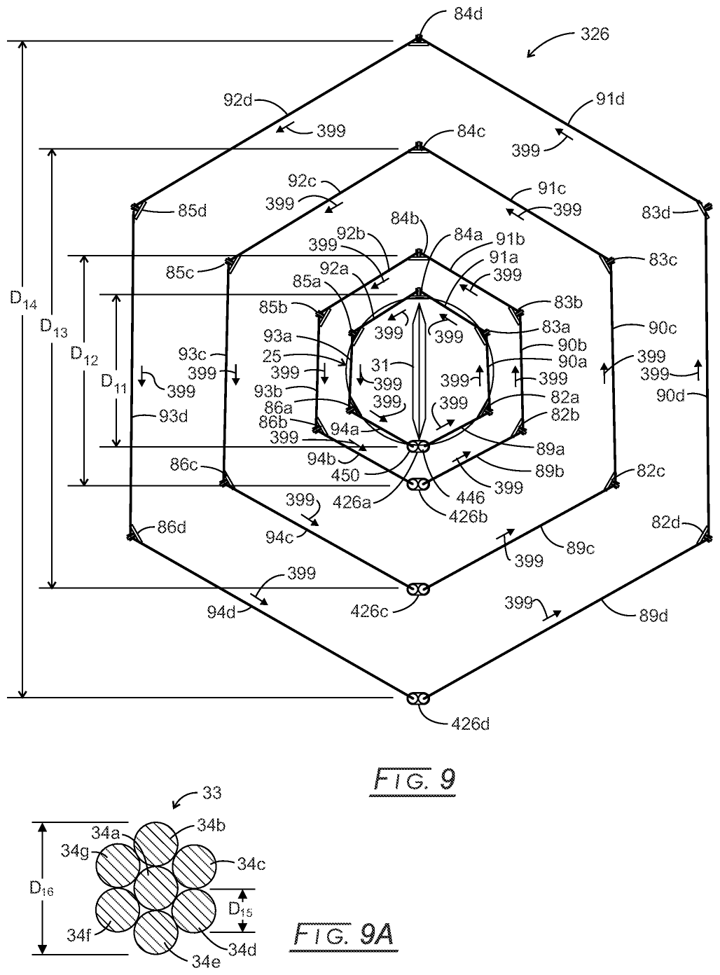

In the specifications that follow and seen in FIGS. 9 and 17, a substantially constant electrical current level operating at an elevated frequency of at least 25 kHz and preferably 100 KHz follows electrical current path 399 through first support housing lead wire 116 to first electrically and thermally conductive lead 444 and to first electrically and thermally conductive eyelet 446 and transferred to first resistively heated portion of the cutting and pursing cable 89 at sliding electrical contact between first electrically and thermally conductive eyelet 446 and first resistively heated portion of cutting and pursing cable 89. As seen in FIGS. 9 and 17, the constant current continues sequentially along path 399 through first resistively heated portion 89 and continuing to sixth resistively heated portions of cutting and pursing cable 94. The constant current is transferred from the sixth resistively heated portions of cutting and pursing cable 94 to electrically and thermally conductive lead 448 through sliding electrical contact between sixth resistively heated portions of cutting and pursing cable 94 and electrically and thermally conductive eyelet 450. Electrical current path 399 continues through second electrically and thermally conductive lead 448 to second support housing lead wire 114. The applied constant current is confined to the first through sixth segments of the resistively heated portion of the cutting and pursing cable 89 to 94, respectively, wherein first resistively heated portion 89 extends distal to first electrically and thermally conductive eyelet 446 and sixth resistively heated portion 94 extends distal to second electrically and thermally conductive eyelet 450. In this manner, a constant current level flows only to first through sixth segments of the resistively heated portion of the cutting and pursing cable 89-94 that are distal to first and second electrically and thermally conductive eyelets 446 and 450 and only those portions that are in in contact with tissue 366.

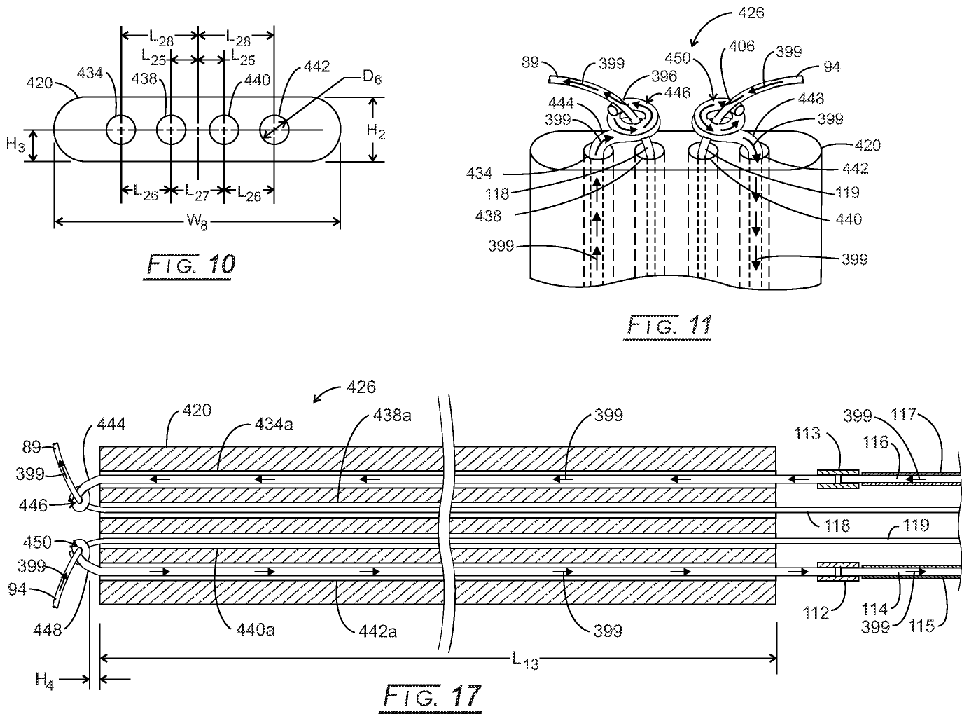

Referring now to FIGS. 2, 3, 8, 11 and 17, positioned at the distal end of support housing 100 are two spaced apart first and second electrical contacts 120 and 122 (seen in FIG. 8) which are oriented to make contact with corresponding first and second electrical terminals 186 and 188 disposed within reusable handpiece assembly 15 upon insertion of single-use support housing 100 within the receiving cavity 166. First and second electrical contacts 120 and 122 serve as two electrical poles or electrodes that selectively receive constant current flowing along electrical current flow paths 399x and 399z (seen in FIG. 3), the applied constant current preferably being an alternating current having a frequency in the range from 25 kHz to 300 kHz, preferably 100 KHz. The constant current is conducted through first and second electrically and thermally conductive leads 444 and 448 to first and second electrically and thermally conductive eyelets 446 and 450, respectively. Sliding electrical contact between [a] first and second tensionable portions of cutting and pursing cables 118 and 119 and [b] first and second electrically and thermally conductive eyelets 446 and 450, respectively, conducts the constant current into the first and sixth resistively heated portions of the cutting and pursing cables 89 and 94, respectively, beginning at first and second transition boundaries 396 and 406, respectively, as seen in FIG. 11.

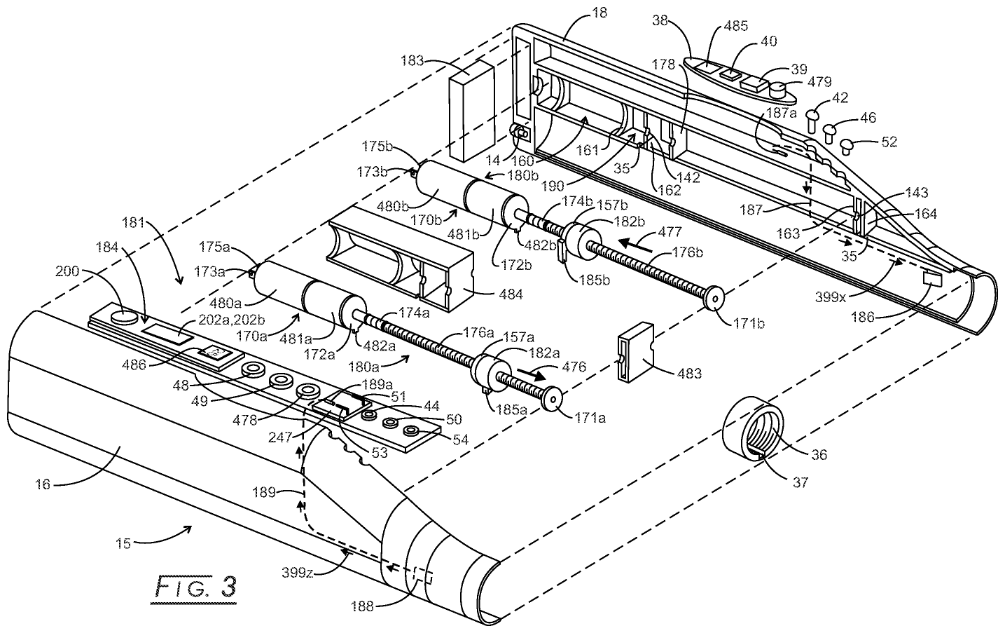

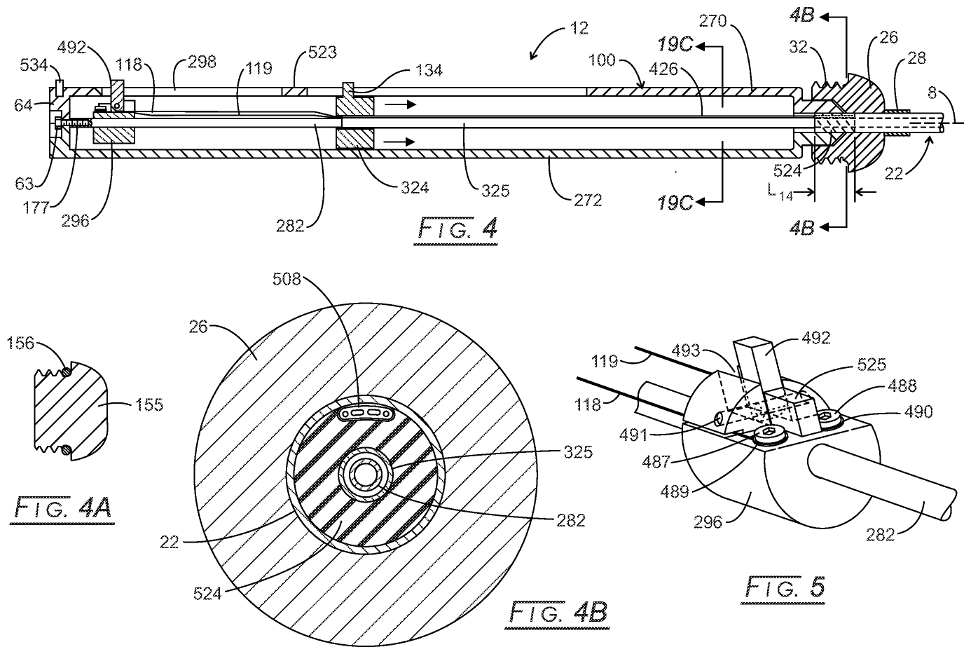

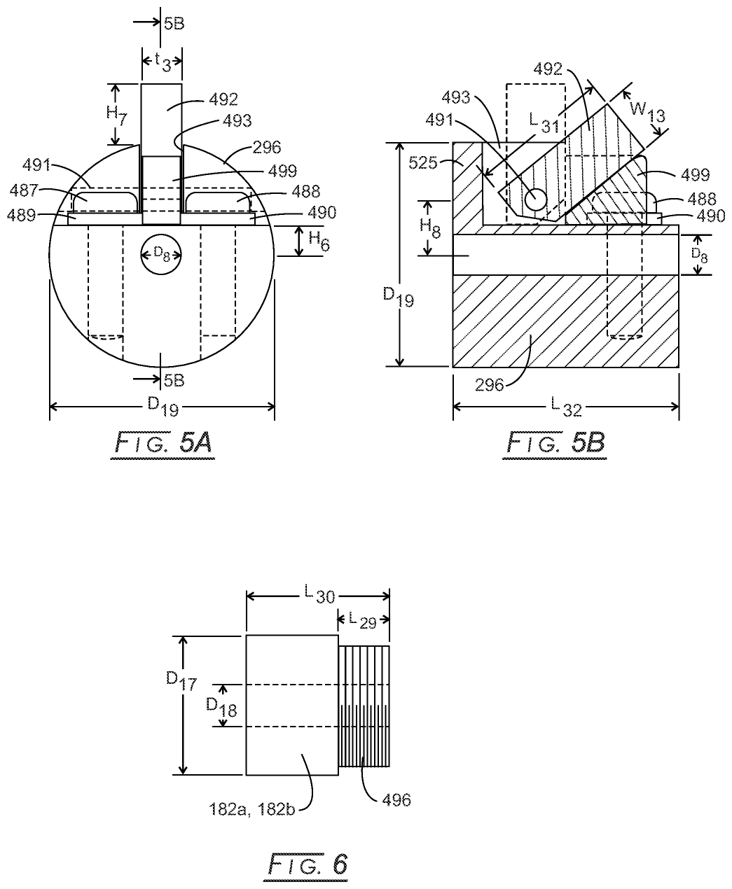

Turning now to FIGS. 4, 5 and 8, the first and second tensionable portions of cutting and pursing cables 118 and 119 extend rearwardly and are secured to cable mounting hub 296 (seen in phantom in FIG. 8) by first and second cable fastening screws 488 and 489, respectively, as seen in FIG. 5.

Referring to FIGS. 2, 4, and 5, the tensionable portions of cutting and pursing cables 118, 119, extend rearwardly to cable mounting hub 296 having a protruding pivotable cable mounting hub advancement drive ear 492 that slideably translates within first elongate stabilization slot 298 arranged in parallel with axis 8.

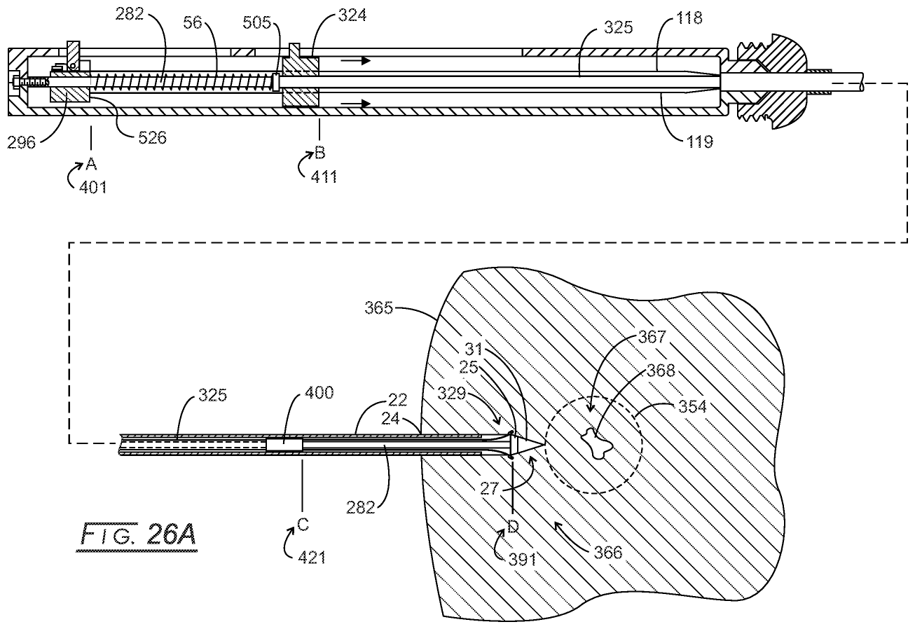

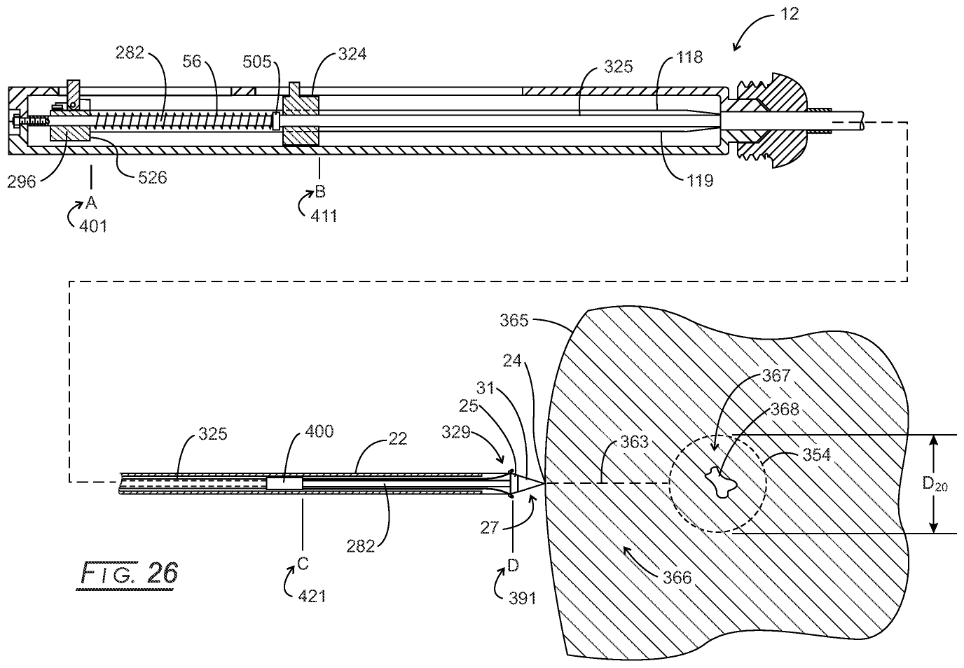

Referring now to FIGS. 2, 3, 4 and 8, second elongate stabilization slot 130 is found on the same side of the upper half of single-use casing 270 that forms support housing 100. The second elongate stabilization slot 130 is similarly arranged in parallel with axis 8. An outwardly extending drive block advancement ear 134 protrudes from drive assembly drive member 324 through second elongate stabilizer slot 130. The drive block advancement ear 134 is engaged by rearwardly disposed first pivotable drive finger 185a supported by translation nut 182a as seen in FIG. 3. The driven surface of drive block advancement ear 134 is used to impart forward movement to drive assembly drive member 324 functioning, in turn, to deploy the ensemble of leaf members and the multi-lumen flexible polymeric extrusion assembly 400 (as seen in FIG. 21) from delivery cannula 22 (as seen in FIGS. 26-28).

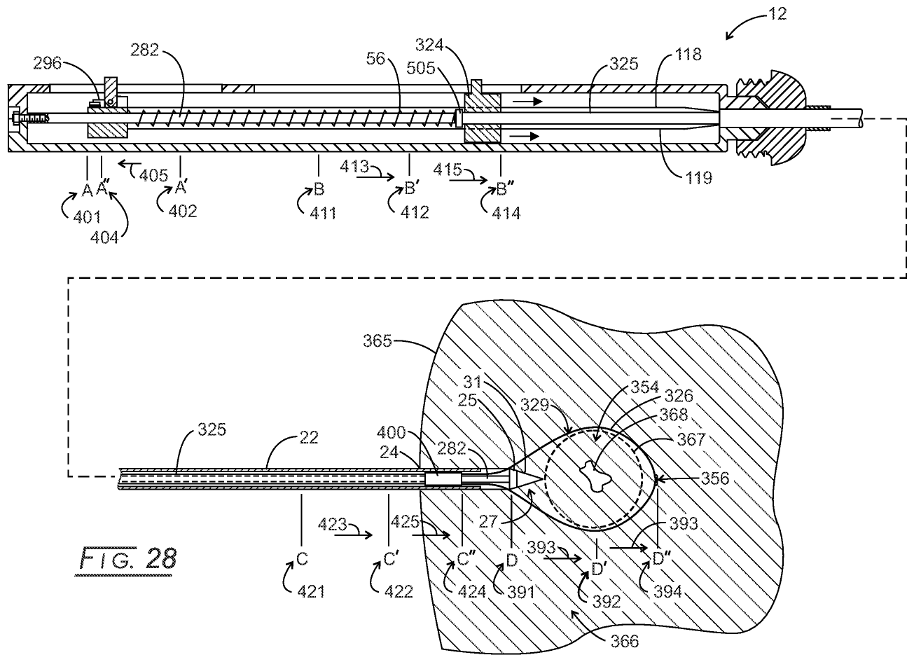

Referring now to FIGS. 2, 3, 4 and 5, when the support housing 100 of single-use tissue incision and retrieval assembly 12 is inserted into the receiving cavity 166 of reusable handpiece assembly 15, guide pin 534 ensures proper alignment of single-use tissue incision and retrieval assembly 12 within the receiving cavity 166 of reusable handpiece assembly 15, thereby enables the passage of the drive block advancement ear 134 through oppositely disposed alignment key notch 167 in threaded insert 36 and along third elongate slot 35 in handpiece assembly 15 until it is positioned just proximal to first pivotable drive finger 185a as the support housing becomes fully inserted into handpiece assembly 15. Likewise, pivoting cable mounting hub advancement drive ear 492 passes through oppositely disposed alignment key notch 167 in threaded insert 36 and continues to be advanced along third elongate slot 35 in handpiece assembly 15 until it is positioned just proximal to second pivotable drive finger 185b as the support housing becomes fully inserted into handpiece assembly 15. However, in order for the pivotable cable mounting hub advancement drive ear 492 to be advanced rearwardly beyond the protruding first pivotable drive finger 185a, the cable mounting hub advancement drive ear 492 needs to pivot to allow passage past protruding first pivotable drive finger 185a. An internal spring (not shown) within cable mounting hub 296 returns pivotable cable mounting hub advancement drive ear 492 to its vertical orientation as seen in FIG. 5 as required to drive Cable mounting Hub rearwardly during the pursing down of the multi-lumen flexible polymeric extrusion assembly 426 and five leaf members 82-86 as seen in FIG. 28. First and second pivotable drive fingers 185a and 185b are pivotable to enable both the insertion and removal of the support housing 100 from the handpiece assembly in the presence of the protruding drive block advancement ear 134 and the cable mounting hub advancement drive ear 492. Upon completion of the purse down of the multi-lumen flexible polymeric extrusion assembly 426 and five leaf members 82-86, second motor-actuated cable mounting hub translation assembly 180b advances forwardly until second pivotable drive finger engages body of support housing 100 at the distal end 523 of first elongate stabilizing slot 298 as seen in FIG. 4. By this engagement at the end of the tissue capture procedure, the second pivotable drive finger 185b is pivoted to the extent that there is no interference with the cable mounting hub advancement drive ear 492 as the support housing 100 is withdrawn from the handpiece assembly 15.

Referring to FIG. 3, the handpiece assembly 15 is revealed in exploded fashion. As seen in FIG. 3, the exterior surface of housing right side 16 of reusable handpiece assembly 15 is revealed and the corresponding interior of housing left side 18 is revealed. These two sides are symmetrical and contain the usable components described in greater detail below. The housing right side 16 and housing left side 18 are formed with one half of second motor and planetary gear train assembly 170b mount chambers as shown at 160 and 190 in connection with housing left side 18. Positioned just forwardly of the chamber 160 are first and second bulkheads 161 and 162, respectively. The bulkhead 162 defining first circular openings 142 to support the proximal end of second lead screw 176b. Third bulkhead 163 defines second circular opening 143, supporting the distal end of second lead screw 176b. A forward cavity of housing right side 16 and housing left side 18 in the space between the third bulkhead 163 and fourth 164 are each configured with an opening to contain thrust bearing 171b as represented in connection with housing left side 18.

Still referring to FIG. 3, positioned within motor mount chamber 160 is second motor and planetary gear assembly represented generally at 170b, which incorporates motor component 480b in combination with planetary gear assembly 481b. By way of example, second motor and planetary gear assembly 170b may comprised of a D.C. motor 480b having, by way of example, a 3.2 watt assigned power rating marketed under the catalog designation 118686 by Maxon Precision Motors Inc., of Burlingame, Calif. This motor 480b is combined with a planetary gear 481b exhibiting, by way of example, a 29:1 reduction and marketed under the catalog designation 118185a by Maxon Precision Motors Inc. (supra). The motor and planetary gear assembly 170b is relatively securely positioned within chamber 160 to the extent that it has limited freedom of rotational movement except for the axial drive pin (not shown) at the distal end of planetary gear assembly 170b. In this regard, a torque stop component key 482b prohibiting overall motor assembly rotation is coupled to the forward or output end of motor and planetary gear assembly 481b.

Still referring to FIG. 3, the mechanical output from motor and planetary gear assembly 170b is connected through metallic flexible bellows-shaped coupler 174b extending between second bulkhead 162 and fifth bulkhead 178 to connection with second lead screw 176b implemented with the threaded elongate channel of a second translation nut 182b arranged in parallel with the longitudinal axis 8 of the tissue incision and retrieval assembly 12. The metallic flexible bellows-shaped coupler bellows 174b provides a torsionally rigid, but axially flexible coupling that reduces the vagaries of elongate mechanical-rotational force transmission. By way of example, bellows coupler as at 174b is marketed under a model designation SC-3 by Servometer Corp. of Cedar Grove, N.J. Alternatively, other flexible coupling components may be used for this purpose including a helical beam coupler marketed by Helical Products Company, Santa Maria, California. By way of example, second lead screw as at 176b is marketed under the designation term “lead” by Thomson Linear of Radford, Virginia.

Still referring to FIG. 3, rotatably driven from motor and planetary gear assembly 170b through bellows-shaped coupler 174b, the distal end of lead screw 176b is supported and rotatable within thrust bearing 171b located between bulkheads 163 and 164. With this arrangement, a freedom of rotational movement is provided for the entire assembly proximal to second circular opening 143 including bellows-shaped coupler 174b and second lead screw 176b permitting the motor and planetary gear assembly 170b to be mounted in self aligning confinement within the motor mount chamber housing 160. Thus, binding or like phenomena are avoided in connection with the motor drive actuator system. Second lead screw 176b is threadably engaged with a second motor-actuated cable mounting hub translation assembly represented generally at 180b which comprises a translation nut 182b and a second pivotable drive finger 185b which is configured to extend to a position separated from but aligned for driven engagement with the cable mounting hub advancement drive ear 492 (as seen in FIGS. 2 and 4) after the support housing 100 is inserted in the receiving cavity 166 of handpiece assembly 15. By way of example, translation nut component as at 182b is marketed under the designation translation nut, ball nut and SuperNut by Thomson Linear of Radford, Virginia.

Still referring to FIG. 3 and in like manner, the housing right side 16 is symmetrically configured to housing left side 18 and is formed with one half of motor and planetary gear train assembly mount chambers (not shown) similar to chambers shown at 160 and 190 in connection with housing left side 18 along with bulkheads, circular openings and opening to contain thrust bearing 171a and support first lead screw 176a as previously disclosed with regard to housing left side 18 in FIG. 3. Positioned within motor mount chamber located in the housing right side 18 (not shown) is motor and planetary gear assembly represented generally at 170a, which incorporates motor component 480a in combination with planetary gear assembly 481a. By way of example, first motor and planetary gear assembly 170a may comprised of a D.C. motor 480a having, by way of example, a 3.2 watt assigned power rating marketed under the catalog designation 118686 by Maxon Precision Motors Inc., of Burlingame, Calif. This motor 480a is combined with a planetary gear 481a exhibiting, by way of example, a 29:1 reduction and marketed under the catalog designation 118185a by Maxon Precision Motors Inc. (supra). The motor and planetary gear assembly 170a is relatively securely positioned within its motor mount chamber (not shown) to the extent that it has limited freedom of rotational movement except for the axial drive pin (not shown) at the distal end of planetary gear assembly 170a. In this regard, a torque stop component key 482a prohibiting overall motor assembly rotation is coupled to the forward or output end of motor and planetary gear assembly 481a.

Still referring to FIG. 3, the mechanical output from motor and planetary gear assembly 170a is connected through metallic flexible bellows-shaped coupler 174a extending between closely spaced bulkheads (not shown) to connection with first lead screw 176a implemented with the threaded elongate channel of a second translation nut 182a arranged in parallel with the longitudinal axis 8 of the tissue incision and retrieval assembly 12. The metallic flexible bellows-shaped coupler bellows 174a provides a torsionally rigid, but axially flexible coupling that reduces the vagaries of elongate mechanical-rotational force transmission.

Still referring to FIG. 3, rotatably driven from motor and planetary gear assembly 170a through bellows-shaped coupler 174a, the distal end of first lead screw 176a is supported and rotatable within thrust bearing 171a located between closely spaced bulkheads (not shown). With this arrangement, a freedom of rotational movement is provided for the entire assembly proximal to circular opening including bellows-shaped coupler 174a and first lead screw 176a permitting the motor and planetary gear assembly 170a to be mounted in self aligning confinement within the motor mount chamber housing (not shown). Thus, binding or like phenomena are avoided in connection with the motor drive actuator system. First lead screw 176a is threadably engaged with a motor-actuated drive tube drive member translation assembly represented generally at 180a which comprises a translation nut 182a and a second pivotable drive finger 185a which is configured to extend to a position spaced from but aligned for driven engagement with the drive block advancement ear 134 (as seen in FIGS. 2 and 4) after the support housing 100 is inserted in the receiving cavity 166 of handpiece assembly 15.

Still referring to FIG. 3, constant current source 247 is integrated within circuit board assembly 184 with first and second leads 187 and 189 extending between [a] first and second constant current source connector pins 51 and 53, respectively and [b] first and second electrical terminals 186 and 188, respectively. First and second electrical terminals 186 and 188 are mounted on the interior surface of housing left side 18 and housing right side 16, respectively, at the distal or forward end of receiving cavity 166 and may, by way of example, be retained in place by an adhesive. The first and second electrical terminals 186 and 188 supply constant current to the mating contact surfaces of corresponding first and second electrical contacts 120 and 122, respectively, located on the single-use support housing 100 (as seen in FIG. 8).

Still referring to FIG. 3, internal energy source and control system 181 is contained within handpiece assembly 15 and includes, by way of example, rechargeable battery 183 and circuit board assembly 184. By way of example, the circuit board assembly 184 comprises [a] one or more microcomputers 202 having machine instructions to control tissue incision and capture functions including timing and level of applied constant current and constant voltage in response to on/off and initialization switch 40 and practitioner actuated start tissue incision and capture switch 39 located on handpiece assembly 15, [b] machine instructions within microcomputer 202 to control illumination of display components on handpiece assembly 15 including “Ready” indicator light 42, “Capturing” indicator light 46 and “Capture Complete” indicator light 52, [c] constant current source 247, [d] constant voltage source (not shown) for energizing and controlling speed of motors 170a and 170b, [e] first switch sensor 48, [f] second switch sensor 49, [g] third switch sensor 478, [h] first light emitting diode 44, [i] second light emitting diode 50, [j] third light emitting diode 54, [k] first constant current source connector pin 51, [I] second constant current source connector pin 53 [m] numerical capture size display 486 and [n] machine instructions within microcomputer 202 to continuously monitor the electrical resistance of the resistively heated cutting and pursing cable circuit and briefly interrupt the application of constant current if the measured resistively heated cutting and pursing cable circuit resistance increases greater than a pre-determine increase limit during a pre-selected time interval (e.g., 0.002 seconds).

The constant current source 247 supplies constant current to the first through sixth segments of the resistively heated portion of the cutting and pursing cable 89-94, as described in greater detail in the specification that follows. Circuit board assembly 184 includes [a] circuitry and logic to respond to first, second and third sensor switches 48, 49 and 479, [b] circuitry and logic to control illumination of light emitting diodes 44, 50, 54, [c] circuitry to display selected size (diameter) of target tissue volume 354 (as seen in FIG. 28) and [d] circuitry as well as logic to control the start of advancement as well as the termination of advancement of the leaf members and multi-lumen flexible polymeric extrusion assembly 400 (as seen in FIG. 21) during the process of tissue incision and capture. Rechargeable battery 183 provides source of DC voltage and current to the constant current source 247 incorporated in circuit board assembly 184. The rechargeable battery 183 is recharged by attaching an external source of DC power to charging receptacle 14 shown, by way of example, at the proximal end of handpiece assembly. Off/on and initialization switch 40 and start tissue incision and capture switch 39 mounted on waterproof switch housing 38 activate first and second sensor switches 48 and 49, respectively, when manually depressed by the practitioner. Prior to the start of tissue incision and capture, capture size selection switch 479 activates third switch sensor 478 each time it is depressed by the practitioner with a corresponding increment in the numerical capture size display 486 and visible through capture size display window 485 (as seen in FIGS. 1, 1A and 1B).

Advantageously, all penetrations through the left and right housing sides 16 and 18 including waterproof switch housing 38, light emitting diodes 44, 50, 54 and charging receptacle 14 as well as the joining together of the left and right housing sides 16 and 18 are assembled with water-tight seals. In addition, as seen in FIGS. 4 and 4B, a liquid barrier seal 524 is positioned at the proximal end and within delivery cannula 22 to prevent the ingress of body fluids that may enter at the forward end 27 of delivery cannula 22 from reaching the interior of the tissue incision and retrieval assembly 12 and spreading further into and thereby contaminating the interior of the reusable handpiece assembly 15.

During cleaning and disinfecting operations performed between uses of the reusable handpiece assembly 15, the temporary placement of sealing cap 155 in combination with O-ring 156 (as seen in FIG. 4A) form a further water-tight seal at the distal end of handpiece assembly 15.

Referring to FIG. 4, a sectional view is presented illustrating the operative association of the motor drive features with the single-use support housing 100 contained components. As seen in FIGS. 3 and 4, second motor and planetary gear assembly 170b is seen to be located adjacent to motor mount chamber 160. As noted above, within motor mount chamber 160, second motor and planetary gear assembly 170b is permitted some self-aligning movement but is restrained from rotational movement by torque stop component 172b. The output from the planetary gear assembly 481b is coupled to the driven input side of bellows-shaped coupler 174b which is seen to extend through coupler cavity 190 defined by oppositely disposed and spaced apart second bulkhead 162. The elongate threaded second lead screw 176b is seen extending from first circular opening 142 in second bulkhead 162. Second bulkhead 162 and associated first circular opening 142 provide support against all of the driving forces imposed from the second motor and planetary gear assembly 170b as it drives the second translation nut 182b along the length of second lead screw 176b. Likewise, as seen in FIGS. 3 and 4, first motor and planetary gear assembly 170a is seen to be located adjacent to double-sided first and second motor and planetary gear assembly and retaining cover 484 for first and second lead screws 176a, 176b. Within the corresponding motor mount chamber for the first motor and planetary gear assembly 170a, some self-aligning movement is permitted but is restrained from rotational movement by torque stop component 172b. The output from the planetary gear assembly 481a is coupled to the driven input side of bellows-shaped coupler 174a which extends through coupler cavity defined by oppositely disposed and spaced apart second bulkhead (not shown). A second bulkhead and associated circular opening (not shown) provide support against all of the driving forces imposed from the first motor and planetary gear assembly 170a as it drives the first translation nut 182a along the length of the first lead screw 176a.

The exploded view seen in FIG. 3 combined with the cross-sectional view in FIG. 4 reveals that the first pivotable drive finger 185a is configured to engage drive block advancement ear 134 to urge drive assembly drive member 324 in forward direction 476. As drive assembly drive member 324 is driven in forward direction 476, the drive tube 325 attached to drive assembly drive member 324 is likewise driven forwardly. A seen in FIGS. 4 20 and 21, the drive tube 325 is attached to leaf member and extrusion assembly support member 347, thereby driving the leaf members 82-86 and multi-lumen flexible polymeric extrusion assembly 426 forwardly as drive assembly drive member 324 is driven forwardly by first pivotable drive finger 185a extending from the first drive nut 182a of the first motor-actuated drive tube drive member translation assembly 180a.

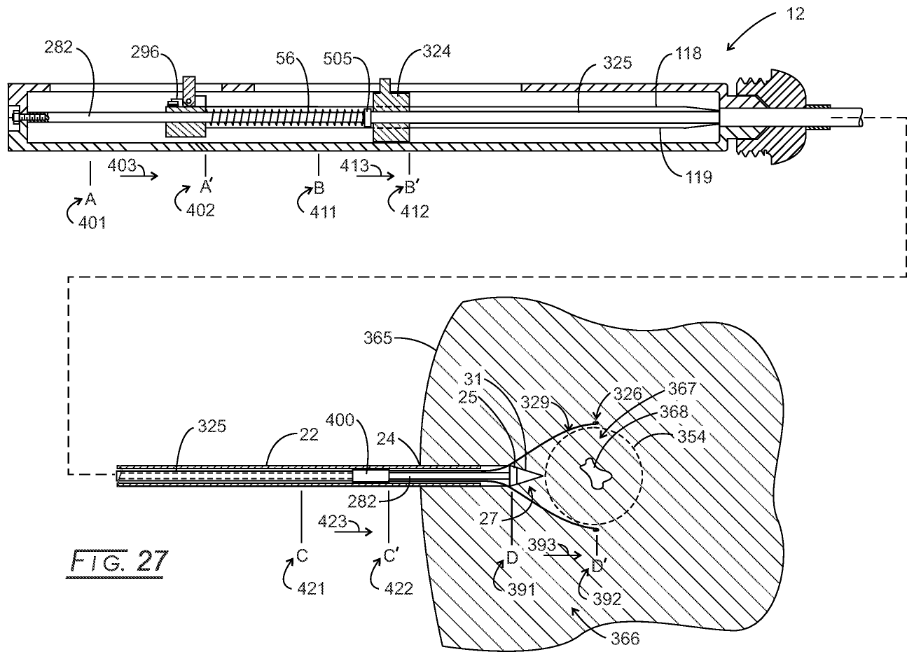

Likewise, the exploded view seen in FIG. 3 combined with the cross-sectional views in FIGS. 4 and 27 reveals a second pivotable drive finger 185b that extends from the second drive nut 182b of the second motor-actuated drive tube drive member translation assembly 180b. The second pivotable drive finger 185b is configured to engage the cable mounting hub assembly 296 and is pre-positioned by the practitioner's selection of the maximum diameter of the target tissue volume to be captured. The pre-positioned location of the second pivotable drive finger 185b stops the forward movement of cable mounting hub assembly 296 thereby constraining the maximum opening of the tissue cutting and capture assembly 329 as seen at position 392 in FIG. 27. Once the maximum opening of the tissue cutting and capture assembly 329 is reached, second pivotable drive finger 185b is driven in rearward direction 477 by the second motor-actuated cable mounting hub translation assembly 180b at a predetermined rate. As the cable mounting hub assembly 296 is driven in rearward direction 477, the increased tension in the tensioning portions of the cutting and pursing cable 118, 119 as well as the first through sixth segments of the resistively heated portion of the cutting and pursing cable 89-94 causes the eyelet containing tips of leaf members 82-86 and multi-lumen flexible polymeric extrusion assembly 426 to be drawn together in a pursing manner as seen in FIG. 28 at position 394 corresponding to the end of tissue cutting and capture.

Returning momentarily to FIG. 2, some details of the forward end 27 of delivery cannula 22 are also revealed. The forward end 27 is depicted as it is utilized for the incision and capture of tissue volumes, for example, encompassed within a diametric extent of about 30 mm. The tip incorporates a surgically sharp blade 31 positioned with the blade tip coincident with longitudinal axis 8.

Referring now to FIGS. 1B and 3, the incorporation of the energy source and control system 181 into a reusable handpiece assembly 15 in combination with all practitioner switches and displays enables the handpiece assembly 15 to be enclosed within a commercially available sterile, single-use, transparent and flexible sheath 109 as seen in FIG. 1B. The use of a sterile, transparent and flexible sheath 109 enables the tissue incision and retrieval system 10 of the present disclosure to be performed within the sterile field of an operating room without the requirement to sterilize the reusable handpiece assembly 15. By way of example and referring to FIG. 1B, distal end of sterile, single use sheath 109 includes an end plate 108 (e.g., circular cardboard disc) with hole 107 to allow passage of delivery cannula 22 and adhesive seal member at proximal end to assure disinfected but non-sterile handpiece assembly 15 remains fully enclosed in sterile sheath 109 and closed at its proximal end 110 during use in the sterile field of the operating room. The sterile sheath 109 that envelops handpiece assembly 15, as seen in FIG. 1B, is commercially available from Protek Medical Products, Inc. in Coralville, Iowa.

As seen in FIGS. 3, 4 and 7-7B, the motor-actuated first pivotable drive finger 185a extends upwardly such that it engages the drive block advancement ear 134 extending outwardly from drive assembly drive member 324. By way of example, translation nut 182a may be configured with a threaded portion to provide for secure attachment to first pivotable drive finger 185a. Upon insertion and full advancement of the single-use support housing 100 into the receiving cavity 166 of handpiece assembly 15, the first pivotable drive finger 185a abuts the surface of drive block advancement ear 134. Hereinafter, the position wherein the first pivotable drive finger 185a abuts the surface of drive block advancement ear 134 is referred to the “home” position of the first pivotable drive finger 185a.

In a preferred embodiment and referring to FIGS. 1, 1A, 1B and 3, rechargeable battery 183 provides the source of electrical power for circuit board 184 to enable all of the powering, display and practitioner control functions previously provided by an external control assembly in prior art apparatus and systems. Including practitioner actuation functions, visual cues and audible cues (e.g., the audible cue generated by speaker 200). The preferred embodiment eliminates the need for a costly external control assembly required in prior art apparatus and systems as well as eliminates the need for a connecting multi-lead cable, thereby enabling greater maneuverability of the handpiece assembly 15 by the practitioner during a surgical procedure. In addition, eliminating the need for a connecting multi-lead cable between the handpiece assembly 15 and an external control assembly, as seen in FIG. 1 of U.S. Pat. No. 6,471,659, enables and facilitates the complete enclosure of handpiece assembly 15 within a commercially available sterile, single-use, transparent and flexible sheath 109 as seen in FIG. 1B. the use of sterile sheath 109 allows procedures to be performed within the sterile field of an operating room without requiring the sterilization of the handpiece assembly 15.

Circuitry within the circuit board assembly 184 located within the handpiece assembly 15 continuously measures the voltage level applied to the resistively heated portion of cutting and pursing cable, i.e., that portion of the cutting pursing cable residing within and distal to the first and second electrically and thermally conductive eyelets 446 and 450, respectively, that supply constant current to the resistively heated portion of cutting and pursing cable as seen in FIG. 11. Momentarily referring to FIGS. 3, 11 and 17, the total electrical resistance, R_{leads} of all of the electrical leads 187, 189, 114, 116, 444 and 448 between the [a] circuit board assembly 184 and [b] first and second electrically and thermally conductive eyelets 446 and 450, respectively, is negligibly small relative to the linear electrical resistance of the resistively heated portion of cutting and pursing cable. As a result, the applied and measured voltage difference between first and second constant current source connector pins 51 and 53 accurately represents the voltage difference between first and second electrically and thermally conductive eyelets 446 and 450, respectively (i.e., the voltage level or voltage difference applied to the resistively heated portion of cutting and pursing cable). Ratiometrically combining the voltage level or voltage difference applied to first and second electrically and thermally conductive eyelets 446 and 450, respectively, as continuously measured within the circuit board assembly 184 with the continuously measured constant current level enables the continuous determination of the total electrical resistance of the resistively heated cutting and pursing cable, R_{cable}.

As seen in FIGS. 3 and 9, during the application of constant current to the first through sixth segments of the resistively heated portion of the cutting and pursing cable 89-94, circuitry within the circuit board assembly 184 continuously measures the total electrical resistance of the resistively heated portion of the cutting and pursing cable circuit. The electrical resistance of the resistively heated portion of the cutting and pursing cable circuit comprises [a] the electrical resistance of the first through sixth segments of the resistively heated portion of the cutting and pursing cable 89-94 and [b] the electrical contact resistances at the sliding interfaces between the resistively heated portion of the cutting pursing cable and the contacted surfaces of the first and second electrically and thermally conductive eyelets 446 and 450, respectively. When the continuously measured electrical resistance of the resistively heated portion of the cutting and pursing cable circuit decreases below a predetermined level (e.g., 2.0 ohms), then the circuitry within the circuit board assembly 184 determines that the pursing down of the distal ends of leaf members 82-86 and distal end multi-lumen flexible polymeric extrusion member 426 (see FIGS. 21 and 28) has been completed also known as the “Capture Complete” state. Once the “Capture Complete” state is reached, the applied constant current and applied constant voltage is suspended and the “Capture Complete” indicator light 52 is illuminated.

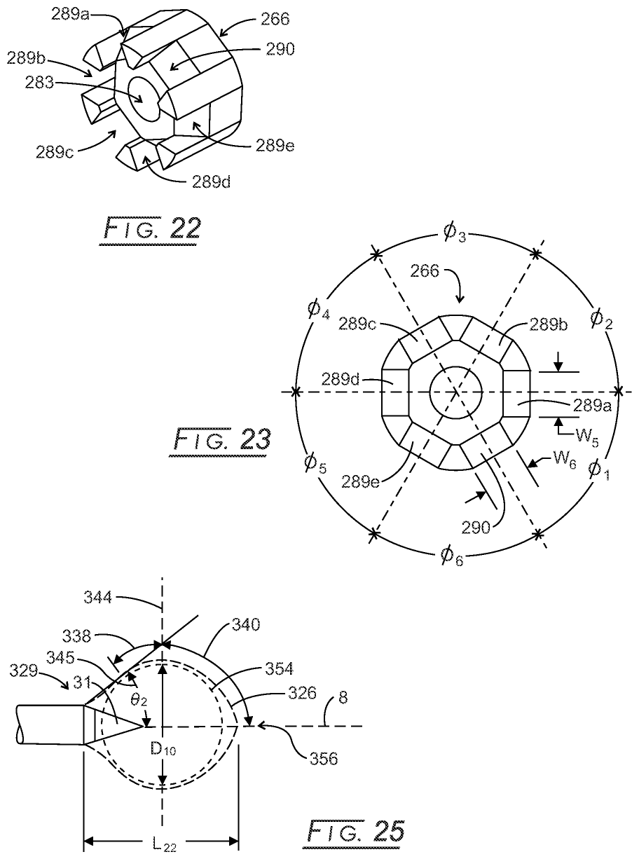

The single-use tissue incision and retrieval assembly 12 with single-use support housing 100 and delivery cannula 22 is illustrated in detail in connection with FIGS. 1A, 2, 4, 12, 21 and 22. Single-use support housing 100 is formed of two molded housing halves, which are joined together, additionally interconnected with the delivery cannula 22 and threaded locking 26. The embodiment of these figures shows delivery cannula distal end assembly 25 at distal tip of forward end 27 of delivery cannula 22 that incorporates tip component 266 (seen in FIG. 22), blade support 230 and blade 31. As seen in FIGS. 22 and 23, tip component 266 provides five deployment ramps 289a-289e for leaf members 82-86, respectively, and one ramp member 290 for the wider multi-lumen flexible polymeric extrusion assembly 426. The deployment ramps 289a through 289e and 290 affect the deployment angle, Θ₂ of deployment of the five leaf members 82-86 as well as the angle of deployment of the multi-lumen flexible polymeric extrusion member 426 as seen in FIGS. 21, 22 and 25. A surgically sharp cutting blade 31 is shown in these figures in the manner as described in connection with FIGS. 1A, 2 and 12.

Referring to FIGS. 2 and 4, a sectional view of the support housing 100 is revealed showing its formation from two single-use casings, an upper half 270 and a lower half 272 that are preferably joined together by the application of adhesive (e.g., cyanoacrylate) along the joint line 21 of single-use housing 100. Note that single-use casings 270 and 272 are securely attached to delivery cannula 22 at their forward portions by the application of adhesive (e.g., cyanoacrylate). Cannula 22 is seen to be a hollow tube. As seen in FIGS. 2 and 8 at the opposite sides of upper half of casing 270 and lower half of casing 272, first and second electrical contacts 120 and 122 are positioned near the distal end of single-use support housing 100. By way of example, upper half of casing 270 and lower half of casing 272 may be injection molded using an electrically insulative plastic such as widely available injection moldable polycarbonate resin materials.

Referring now to FIGS. 4, 8 and 20, extending from a proximal end face represented generally at 64 and defined by molded components of support housing 100 single-use casings 270 and 272, there is provided an elongate support tube 282. By way of example, support tube 282 is formed of stainless steel (e.g., stainless steel Type 304 tubing available from Micro Group, Inc., Medway, Massachusetts) and is anchored at the rearward side of proximal end face 64 by threaded rod 177 adhesively and/or mechanically locked into position on support tube 282. The support tube is secured at rearward side of proximal end face 64 by support tube tensioning nut 63 engaged with threaded rod 177 as seen in FIG. 4. Support tube 282 extends symmetrically along longitudinal axis 8 for engagement with cannula distal end assembly 25 at forward end 27 of delivery cannula 22.

Referring to FIGS. 4 and 17, first and second tensionable portions 118, 119 of the cutting and pursing cables are shown in a cross-sectional view of support housing 100 and multi-lumen flexible polymeric extrusion member 426. In the cross-sectional views seen in FIG. 4, the proximal ends of first and second tensionable portions of cutting and pursing cable 118, 119 are secured to an electrically insulative cable mounting hub 296 with first and second cable fastening machine screws 487, 488. A stabilizing pivoting drive ear 492 extends from the body of the cable mounting hub 296 that slides in first elongate stabilizing slot 298 and prevents unwanted rotation of freely sliding cable mounting hub 296 during transport, handling and use of tissue incision and retrieval assembly 12. By way of example, the cable mounting hub body and pivoting drive ear may be an injection molded, electrically insulative plastic using widely available injection moldable polycarbonate resin materials.

Referring now to FIGS. 3, 5, 5A and 5B, the cable mounting hub 296 and pivoting drive ear 492 are seen in greater detail. As seen in FIG. 5B, pivoting drive ear 492 can rotate around the axis of pivot pin 491 from an upright orientation (as seen in FIG. 5) to a deflected orientation (as seen in FIG. 5B). Compressible resilient member 499 (e.g., foam rubber) enables the momentary deflection of pivoting drive ear 492 during insertion of support housing 100 into receiving cavity 166 thereby enabling the cable mounting hub 296 to be advanced past the first pivoting drive finger 185a extending from first translation nut 182a on first motor-actuated drive tube drive member translation assembly.

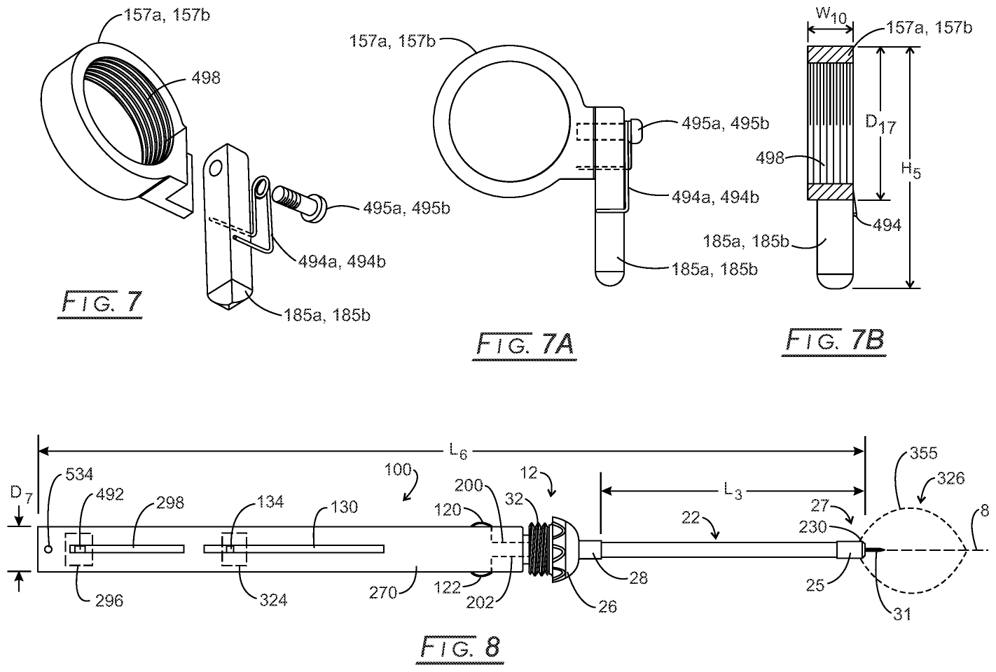

First and second drive nuts 182a and 182b are seen in FIG. 6, first and second drive nuts 182a and 182b are threaded at 496 to receive first and second pivoting drive finger supports 157a and 157b, respectively, having interior threads 498 as seen in FIGS. 7, 7A and 7B. First pivoting drive finger 185a is mounted on first pivoting drive finger support 157a and pivots about threaded pivot screw 495a in combination with toroidal spring 494a. Likewise, second pivoting drive finger 185b is mounted on second pivoting drive finger support 157b and pivots about threaded pivot screw 495b in combination with toroidal spring 494b. The first and second pivoting drive fingers 185a and 185b are momentarily deflectable as the single-use tissue incision and retrieval assembly is inserted into the handpiece assembly 15 and advanced beyond protruding drive block advancement ear 134 and pivoting drive ear 492 on cable mounting hub 296. Following passage beyond protruding drive block advancement ear 134 and pivoting drive ear 492, toroidal springs 494a and 494b return pivoting drive fingers 185a and 185b to their upright orientations to enable their intended function for advancement of drive assembly drive member 324 and cable mounting hub 296, respectively.

Referring now to FIGS. 3, 4, 21 and 26-28, the cable mounting hub 296 slides forwardly corresponding to the forward advancement of the five leaf members 82-86 as well as the multi-lumen flexible polymeric extrusion assembly 426 since the first and second tensionable portions of cutting and pursing cables 118 and 119, are secured at the cable mounting hub 296 and extend to the first through sixth segments of the resistively heated portion of the cutting and pursing cable 89-94. In this arrangement, the cable mounting hub 296 continues to advance forward as the leaf members and flexible polymeric extrusions advance forward until pivoting drive ear 492 that extends from cable mounting hub 296 abuts stationary second drive finger 185b that extends from second motor-actuated cable mounting hub translation assembly. Once pivoting drive ear 492 abuts the stationary second drive finger 185b, the continuously measured electrical current supplied to the first motor-actuated drive tube drive member 180a will immediately and measurably increase due to the tensile forces associated with start of the purse down of the distal ends of the five leaf members 82-86 and the multi-lumen flexible polymeric extrusion assembly 426. Upon the detection of a sudden increase in the measured electric current supplied to the first motor-actuated drive tube drive member 180a, the machine instructions within the circuit board assembly will initiate the application of a predetermined voltage to second motor 480b thereby advancing second drive finger 185b in a rearward direction 477 thereby increasing the purse down of the distal ends of the five leaf members 82-86 and the multi-lumen flexible polymeric extrusion assembly 426 at a predetermined rate to complete the closure of the tissue capture basket 326 as seen in FIG. 28.

In a preferred embodiment and referring to FIGS. 26-28, a compression spring 56 is positioned over the support tube 282 wherein the proximal end of the compression spring 56 abuts the distal face of the cable mounting hub 296 and the distal end of the compression spring 56 abuts a compression spring stop 505. The compression spring applies a force against the cable mounting hub 296 to maintain a predetermined minimum level of tension in the tensioning portions 118,119 of the cutting and pursing cable. Maintaining a minimum level of tension in the tensioning portions 118,119 of the cutting and pursing cable achieves acceptably low electrical contact resistance at the sliding electrical contact between the first and sixth resistively heated portions of the cutting and pursing cable 89 and 94 and electrically and thermally conductive eyelets 446 and 450, respectively, as seen in FIG. 11. By way of example, a small-diameter compression spring having a spring force constant, K of only about 0.1 pounds/inch of compression or other custom spring rate characteristics is available from D.R. Templeman, Plainville, Connecticut.

In order to more fully understand the apparatus structure and method of operation, the construction and functional regions of the cutting and pursing cable is now described in greater detail. Referring to FIGS. 4, 11 and 17, a first functional region of the cutting and pursing cable is that portion which is proximal to each electrically and thermally conductive eyelet located at the distal end of the multi-lumen flexible polymeric extrusion assembly 426. This first functional region is referred to as the tensionable portion of the cutting and pursing cable. The first tensionable portion 118 of the cutting and pursing cable extends from the cable mounting hub 296, continuing through lumen 438a in multi-lumen flexible polymeric extrusion 420, up to the first electrically and thermally conductive eyelet 446 located at the distal end of the multi-lumen flexible polymeric extrusion assembly 426. The second tensionable portion 119 of the cutting and pursing cable extends from the cable mounting hub 296 continuing through lumen 442a in multi-lumen flexible polymeric extrusion 420, up to the second electrically and thermally conductive eyelet 450 located at the distal end of the multi-lumen flexible polymeric extrusion assembly 426.

Referring now to FIGS. 4, 9, 11 and 17, a second functional region of the cutting and pursing cable is referred to as the resistively heated portion of the cutting and pursing cable. The second functional region of the cutting and pursing cable is that resistively heated portion of the cutting and pursing cable that begins at as well as distal to the electrically and thermally conductive eyelets located at the distal end of each multi-lumen flexible polymeric extrusion assembly. The first electrically and thermally conductive eyelet 446 includes a region of sliding electrical contact between the first electrically and thermally conductive eyelet 446 and the first tensionable portion of the cutting and pursing cable 118 as seen in FIG. 11. An electrical current flow path 399 is seen in FIG. 11 as it is conducted through first electrically and thermally conductive lead 444 to first electrically and thermally conductive eyelet 446. A first transition boundary 396 is at the locus of sliding contact between the cutting and pursing cable 33 and the first electrically and thermally conductive eyelet 446. This first transition boundary 396 represents the transition between the first segment of the resistively heated portion of the cutting and pursing cable 89 and the tensioning portion of the cutting and pursing cable 118. The current flow path 399 continues beyond first transition boundary 396 and into the first segment of the resistively heated portion of the cutting and pursing cable 89. The second electrically and thermally conductive eyelet 450 includes a region of sliding electrical contact between the second electrically and thermally conductive eyelet 450 and the second tensionable portion of the cutting and pursing cable 119 as seen in FIG. 11. An electrical current flow path 399 is seen in FIG. 11 as it is conducted through second electrically and thermally conductive lead 448 to second electrically and thermally conductive eyelet 450. A second transition boundary 406 is at the locus of sliding contact between the cutting and pursing cable 33 and the second electrically and thermally conductive eyelet 450. This second transition boundary 406 represents the transition between the sixth segment of the resistively heated portion of the cutting and pursing cable 94 and the tensioning portion of the cutting and pursing cable 119. The current flow path 399 continues beyond second transition boundary 406 and into the sixth segment of the resistively heated portion of the cutting and pursing cable 94.

As seen in FIGS. 9, 11 and 21, the electrical current flow path 399 continues sequentially through the entire continuous length of the resistively heated cutting and pursing cable, beginning through the first resistively heated portion of cutting and pursing cable 89 that extends from the first electrically and thermally conductive eyelet 446 at the distal end of multi-lumen flexible polymeric extrusion assembly 426 to a first eyelet 327a at the distal end of first leaf member 82. Next, the electrical current flow path 399 continues through a second resistively heated portion of cutting and pursing cable 90 that extends from a first eyelet 327a at the distal end of first leaf member 82 to a second eyelet 327b at the distal end of first leaf member 83. Next, the electrical current flow path 399 continues through a third resistively heated portion of cutting and pursing cable 91 that extends from second eyelet 327b at the distal end of second leaf member 83 to a third eyelet 327c at the distal end of third leaf member 84. Next, the electrical current flow path 399 continues through a fourth resistively heated portion of cutting and pursing cable 92 that extends from third eyelet 327c at the distal end of second leaf member 84 to a fourth eyelet 327d at the distal end of third leaf member 85. Next, the electrical current flow path 399 continues through a fifth resistively heated portion of cutting and pursing cable 93 that extends from fourth eyelet 327d at the distal end of second leaf member 85 to a fifth eyelet 327e at the distal end of third leaf member 86. Finally, the electrical current flow path 399 continues through a sixth resistively heated portion of cutting and pursing cable 94 that extends from fifth eyelet 327e at the distal end of second leaf member 86 to second electrically and thermally conductive eyelet 450 at the distal end of multi-lumen flexible polymeric extrusion assembly 426. This specified path of electrical current flow in the cutting and pursing cable is referred to as the resistively heated cutting and pursing cable circuit.

Referring now to FIGS. 9, 11 and 17, current flow 399 begins through first electrically and thermally conductive lead wire 444 to first electrically and thermally conductive eyelet 446 and continues through complete resistively heated cutting and pursing cable circuit to second electrically and thermally conductive eyelet 450 and continuing through second lead wire 448. First and second electrically and thermally conductive leads 444 and 448 continue proximally to first and second lead wires 114 and 116, respectively, that terminate at first and second electrical contacts 120 and 122, respectively, located on left and right sides, respectively, of single-use support housing 100 as seen FIG. 8.

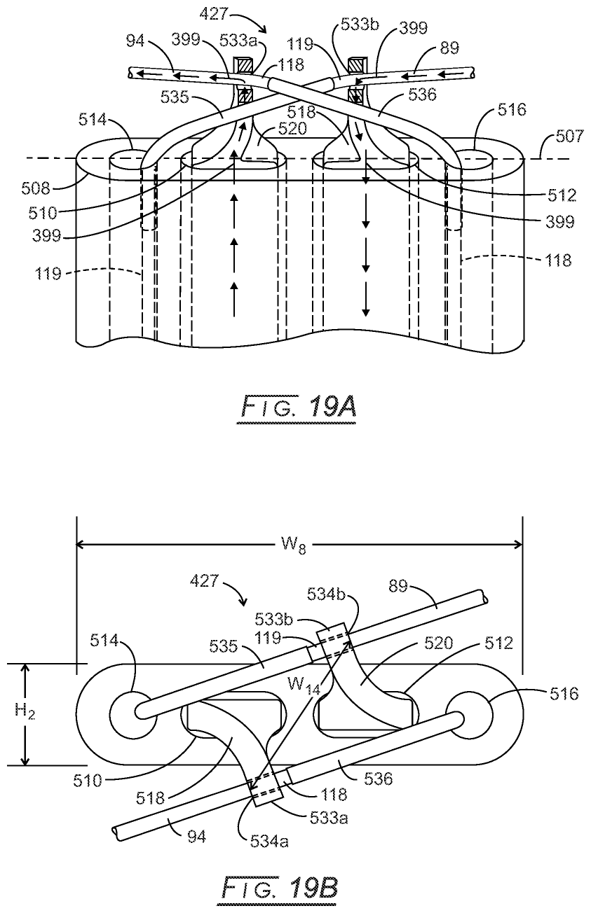

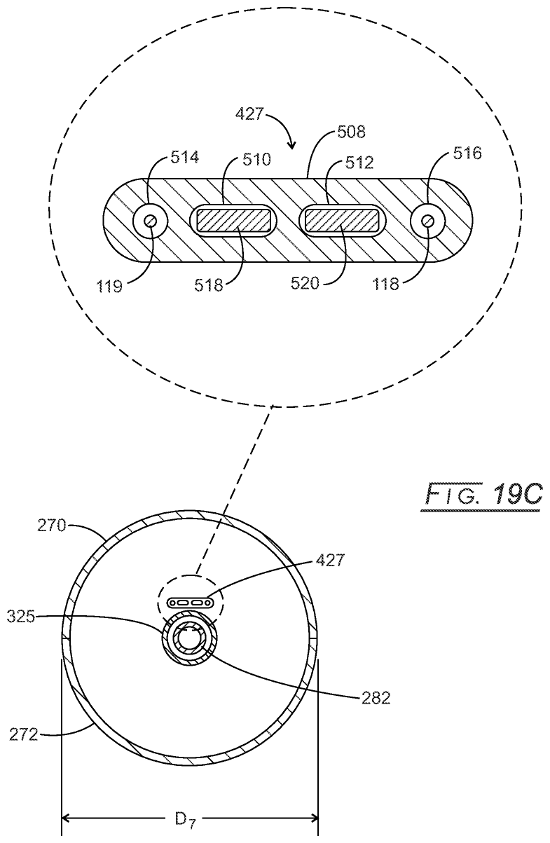

An alternative embodiment for a multi-lumen flexible polymeric extrusion assembly is the multi-lumen flexible polymeric extrusion assembly 427 incorporating two oval lumens and two round lumens is seen in FIGS. 18, 19A, 19B and 19C. Turning first to FIG. 18, and end-view of multi-lumen flexible polymeric extrusion member 508 reveals two oval lumens 510 and 512 in the central portion of multi-lumen flexible polymeric extrusion member 508 with round lumens 514 and 516 at the outer portions of multi-lumen flexible polymeric extrusion member 508 having specified dimensions as disclosed herein.

The oval lumens 510 and 512 within multi-lumen flexible polymeric extrusion member 508 enable the use flat electrically and thermally conductive lead wires 518, 520 having a through hole of diameter D5 that functions as an eyelet 533 as seen in FIG. 19. As seen in FIGS. 18 and 19A, flat electrically and thermally conductive lead wires 518, 520 advantageously impart greater stiffness (i.e., resistance to deflection) along the axis 507 of multi-lumen flexible polymeric extrusion assembly 427 thereby maintaining a minimum spacing between adjacent flat electrically and thermally conductive lead wires 518 and 520 as well as their respective electrically and thermally conductive eyelets 533a and 533b, respectively, in the presence of the tensile loads applied to electrically and thermally conductive eyelets 533a and 533b during the purse down of the tissue capture basket 326 (not shown). The preferred use of silver (99.5% purity or greater) for the forming of the first and second flat electrically and thermally conductive lead wires 518 and 520 provides a low electrical resistance path for the conduction of constant current to first through sixth segments of the resistively heated portion of the cutting and pursing cable 89-94. In addition, the use of silver (99.5% purity or greater) for the forming of the flat electrically and thermally conductive lead wires 518 and 520 provides a low thermal resistance path for the conduction of heat away from the electrically and thermally conductive eyelets 533a and 533b associated with the resistively heated portions 89 and 94 whose temperature is at least about 400° C. due to Joulean heating induced by the flow of constant current within the resistively heated portion of the cutting and pursing cable 33. In addition, the use of silver (99.5% purity or greater) is known to be biocompatible.

In the preferred embodiment of the multi-lumen flexible polymeric extrusion assembly 427 seen in FIGS. 19A and 19B, first tensioning portion of cutting and pursing cable 118 exiting lumen 516 extends past second electrically and thermally conductive eyelet 533b without making electrical contact by providing physical separation and/or electrical insulation between the first tensioning portion of cutting and pursing cable 118 and second electrically and thermally conductive eyelet 533b. The first tensioning portion of cutting and pursing cable 118 continues and passes through first electrically and thermally conductive eyelet 533a to become the first segment of the resistively heated portion of the cutting and pursing cable 89. Likewise, the second tensioning portion of cutting and pursing cable 119 exiting lumen 514 extends past first electrically and thermally conductive eyelet 533a without making electrical contact by providing physical separation and/or electrical insulation between the second tensioning portion of cutting and pursing cable 119 and first electrically and thermally conductive eyelet 533a. The second tensioning portion of cutting and pursing cable 119 continues and passes through second electrically and thermally conductive eyelets 533b to become the sixth segment of the resistively heated portion of the cutting and pursing cable 94. As seen in FIG. 19B, this cross-over arrangement of the first and sixth segments of the resistively heated portion of the cutting and pursing cable 89 and 94, respectively, advantageously enables the distance, W₁₄ to be minimized between [a] the point at which resistance heating begins 534a in first segment of the resistively heated portion of cutting and pursing cable 89 and [b] the point at which resistance heating begins 534b in sixth segment of the resistively heated portion of cutting and pursing cable 94. By minimizing the distance, W₁₄ the portion of tissue that is in confronting relationship to the advancing multi-lumen flexible polymeric extrusion assembly 427 while not being thermally cut by the first and sixth segments of the resistively heated portion of the cutting and pursing cable 89 and 94, respectively, is minimized. By minimizing the length of tissue not being thermally cut (i.e., by minimizing distance, W₁₄) the multi-lumen flexible polymeric extrusion assembly 427 is able to be advanced through unheated tissue through the process of mechanical severing of the tissue.

In a preferred embodiment and still referring to FIGS. 19A and 19B, the distal end of the first tensioning portion of cutting and pursing cable 118 is enclosed within an electrically insulative sleeve 536 that extends from the interior of lumen 516 to a position just proximal to the first electrically and thermally conductive eyelet 533a. Likewise, the distal end of the second tensioning portion of cutting and pursing cable 119 is enclosed within an electrically insulative sleeve 535 that extends from the interior of lumen 514 to a position just proximal to the first electrically and thermally conductive eyelet 533b. As seen in FIGS. 19A and 19B, the electrically insulative sleeve 536 prevents unwanted electrical contact between the first tensioning portion of cutting and pursing cable 118 and second electrically and thermally conductive eyelet 533b as well as prevents unwanted electrical contact between the first tensioning portion of cutting and pursing cable 118 and the second tensioning portion of cutting and pursing cable 119. Likewise, as seen in FIGS. 19A and 19B, the electrically insulative sleeve 535 prevents unwanted electrical contact between the second tensioning portion of cutting and pursing cable 119 and first electrically and thermally conductive eyelet 533a as well as prevents unwanted electrical contact between the second tensioning portion of cutting and pursing cable 119 and the first tensioning portion of cutting and pursing cable 118. By way of example, electrically insulative sleeves 535 and 536 may be small-diameter, thin-walled polyimide tubing having a wall thickness of 0.0005″ to 0.0010″ and commercially available from MicroLumen, Oldsmar, Florida.

Turning now to FIG. 19C, a sectional end-view of tissue incision and retrieval assembly taken through plane 19C-19C seen in FIG. 4. In this sectional end-view, multi-lumen flexible polymeric extrusion assembly 427 is seen in annular space between exterior surface of drive tube 325 and interior surfaces of upper half 270 and lower half 272 of single-use support housing 100. As seen in FIG. 19C, the multi-lumen flexible polymeric extrusion assembly 427 incorporates two round lumens 514 and 516 that serve as conduits for the unimpeded movement of first and second tensioning portions of cutting and pursing cable 119 and 118, respectively. Also, multi-lumen flexible polymeric extrusion assembly 427 incorporates two oval lumens 510 and 512 that serve as conduits for stationary first and second flat electrically and thermally conductive lead wires 518 and 520.

Hereinafter in the present disclosure, references to the multi-lumen flexible polymeric extrusion assembly may apply interchangeably to either [a] the multi-lumen flexible polymeric extrusion assembly 426 seen in FIG. 11 having four round lumens 434a, 438a, 440a and 442a as well as first and second electrically and thermally conductive eyelets 446 and 450, respectively or [b] the multi-lumen flexible polymeric extrusion assembly 427 seen in FIGS. 19A and 19B having two oval lumens 510 and 512 as well as two round lumens 514 and 516 in combination with first and second electrically and thermally conductive eyelets 533a and 533b, respectively.

As seen in FIGS. 3 and 8, first and second electrical contacts 120 and 122 located on the left and right sides, respectively, of single-use support housing 100 are in electrical communication with corresponding first and second electrical contact terminals 186 and 188, respectively, located on inner wall of handpiece assembly 15 when single-use support housing 100 is fully inserted into handpiece assembly 15. The proper orientation of single-use support housing 100 within handpiece assembly 15 during insertion into receiving cavity 166 is maintained by the confinement of guide pin 534 as it enters alignment key notch 167 and continues along third elongate slot 35 located within handpiece assembly 15. As seen in FIG. 3, electrical current flow path 399x continues from first connector pin 51 on constant current source 247 to first constant current connector 187a and along first lead 187 that is in electrical communication with first electrical contact terminal 186. Likewise, electrical current flow path 399z continues to connector pin 53 on constant current source 247 from second constant current connector 189a at proximal end of second lead 189 that is in electrical communication with second electrical contact terminal 186.

Referring now to FIGS. 9, 22 and 23, five leaf members 82-86 are progressively advanced through ramps 289a-289e, respectively, while simultaneously slightly wider multi-lumen flexible polymeric extrusion member 426 is progressively advanced through slightly wider ramp 290. As seen in FIG. 22, ramps 289a-289e and 290 are formed in tip component 266 located at distal end of delivery cannula 22. The progressive advancement of the five leaf members 82-86 and the multi-lumen flexible polymeric extrusion member 426 through their respective ramps causes the tissue capture basket 326 to expand into an increasingly larger circumscribing extent. By way of example, four stages of progressive advancement of leaf members 82-86 and multi-lumen flexible polymeric extrusion member 426 are illustrated in FIG. 9 wherein the lengths of the six segments of the resistively heated portion of cutting and pursing cable 89 through 94 are seen to increase in correspondence with the progressive advancement of the leaf members and multi-lumen flexible polymeric extrusion member. The four stages of advancement of the tissue capture basket 326 are designated by the letter suffix a, b, c or d appended to the number references for the leaf members 82-86, multi-lumen flexible polymeric extrusion member 426 as well as the first through sixth segments of the resistively heated portion of the cutting and pursing cable 89-94.

As seen in FIG. 9, the maximum diameter extent of the tissue capture basket 326 is designated D₁₁, D₁₂, D₁₃ and D₁₄ corresponding to the first (initial), second, third and fourth stages of the progressive advancement of the leaf members and multi-lumen flexible polymeric extrusion members. By way of further example in FIG. 9, the fourth stage of the progressive advancement of the leaf members 82-86 and the multi-lumen flexible polymeric extrusion member 426 results in the first through sixth resistively heated portions of cutting and pursing cables 89d-94d having a maximum diameter extent of D₁₄ corresponds to the maximum diameter, D₁₀ of the target tissue volume 354 seen in FIG. 25.

As described in the foregoing specification and seen in FIGS. 11 and 17, the cutting and pursing cable is partitioned into two discrete functional regions. A first functional region includes the tensioning portions 118 and 119 of the cutting and pursing cable wherein no electrical current flows. The tensioning portion of each cutting and pursing cable is proximal to that portion of the cutting and pursing cable that is in sliding electrical contact with the electrically and thermally conductive eyelets 446 and 450 that serve as electrodes for the conduction of constant current between the electrically and thermally conductive eyelets and the resistively heated portion of the cutting and pursing cables. A second functional region is the resistively heated portion of each cutting and pursing cable wherein constant current begins to flow within the resistively heated portion of the cutting and pursing cable at the locus of sliding electrical contact (seen at first and second transition boundaries 396 and 406 in FIG. 11) as well as distal to the electrically and thermally conductive eyelets 446 and 450. The confinement of constant current flow within only the resistively heated portion of the cutting and pursing cable that is in contact with and distal to the electrically and thermally conductive eyelets 446 and 450 corresponds to the confinement of constant current flow within only the resistively heated portion of the cutting and pursing cable that is in direct thermal contact with and dissipating heat into the tissue being incised. The confinement of constant current flow within only the resistively heated portion of the cutting and pursing cable that is in direct thermal contact with the tissue is essential for the controlled thermal incision of tissue by the resistively heated portion of the cutting and pursing cable without overheating and melting or fracturing the resistively heated portion of the cutting and pursing cable.

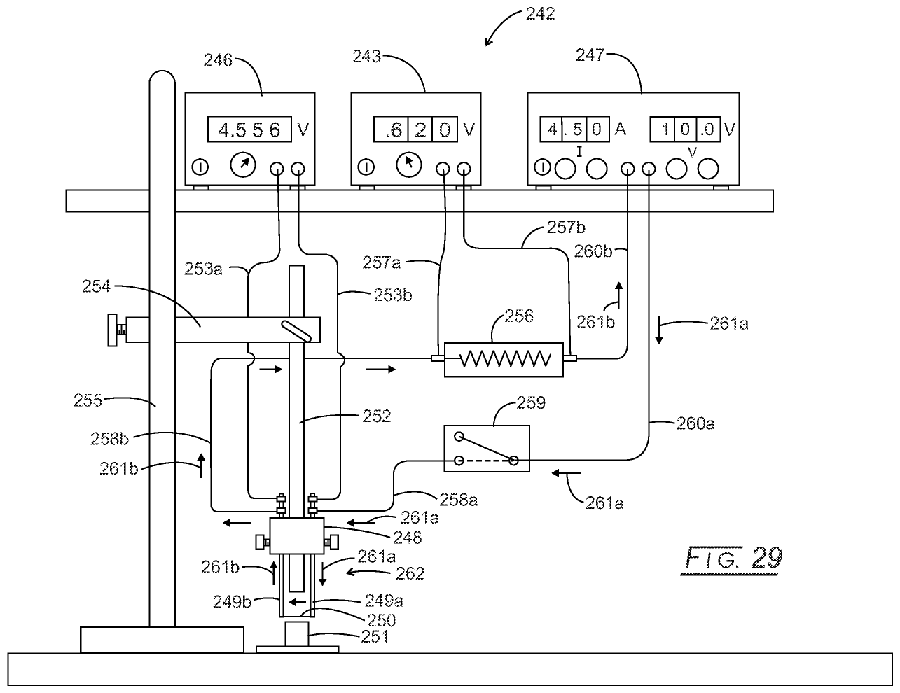

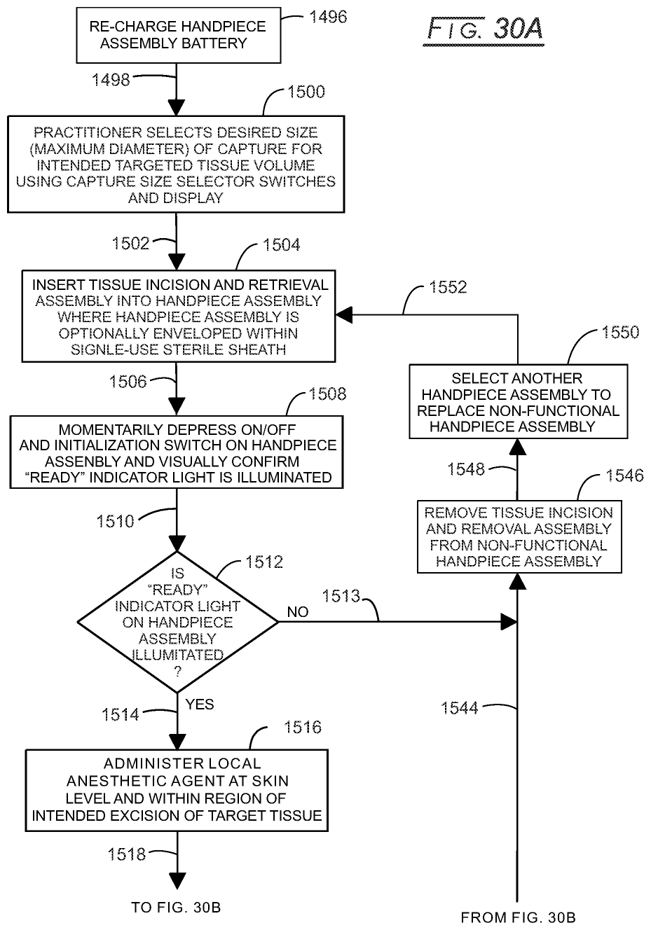

The essential requirement that constant current be confined to flow within only the resistively heated portion of the cutting and pursing cable that is in contact with and distal to the electrically and thermally conductive eyelets as well as the confinement of the flow of constant current within only the resistively heated portion of the cutting and pursing cable that is in direct thermal contact with tissue is more fully understood by examining the rate of power dissipation and corresponding temperature of the cutting cable that is required to incise tissue. In this regard and referring to FIG. 29, an experimental ex vivo tissue cutting apparatus 242 was designed to accurately measure the electrical resistance of a platinum wire 250 suspended between two silver tubular supports 249a and 249b while applying a known constant current level. A small-diameter solid platinum wire 250 was selected to simulate the first through sixth segments of the resistively heated portion of the cutting and pursing cable 89-94. Preferably, the cutting and pursing cable is fabricated in the form of a highly flexible cutting and pursing cable 33 having a multiplicity of small wires 34a-34g as seen in FIG. 9A. The diameter of each wire comprising cutting and pursing cable 33 may be in the range from about 0.0010 to 0.0015 inch with a preferred cable construction having seven wires and an overall diameter D₁₆ of preferably in the range from 0.039 to 0.045 inch as seen in FIG. 9A. The composition of the individual wires 34a-34g in cutting and pursing cable 33 is preferably a cobalt/chromium/tungsten/nickel alloy (commonly referred to as L605 alloy or Haynes 25 alloy) and is commercially available from Fort Wayne Metals, Inc. in Fort Wayne, Indiana. Alternatively, the cable may comprise multiple wires of stainless steel Type 304 or 316 and is commercially available from Jersey Wire & Cable, Inc. in Phillipsburg, New Jersey.

Based on the preferred range of diameters for the cutting and pursing cable, the two diameters of the substantially pure platinum wire used in the bench-top cutting of samples of ex vivo animal tissue 251 (e.g., steak tissue) using the test fixture schematic seen in FIG. 29 were either 0.003 inch or 0.005 inch. A platinum wire 250 wire of the selected diameter was inserted into the ends of two silver tubes 249a and 249b, each tube having an outside diameter of 0.040 inch and inside diameter of 0.020 inch and an overall length of about 2.5 inch. The two silver tubes 249a and 249b were separated by a distance of nominally 0.8 inch by an electrically insulative silver tube holder 248 as seen in FIG. 29. The platinum wire 250 of the selected wire diameter was soldered into the ends of the silver tubes using 60% tin/40% lead solder having a melting point of 188° C. The soldered joint assures that the contact resistances between the platinum wire 250 and the supporting silver tubes 249a and 249b are negligible. Also, the total resistance of the two silver tubes 249a and 249b supporting the platinum wire 250 only adds about 0.003 ohms of electrical resistance to the measurement of the platinum wire electrical resistance which represents only about 0.2% to 0.6% of the resistance of the platinum wire 250 during the heating of the platinum wire 250 by the application of a known constant current level. Vertically slideable assembly 262 comprises electrically insulative silver tube holder 248, silver tubes 249a, 249b and platinum wire 250 soldered across the distal ends of the silver tubes.

The two selected diameters of platinum wire 250 in the experimental ex vivo tissue cutting apparatus 242 seen in FIG. 29 were selected to closely approximate the range of diameters of the cable used in the cutting and capture assembly 329 as seen in FIG. 25. Accordingly, the heat flux levels and range of cutting wire temperatures measured using the experimental arrangement seen in FIG. 29 are equivalent to the heat flux levels and range of cutting wire temperatures that are attained by the flexible, multi-wire cutting and pursing cable used in the cutting and capture assembly 329 of the present disclosure.

The silver tubes 249a and 249b are supported in an electrically insulative silver tube holder 248 that maintains the length of the heatable platinum wire 250 during each cutting test on sample of ex vivo animal tissue 251 as seen in FIG. 29. As specified above, the length of the heatable platinum wire 250 during each cutting test was about 0.8 inch. The weight of the silver tube holder 248 provides the force required to advance the heated platinum wire 250 through the sample of ex vivo animal tissue 251 when the pre-selected level of sufficient constant current is applied since the silver tube holder 248 slides vertically and freely on guide rod 252 maintained in a vertical orientation by guide rod support 254 attached to ring stand 255 as seen in FIG. 29. A constant current source 247 (e.g., a commercially available regulated DC power supply) provided a source of constant current 261 at pre-selected constant current levels flowing in first constant current lead segment 260a, second constant current lead segment 260b, third constant current lead segment 258a and fourth constant current lead segment 258b as seen in FIG. 29. The actual level of the applied constant current is determined by measuring the voltage difference between the ends of a precision resistor that functions as current shunt 256. During the cutting tests on a sample of ex vivo animal tissue 251, the maximum power dissipated in the current shunt 256 is only about 6 watts and well within the 25-watt design limit for its use for current measurement.

The width of the sample of ex vivo animal tissue 251 during cutting with the heated platinum wire 250 is slightly less than the spacing between the inside boundary surfaces of the two silver tubes 249a and 249b so that the silver tubes 249a and 249b slide freely over the outside faces of the sample of ex vivo animal tissue 251 as seen in FIG. 29. A ruler with 1 mm divisions is placed in the field of view so that the rate of tissue cutting can be estimated using the video recorded images of the movement of the silver tube holder 248 and recording speed as the basis for calculating cutting speed.

Still referring to FIG. 29, the measurement procedure used to derive the platinum wire temperature during tissue cutting comprises the following steps:
1. Record and calibrate the measured voltage levels obtained with first digital voltmeter 243 and second digital voltmeter 246 using a nominal 1.5-volt primary battery and a nominal 9.0-volt primary battery.
2. Measure the fixed length of the platinum wire 250 suspended between the distal ends of the silver tubes 249a and 249b while mounted in the silver tube holder 248 using a digital caliper; measure the diameter of the platinum wire 250 using a digital micrometer.
3. Place a water-saturated paper towel segment (not shown) over the full length of the platinum wire 250 while it is suspended above the sample of ex vivo animal tissue 251 and apply a low level of constant current (e.g., 0.2 to 0.3 amps) using constant current source 247 while measuring the voltage difference across the current shunt 256 using first and second voltage sense leads 257a, 257b and the voltage difference across the two silver tubes 249a and 249b using third and fourth voltage sense leads 253a, 253b as shown schematically in FIG. 29. These two voltage measurements are next used to calculate the electrical resistance of the platinum wire at nominally 20° C. The water-saturated paper towel positioned over the length of silver wire 250 minimizes any unwanted heating of the platinum wire 250 substantially above 20° C. during the room-temperature resistance measurement of platinum wire 250 performed at a relatively low level of applied current.
4. Remove third and fourth constant current lead segments 258a and 258b, respectively, between the constant current source 247 and leading to the silver tubes 249a and 249b and briefly connect the constant current source 247 to a power resistor (not shown) of nominally 1.0 ohm and capable of dissipating 50 watts without significant temperature rise. This step is performed to adjust the constant current level of constant current source 247 for use in a particular test (e.g., 2.7 amps for one of the tests using 0.0030 inch diameter platinum wire) wherein the sample of ex vivo animal tissue 251 is cut with the heated platinum wire. After adjusting the constant current level of constant current source 247 to a pre-selected level, open the on/off switch 259 so that the on/off switch 259 is in the “off” position (i.e., open circuit condition).
5. Reconnect the third and fourth constant current lead segments 258a and 258b, respectively, between the constant current source 247 and the silver tubes 249a and 249b in preparation for the cutting of the sample of ex vivo animal tissue 251.
6. Start the video recording with the camera (not shown) with the video camera adjusted so that all of the meters, silver tube holder, silver tubes, sample of ex vivo animal tissue 251 and ruler (not shown) are in view during the recording process.
7. Close the position of the on/off switch 259 to the “on” position so that the pre-selected constant current 261 is flowing through the platinum wire 250.
8. Open the on/off switch position before the cutting wire advances through the full thickness of the sample of ex vivo animal tissue 251.
9. View video recording of cutting test to observe and document the digital meter readings and the rate of tissue cutting.

The electrical resistance of the nominal 0.003-inch diameter platinum wire 250 and the nominal 0.005-inch diameter platinum wire 250 used in cutting tests was determined at room temperature (nominally 20° C.) with auxiliary cooling of the platinum wire during the application of a constant test current of about 0.2 to 0.3 amps. The resistance of each platinum wire 250 was calculated using Ohms Law, wherein the electrical resistance, R (in unit of ohms) is equal to the voltage difference across an electrical conductor, V (in unit of volts) divided by the electrical current flowing through the conductor, I (in units of amperes). Likewise, during a tissue cutting test, a pre-selected constant current level 261 was applied to the platinum wire while it is in direct contact with the ex vivo animal tissue 251 as seen in FIG. 29. The constant current levels selected for the cutting of a sample of ex vivo animal tissue 251 provided the basis for selecting the range of resistive heating flux levels to be selected for subsequent in vivo cutting and capture of human tissue specimens using a stainless steel 316 cable or a cobalt/chromium/tungsten/nickel (e.g., L605) cable having a preferred diameter in the range from 0.0039 to 0.0045 inch.

The test procedure specified above was used to perform cutting tests performed on samples of ex vivo animal tissue 251 using a solid platinum wire 250 having a nominal diameter of 0.003 inch or 0.005 inch. The first step in the test protocol was to compare the voltage levels for two battery voltage sources, viz., a nominal 1.5-volt primary battery (not shown) and a nominal 9.0-volt primary battery (not shown). The measured voltage values using the second digital voltmeter 246 (used for measuring the voltage difference across the platinum wire support posts) and the first digital voltmeter 243 (used for measuring the voltage difference across the current shunt resistor) were within 0.05% and 0.07% for the nominal 1.5-volt and 9.0-volt battery sources, respectively. The confirmed close agreement between the two voltage measuring instruments, upon calibration in the voltage range to be used for the subsequent tests, assured the accuracy of the measurements of the resistance of the platinum wire 250 at 20° C. and subsequently the resistance of the platinum wire 250 at an elevated temperature during the period in which a constant current 261 is flowing in the platinum wire 250. In addition, since the electrical resistance of the platinum wire 250 under various applied constant current levels is calculated based on the ratio of the voltages measured using first digital voltmeter 243 and second digital voltmeter 246 during the cutting of each sample of ex vivo animal tissue 251, any bias in the closely agreeing voltage meters would effectively be cancelled out as a result of the ratiometric analysis.

Still referring to FIG. 29, once the electrical resistance of the platinum wire 250 was measured at nominally 20° C. and the constant current source 247 adjusted to deliver the pre-selected constant current level, then the platinum wire 250 was positioned so that the platinum wire rests on and is in direct contact with the surface of the sample of ex vivo animal tissue 251 while the silver tubes 249a and 249b are positioned adjacent to and just outside the vertical boundaries of the sample of ex vivo animal tissue 251 such that the downward movement of the silver tubes 249a and 249b are unimpeded by the sample of ex vivo animal tissue 251 and the heated platinum wire 250 advances through the sample of ex vivo animal tissue 251 at a rate impeded only by the rate of cutting of the sample of ex vivo animal tissue 251 by the heated platinum wire 250. Each test for a pre-selected constant current level was initiated by closing the on/off switch 259 to immediately commence the flow of constant current 261 through the platinum wire 250 as it begins to thermally cut through the sample of ex vivo animal tissue 251. In this regard, the cutting of tissue is properly defined as the thermal cutting of tissue. A heated wire operating a an insufficient constant current and that was incapable of cutting the sample of ex vivo animal tissue 251 in these bench-top tests was demonstrated to be operating below the threshold of thermal cutting of tissue. In tests wherein the level of applied current and associated heat flux was sufficiently high, the heated cutting wire achieved a heat flux and associated operating temperature that was capable of inducing the vaporization of liquid filled cellular components within the tissue, thereby causing the fracturing of cell membranes and effecting the incision or parting of the tissue.

The rate of cutting by the heated platinum wire 250 through the sample of ex vivo animal tissue 251 was determined primarily by the pre-selected constant current level. The higher the constant current level, the greater the heating rate generated within the platinum wire 250 and the greater the heat flux dissipated at the surface of the platinum wire 250 and, correspondingly, the higher rate of cutting of the sample of ex vivo animal tissue 251. Correspondingly, the higher the constant current level, the higher the temperature of the platinum wire 250 during the thermal cutting through the sample of ex vivo animal tissue 251.

For the case of the nominal 0.005-inch diameter platinum wire, an acceptably fast cutting rate through the ex vivo animal tissue 251 was achieved at a constant current level of 4.859 amps (see Test No. 1 in Table 1). An even faster rate of cutting rate through the ex vivo animal tissue 251 was achieved at a constant current level of 5.335 amps but significantly exceeded the actual cutting rate intended for the deployment of the cutting and capture assembly 329 through in vivo tissue tests in actual incision and capture of en bloc tissue samples within the body of human patients. For the case of an actual applied constant current level of 4.859 amps, the measured cutting rate was 2.3 mm/second. The measured cutting rate of 2.3 mm/second is within the preferred range of 2.0 to 3.0 mm/second for of advancement of the cutting and capture assembly 329 being driven by the first motor actuated drive tube drive member translation assembly 180a as seen in FIG. 3. These test results suggest that a nominal constant current level of 4.9 amps for a 0.005-inch diameter platinum heating wire provides a comparable rate of cutting as the preferred rate of advancement of the cutting and capture assembly 329 comprising a multi-lumen flexible polymeric extrusion assembly 426 and five leaf members 82-86 supporting the first through sixth segments of the resistively heated portion of the cutting and pursing cable 89-94 as described above with regard to FIG. 9.

For the case of the nominal 0.003-inch diameter platinum wire 250, an acceptably fast cutting rate through the sample of ex vivo animal tissue 251 was achieved at a constant current level of 2.709 amps as seen in Test Nos. 5 and 6 of

Table 1. For the case of an applied constant current level of 2.709 amps, the measured cutting rate was 2.8 to 2.9 mm/sec, which is slightly greater the intended rate of advancement of the cutting and capture assembly 329 and may enable the completion of the incision and capture of an en bloc tissue specimen within a patient during a shorter period of power application.

The calculated resistances of the heatable platinum wire at 20° C. and during the period of heating using an applied constant that provides an adequate cutting rate (e.g., 2.7 amps for the nominal 0.003-inch diameter platinum wire) were used in conjunction with the known temperature coefficient of resistance of platinum (viz., 3927 ppm/C) to calculate the temperature of the heated platinum wire during the application of a known constant current level. The video recording of the voltage values displayed by the first digital voltmeter 243 and second digital voltmeter 247 during the brief period (e.g., 2 to 3 seconds) that the platinum wire 250 traverses the sample of ex vivo animal tissue 251 was used to determine the average of the voltage differences between the two silver tubes 249a and 249b measured by the second digital voltmeter 246 and, hence, across the platinum wire 250 during the application of a constant current 261.

The electrical resistance, RT of a conductor (in Ohms) at an elevated temperature, T (in degrees Celsius) is given by the relationship:

$${R}_{T} = {R}_{0} ( 1 + a ( T - {T}_{0} ) ) \qquad \{ Equation 1 \}$$

where R₀ is the measured electrical resistance of the conductor at a known temperature, T₀ (in degrees Celsius) and a is the known temperature coefficient of electrical resistance of the conductor (in ppm per degree Celsius). For the case of a pure platinum conductor, the term a is equal to 3927 ppm/C or 0.003927 per degree C. Rearranging the terms of Equation 1 to express the unknown temperature, T as a function of the known and measured quantities gives the relationship:

$$T = ( ( {R}_{T} / {R}_{0} ) - 1 ) / a + {T}_{0} \qquad \{ Equation 2 \}$$

The above Equation 2 was used to calculate the temperature of the platinum heating wire 250 during the period in which a constant current 261 is applied to heat the platinum wire 250 and enable the cutting of sample of ex vivo animal tissue. The average of the calculated temperatures of the platinum wires 250 having nominal diameters of 0.003 inch and 0.005 inch for various levels of applied constant current are presented in Table 1.

As seen in Table 1 for Test No. 3, based on a heated 0.0048-inch diameter platinum wire 250 while cutting a sample of ex vivo animal tissue that is initially at room temperature (i.e., nominally 20° C.) and achieving a cutting rate that is comparable to the cutting rate using the motor-driven tissue cutting and capture assembly 329, the average calculated temperature of the platinum wire 250 is 373° C. By comparison, as seen in Table 1 for Test Nos. 5 and 6 based on a heated 0.0029-inch diameter platinum wire 250 while cutting a sample of ex vivo animal tissue 251 that is initially at room temperature (i.e., nominally 20° C.) and achieving a cutting rate that is comparable to the cutting rate using the motor-driven tissue cutting and capture assembly 329, the average calculated temperatures of the platinum wire temperature ranges from 358° C. to 363° C.

TABLE 1
Measured Heat Flux, Platinum Wire Temperature
and Rate of Cutting of Ex Vivo Animal Tissue

| | | | Average | Average | Cutting |
|---|---|---|---|---|---|
| | Platinum | Constant | Heat Flux | Platinum | Rate in |
| | Wire | Current | Dissipated | Wire | Animal |
| Test | Diameter | Level | from Wire | Temperature | Tissue |
| Number | (in) | (amps) | (watts/cm²) | (° C.) | (mm/sec) |
| 1 | 0.0048 | 3.932 | 86 | 298 | 1.0 |
| 2 | 0.0048 | 4.368 | 109 | 312 | 1.6 |
| 3 | 0.0048 | 4.859 | 150 | 373 | 2.3 |
| 4 | 0.0048 | 5.335 | 191 | 408 | 3.0 |
| 5 | 0.0029 | 2.709 | 220 | 363 | 2.8 |
| 6 | 0.0029 | 2.709 | 218 | 358 | 2.9 |
| 7 | 0.0029 | 3.010 | 284 | 393 | 5.4 |

Based on the results of the cutting tests in samples of ex vivo animal tissue 251 using the ex vivo animal tissue cutting apparatus 242 seen in FIG. 29, the heat flux dissipated from the cutting and pursing wire employed in the tissue incision and retrieval assembly 12 of the present disclosure is at least 150 watts/cm² and preferably 220 watts/cm². The temperature of the constant-current heated resistively heated portion of the cutting and pursing cable employed in the tissue incision and retrieval assembly 12 of the present disclosure during the cutting of tissue is preferably in the range from about 350° C. to 400° C.

The cutting tests in samples of ex vivo animal tissue 251 using the ex vivo animal tissue cutting apparatus 242, as seen in FIG. 29, determined that the thermal cutting of tissue at a preferred cutting rate ranging from 2.3 to 2.7 mm/second with small-diameter platinum wires ranging in diameter from 0.0030 to 0.0050 inch and heated to about 400° C. requires a wire or cable heat flux (i.e., heat generated per unit surface area of the cutting wire or cable) of at least 150 watts/cm² and preferably 220 watts/cm². As described above, the preferred heat flux required rate of thermal cutting of ex vivo animal tissue was determined using a solid platinum wire so that its measured resistance during thermal cutting could be used to determine the temperature of the platinum wire (based on the well-known principles of platinum wire resistance thermometry) during the preferred rate of the thermal cutting of ex vivo animal tissue.

Based on the established preferred heat flux from the multi-wire cutting cable (viz., at least 150 watts/cm² and preferably 220 watts/cm²), the level of constant current that needs to conducted through the first through sixth segments of the resistively heated portion of the cutting and pursing cable 89-94 can be derived using the Joule-Lenz Law (also known as Joule's First Law combined with Ohm's Law). According to the Joule-Lenz Law, the power or the heating, P (also known as Joulean heating) can be expressed as the product of the square of the current, I and the resistance of the conductor, R as seen below in Equation 3:

$$P = {I}^{2} \times R \qquad \{ Equation 3 \}$$

The minimum and preferred heat fluxes (i.e., heating power, P dissipated per unit surface area, A of the cutting wire or cable) of 150 watts/cm² and 220 watts/cm², respectively, can be converted into the unit of heating power per unit length, L of the first through sixth segments of the resistively heated portion of the cutting and pursing cable 89-94 by dividing the preferred heat flux by the circumference, C_{cable} of the assumed-round first through sixth segments of the resistively heated portion of the cutting and pursing cable 89-94. Recall that the surface area, A of a length, L of a round wire or cable having a diameter, D_{cable} is given by the equation:

$${A}_{cable} = {C}_{cable} \times L = 3.1 \; 416 \times {D}_{cable} \times L \qquad \{ Equation 4 \}$$

The cross-section of an individual cutting and pursing cable 33 within the tissue incision and retrieval assembly 12, is seen in FIG. 9A. The complete length of each individual cutting and pursing cable 33 comprises two functional regions, viz., the tensional portions of the cutting and pursing cable and the resistively heated portion of the cutting and pursing cable. As described above, the first and second tensionable portions of cutting and pursing cables 118 and 119, combine with the first through sixth segments of the resistively heated portion of the cutting and pursing cable 89-94, respectively, to form the complete length of the cutting and pursing cable. As seen in cross-sectional view FIG. 9A, cutting and pursing cable 33 comprises seven individual round wires 34a-34g of equal diameter, D₁₅ in which central wire 34a is symmetrically surrounded by six wires 34b-34g to form a cable having an effective diameter of D₁₆ as seen in FIG. 9A. Each of the seven wires 34a-34g is manufactured using a biocompatible, high-strength alloy in full-hard temper such as austenitic stainless steel 316 or the cobalt/chromium/tungsten/nickel alloy L605.

By way of example, a preferred construction for cutting and pursing cable 33 is manufactured by Fort Wayne Metals, Inc. (Fort Wayne, Indiana) and comprises seven individual cobalt/chromium/tungsten/nickel alloy L605 wires 34a-34g in full-hard temper, each wire 34 having a diameter, D₁₅ of 0.0013 inch to produce an overall nominal diameter, D₁₆ for cutting and pursing cable 33 of 0.0039 inch (0.00991 cm). The electrical resistance of this second preferred construction for cutting and pursing cable 33 was determined by measuring a known length of cable (e.g., 60 cm) at a room temperature of about 20° C. using a digital ohmmeter. The cable resistance per centimeter length was determined by dividing the measured resistance (in units of ohms) by the length of cable included in the resistance measurement. Using the above resistance measurement method, the resistance per unit length, R/L at 20° C. for the second preferred construction of cutting and pursing cable 33 was determined to be 1.63 ohms/cm. Based on measurements performed within the testing laboratory of the cable manufacturer, Fort Wayne Metals, the cable break strength at 20° C. for the second preferred cable construction was determined to be 3.18 pounds (force).

Referring now to Equation 4, the peripheral surface area of a unit length of cutting and pursing cable 33 of one centimeter is equal to the product of 3.1416 and the diameter, D₁₆ of cutting and pursing cable 33. For the case of a preferred cable design having a nominal cable diameter of 0.00991 cm, the surface area of cutting and pursing cable 33 per unit (centimeter) length is 3.1416×0.00991 cm×1.0 cm or 0.0311 cm². Based on the established minimum heat flux of 150 watts/cm² and preferred heat flux of 220 watts/cm² emanating from the surface of the multi-wire cutting cable, the amount of resistive heating power (i.e., Joulean heating) required per centimeter of cable length, P/L to generate the minimum heat flux of 150 watts/cm² and preferred heat flux of 220 watts/cm² can be derived by the product of the required minimum and preferred heat fluxes (viz., 150 and 220 watts/cm², respectively) and the surface area of a unit cable length of one centimeter (0.0331 cm²), the products computed to be 4.97 and 7.28 watts per centimeter length, respectively.

As described above and seen in FIG. 9, the minimum and preferred heat fluxes of 150 and 220 watts/cm², respectively, were experimentally determined based on the severing of ex vivo animal tissue with resistively heated platinum wires having diameters of 0.0030 and 0.0050 inches. It is hypothesized that the tissue cutting mechanism is due to the fracturing the cellular structures as a result of the vaporization of the contained liquid within each cell. Based on the minimum and preferred heat fluxes of 150 and 220 watts/cm², respectively, and the dimensions as well as the electrical resistance of the first through sixth segments of the resistively heated portion of the cutting and pursing cable 89-94 at 400° C., the actual amount of resistively generated power (i.e., power generated as a result of electrical current flowing through the inherent electrical resistance of the cable) that is dissipated per unit length of the resistively heated portion of cutting and pursing cables 89-94 is 7.28 watts/cm, respectively, for a cable diameter of 0.0039 inches.

The power dissipation rates per unit length for cutting and pursing cable 33 having a preferred diameter of 0.0039 inch were derived above based on measurements of the power, cutting wire temperature and heat fluxes required for the cutting of ex vivo animal tissue at a known cutting rates. These derived power dissipation rates per unit length for cutting and pursing cable 33 are next compared with the theoretical power dissipation rates per unit length of resistively heated portion of cutting and pursing cable derived based on the assumption of “thermal cutting” of tissue. As used herein, the term “thermal cutting” refers to a cutting mechanism wherein the incision of tissue is the result of the vaporization of liquid contained in the cells of tissue and consequent mechanical fracturing of the cell walls as a result of the volumetrically expanding vapor phase of liquid being vaporized. It is well known that cells in human or animal tissue contain about 70% water (see Cooper, G., The Cell: A Molecular Approach. 2000; Sinaeur Associates, Inc., Sunderland, Massachusetts). Assuming a preferred cutting and pursing cable diameter, D_{cable} of 0.0099 cm, a cutting rate of 2.3 mm/second and a cut duration, tout of 1.0 seconds during which the resistively heated portion of the cutting and pursing cable of length equal to 1.0 centimeter is advanced through tissue, the volume of tissue traversed by the resistively heated portion of cutting and pursing cable is the product of the width of the incision, W_{incision}×length of the cable, L_{cable}×length of the incision, L_{cut} wherein the width of the incision, W_{incision} 20% greater (or a factor 1.2 greater) than the resistively heated portion of cutting and pursing cable diameter, D_{cable} due to the combination of conduction and radiation heat transfer into the tissue on either side of the resistively heated portion of cutting and pursing cable. In this example, the volume of tissue thermally cut is 0.0099 cm×1.2×0.23 cm×1.0 cm or 0.0019 cm³. The corresponding mass of the tissue is the product of the total volume of tissue traversed by the resistively heated portion of cutting and pursing cable and the density of water (viz., 1.0 gram/cm³) or 0.0019 grams.

In adult women, the water-content represents between 50% and 70% of the mass of normal breast tissues. In this regard, see Conceicao, A. L C., et. al., The Influence of Hydration on the Architectural Rearrangement of Normal and Neoplastic Human Breast Tissues. Heliyon 2015; 5:1-13. The water-content, WC of the mass of tissue in the present example is assumed to be 70% to assure that the amount of energy transferred to the tissue during thermal cutting of tissue is sufficient to sustain the vaporization of cellular water that is required for the thermal cutting of tissue. Assuming water, having a density, d_{water} equal to 1.0 gram/cubic centimeter, comprises about 70% of the mass of tissue, then the mass, m_{water} of water vaporized for the resistively heated portion of cutting and pursing cable having length, L_{cable} of 1.0 cm is given by the equation:

$${m}_{w a t e r} = {d}_{w a t e r} \times ( {D}_{cable} \times 1.2 ) \times {L}_{cable} \times {L}_{cut} \times 70 \% \qquad \{ Equation 5 \}$$

By way of example, for the case of a preferred design for the cutting and pursing cable 33 seen in FIG. 9A, the diameter, D_{cable} of the cutting and pursing cable 33 is 0.0039 inch (0.00990 cm). Based on Equation 5, the mass, m_{water} of water vaporized for a cut width, W_{incision} Of D_{cable}×1.20 and cut length, L_{cut} of 0.23 cm is 0.0019 grams. The amount of thermal energy, Q_{cut} required to vaporize this mass of water for each cut length of 1.0 cm can be quantified by the addition of the sensible heat, Q_{sensible} (i.e., the heat required to heat water from an initial core body temperature of 37° C. to the nominal boiling point of water of 100° C. at atmospheric pressure of 760 mm Hg) and the latent heat of vaporization, LH_{water} of water, Qₗₐₜₑₙₜ as specified in the following equations:

$${Q}_{cut} = {Q}_{s ensible} + {Q}_{latent} \qquad \{ Equation 6 \}$$

$${Q}_{s ensible} = {m}_{w a t e r} \times {C}_{w a t e r} \times Δ T \qquad \{ Equation 7 \}$$

$${Q}_{latent} = {m}_{w a t e r} \times {LH}_{w a t e r} \qquad \{ Equation 8 \}$$

where C_{water} is the well-known specific heat of water (viz., 1 calorie/gram-C), ΔT is the temperature rise of cellular water at normal body temperature of 37° C. to the boiling point of water of 100° C. and LH_{water} is the well-known latent heat of vaporization of water of 540 calories/gram corresponding to the energy required to convert the liquid phase of water at 100° C. to the vapor phase of water at 100° C. Substituting these known values into Equations 6 through 8 corresponding to the assumed thermal cutting of tissue by vaporization of cellular water yields the following amounts of thermal energy per unit length of cut of 1.0 cm for the case of a cutting and pursing cable 33 having a diameter of 0.0039 inch:

$${Q}_{sensible} = 0.0 \; 019 grams \times 1. calorie / gram - C . ° \times ( 100 ° C . - 37 ° C . ) = 0.119 calories$$

$${Q}_{latent} = 0.0019 grams \times 540 calories / gram = 1.026 calories$$

$${Q}_{cut} = 0.119 + 1.026 = 1.1 \; 45 calories$$

Referring to FIGS. 3, 9, 11 and 19A, a more generalized form of Equation 6 can be expressed in terms of the diameter, D_{cable} of the resistively heated portion of cutting and pursing cable and the cutting rate, R_{cut} for a unit length of the resistively heated portion of cutting and pursing cable of 1.0 centimeter. The cutting rate, Rout corresponds to rate of advancement of the multi-lumen flexible polymeric extrusion assembly 426 or 427 and leaf members 82-86, a rate of advancement determined by the pre-selected voltage applied to motor 480a within the associated first motor-actuated drive tube drive member translation assembly 180a. The amount of energy required for the thermal cutting of tissue by the advancement of a the resistively heated portion of cutting and pursing cable having a diameter, D_{cable}, length of cut, L_{cut} and water content fraction, WC can be specified as seen in the following equations by combining Equations 5, 6, 7 and 8:

$${Q}_{cut} = {d}_{w a t e r} \times ( {D}_{cable} \times 1.2 ) \times {L}_{cable} \times {L}_{cut} \times WC \times ( {C}_{w a t e r} \times Δ T + L {H}_{w a t e r} ) \qquad \{ Equation 6 A \}$$

wherein the multiplying factor 1.2 refers to the increased effective width of the path of thermal cutting of tissue due to heat that is conducted and radiated into the tissue beyond the boundary diameter, D_{cable} of the resistively heated portion of cutting and pursing cable as it advances through tissue.

The length of the tissue being thermally cut, L_{cut} can be expressed as the product of the cutting rate, R_{cut} and the duration of cut, t_{cut} as follows:

$${L}_{cut} = {R}_{cut} \times {t}_{cut} \qquad \{ Equation 6 B \}$$

Substituting Equation 6B into Equation 6A yields the following equation:

$${Q}_{cut} = {d}_{w a t e r} \times ( {D}_{cable} \times 1.2 ) \times {L}_{cable} \times {R}_{cut} \times {t}_{cut} \times WC \times ( {C}_{w a t e r} \times Δ T + L {H}_{w a t e r} ) \qquad \{ Equation 6 C \}$$

wherein the density of cellular water, d_{water} has the value 1.0 gram/cm³, D_{cable} is in units of centimeters, R_{cut} is in units of centimeters/second, tout is in units of seconds, WC is a unitless fractional value corresponding to water content, C_{water} is in units of calories/gram-° C., LH_{water} is in units of calories/gram, Q_{cut} is in units of calories and the length of the resistively heated portion of cutting and pursing cable that thermally cuts through tissue is fixed value of 1.0 centimeter for the purpose of calculations.

The amount of energy, Q_{cut} delivered to a length of tissue being cut, L_{cut} by the resistively heated portion of cutting and pursing cable (in units of calories) as seen in Equation 6C can be expressed as a rate of energy application per unit time, Q_{cut}/t_{cut} (in units of calories/second) by dividing both sides of Equation 6C by the duration of cut, tout, and assigning a value of 1.0 second to the duration of cut, tout yielding the following equation:

$${Q}_{cut} / sec = {d}_{w a t e r} \times ( {D}_{cable} \times 1.2 ) \times {L}_{cable} \times {R}_{cut} \times WC \times ( {C}_{w a t e r} \times Δ T + L {H}_{w a t e r} ) \qquad \{ Equation 6 D \}$$

The amount of thermal energy, Q_{cut} required for the thermal cutting of tissue for a duration of cut, tout having the value 1.0 second as seen in Equation 6D can next be converted into the amount of power required, Pout for the thermal cutting of tissue by converting the energy unit calories into Joules. Since 1.0 calorie is equivalent to 4.186 Joules and 1.0 Joule/second is equivalent to 1.0 watt, then Equation 6D can be expressed as follows:

$${P}_{cut} = 4.1 \; 86 \times {d}_{w a t e r} \times ( {D}_{cable} \times 1.2 ) \times {L}_{cable} \times {R}_{cut} \times WC \times ( {C}_{w a t e r} \times Δ T + L {H}_{w a t e r} ) \qquad \{ Equation 6 E \}$$

By way of example, the well-known values for the density of water (viz., 1.0 gram/cm³), an length of the resistively heated portion of cutting and pursing cable of 1.0 centimeter for the purpose of calculations, the specific heat of water, C_{water} (Viz., 1.0 calorie/gram-° C.), the latent heat of vaporization of water, LH_{water} (viz., 540 calories/gram), the amount of increase in tissue temperature, ΔT to elevate cellular water from a normal body temperature of 37° C. to the boiling point of water of 100° C. (viz., 63° C.) and the published water content of breast tissue (viz., 70%) can be substituted into Equation 6E. In addition, also substituting the values of the diameter of the resistively heated portion of cutting and pursing cable, D_{cable} (viz., 0.0099 centimeters) and rate of cutting, R_{cut} (viz., 0.23 centimeters/second) used by the applicant in laboratory testing of a prototype tissue incision and retrieval system as seen in FIG. 1 yields a value for P_{cut} specified in Equation 6E of 4.83 watts.

In the above example, the calculated amount of power or resistance heating (viz., 4.83 watts) represents the minimum amount of power that needs to applied to the six segments of the resistively heated portion of the cutting and pursing cable 89-94 (as seen in FIG. 9) a cable diameter, D_{cable} of 0.0099 cm, for each 1.0 cm length of cable and for a rate of advancement of the resistively heated portions of the cutting and pursing cable of 0.23 cm/sec.

The amount of resistance heating, also known as Joulean heating, in the resistively heated portions of the cutting and pursing cable can also be expressed in terms of the cable electrical resistance, R_{cable} and the minimum level of substantially constant current, I_{cut} conducted through the resistively heated portion of the cutting and pursing cable as specified in Equation 3 above. Substituting Equation 3 into Equation 6E yields:

$${P}_{cut} ( watts ) = {( {I}_{cut} )}^{2} \times {R}_{cable} \qquad \{ Equation 6 F \}$$

$${( {I}_{cut} )}^{2} \times {R}_{cable} = 4.1 \; 86 \times {d}_{w a t e r} \times ( {D}_{cable} \times 1.2 ) \times {L}_{cable} \times {R}_{cut} \times WC \times ( {C}_{w a t e r} \times Δ T + L {H}_{w a t e r} ) \qquad \{ Equation 6 G \}$$

$${I}_{cut} = {[ 1 / {R}_{cable} \times 4.186 \times {d}_{w a t e r} \times ( {D}_{cable} \times 1.2 ) \times {L}_{cable} \times {R}_{cut} \times WC \times ( {C}_{w a t e r} \times Δ T + L {H}_{w a t e r} ) ]}^{0.5} \qquad \{ Equation 6 H \}$$

Expressing Equation 6G in terms of the minimum level of substantially constant current, I_{cut} required for the thermal cutting of tissue provides an equation for the calculation of the minimum level of substantially constant current required, I_{cut} to achieve the thermal cutting of tissue for [a] a selected cable material having a resistance per 1.0 cm length of cable, R_{cable}, [b] a diameter of the resistively heated portion of the cutting and pursing cable, D_{cable}, [c] a selected tissue cutting rate, R_{cut} and [d] tissue having a water content per unit mass, WC.

Equation 6H can be further generalized by expressing the electrical resistance of the cable in terms of the electrical resistivity of the cable material at a nominal temperature of 400 C, a unit length of the cutting cable, L_{cable} and the conduction area of the cable, A_{conduction}. The electrical resistance, R_{cable} of the resistively heated portion of the cutting and pursing cable is the product of the electrical resistivity of the cable, ρ_{cable} and the length of the cable, L_{cable} divided by the cross-sectional area, A_{conduction} Of the current conductor(s) as shown in the following equation:

$${R}_{cable} = {ρ}_{cable} \times ( {L}_{cable} / {A}_{conduction} ) \qquad \{ Equation 6 I \}$$

The total cross-sectional area for conduction of electrical current, A_{conduction} in Equation 6I can be expressed as follows:

$${A}_{conduction} = N \times ( 3.1416 / 4 ) \times {( {D}_{wire} )}^{2} \qquad \{ Equation 6 J \}$$

where N is the number of individual wires 34 in the cutting and pursing cable 33 (as seen, for example in FIG. 9A) and D_{wire} is the diameter of the individual wires (in units of cm). As seen in FIG. 9A, the diameter of the individual wires is also designed by the dimension D₁₅. For the alternative embodiment wherein the cutting and pursing cable comprises a single wire, the value of N in Equation K is unity or 1.

Substituting Equation 6I into Equation 6H provides the equation for the minimum level of substantially constant current required for the cutting of tissue with a cable, or alternatively a single wire, as follows:

$${I}_{cut} = {[ \begin{matrix}( {A}_{conduction} / ( {ρ}_{cable} \times {L}_{cable} ) ) \times 4.186 \times {d}_{water} \times \\ ( {D}_{cable} \times 1.2 ) \times {L}_{cable} \times {R}_{cut} \times WC \times ( {C}_{water} \times Δ T + {LH}_{water} )\end{matrix} ]}^{0.5} \qquad \{ Equation 6 K \}$$

Substituting Equation 6J into Equation 6K provides a generalized equation for the thermal cutting of tissue having [a] known water content, WC, [b] the resistively heated portion of the cutting and pursing cable having known electrical resistivity and dimensions and [c] advancing through tissue at a known cutting rate, R_{cut} as follows:

$${I}_{cut} = {[ \begin{matrix}\begin{matrix}( ( N \times ( 3.1416 / 4 ) \times {( {D}_{wire} )}^{2} ) / ( {ρ}_{cable} \times {L}_{cable} ) ) \times 4.186 \times \\ {d}_{water} ( {D}_{cable} \times 1.2 ) \times {L}_{cable} \times \end{matrix} \\ {R}_{cut} \times WC \times ( {C}_{water} \times Δ T + {LH}_{water} )\end{matrix} ]}^{0.5} \qquad \{ Equation 6 L \}$$

As seen in Equation 6L, the length of the resistively heated portion of the cutting and pursing cable, L_{cable} appears in both the numerator and denominator and therefore cancels from the equation. Hence, the equation for the thermal cutting of tissue having [a] known water content, WC, [b] the resistively heated portion of cutting and pursing cable having known electrical resistivity and dimensions and [c] advancing through tissue at a known cutting rate, R_{cut} seen in Equation 6L is independent of the length of the resistively cutting portion of the cutting and pursing cable. Also, referring to Equation 6L, for the alternative embodiment wherein the cutting and pursing cable comprises a single wire, the value of N in Equation L is 1. After canceling the term L_{cable} that appears in both the numerator and denominator of Equation L, the generalized equation for the thermal cutting of tissue can be expressed, as follows:

$${I}_{cut} = {[ \begin{matrix}\begin{matrix}( ( N \times ( 3.1416 / 4 ) \times {( {D}_{wire} )}^{2} ) / ( {ρ}_{cable} ) ) \times \\ 4.186 \times {d}_{water} \times ( {D}_{cable} \times 1.2 ) \times \end{matrix} \\ {R}_{cut} \times WC \times ( {C}_{water} \times Δ T + {LH}_{water} )\end{matrix} ]}^{0.5} \qquad \{ Equation 6 M \}$$

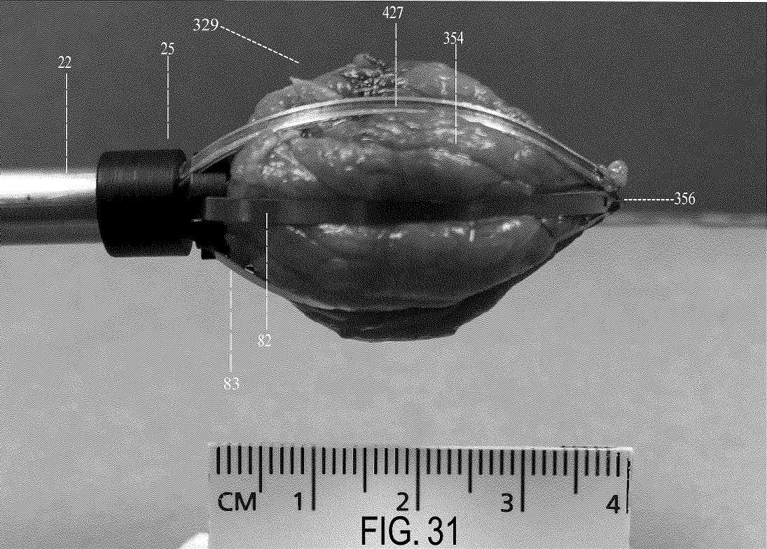

By way of example, the minimum level of substantially constant current, I_{cut} required for the thermal cutting of tissue can be calculated for the case of the actual apparatus and test parameters utilized in laboratory testing performed by the applicant. The actual apparatus, known thermo-physical properties and test parameters utilized in laboratory testing performed by the applicant are as follows: [a] the electrical resistively ρ_{cable} for the Haynes 25 (also known as L605) cable at a nominal tissue cutting temperature of 400 C is 98.5×10⁻⁶ ohm-cm, [b] the diameter of the cable D_{cable} is 0.0099 cm, [c] the diameter of seven individual wires in the cable, D_{wire} is 0.0033 cm, [d] the rate of tissue cutting Rout is 0.23 cm/second, [e] the water content fraction of tissue WC is 70%, [f] the specific heat of water, C_{water} is 1.0 cal/gram-C, [g] the temperature increase, ΔT required to raise the cellular liquid in tissue being thermally cut from body temperature of 37° C. to the boiling of water, 100° C. is 63° C. and [h] the latent heat of vaporization of water in cells being thermally cut is 540 calories/gram Since the cable length, L_{cable} appears in both the numerator and denominator of Equation 6L, this term cancels out and the calculated minimum level of substantially constant current required for the thermal cutting of tissue is independent of the length, L_{cable} of the resistively heated portion of the cutting and pursing cable. Using the above values for all of the parameters on the right side of Equation 6L yields the calculated minimum level of substantially constant current of 1.71 amps that is required for the thermal cutting of tissue. During laboratory testing using a prototype tissue incision and retrieval system 10 as illustrated in FIG. 1 and employed to capture the 25 mm diameter target tissue volume 354 seen in FIG. 31, the measured constant current level during the thermal cutting was 1.87 amps. The close agreement between the calculated minimum level of substantially constant current required for the thermal cutting of tissue and the measured level of constant current during tissue incision and capture validates the generalized equation for the minimum level of substantially constant current required for the thermal cutting of tissue as specified in Equation 6L.

The minimum amount of power required, Pout for the thermal cutting of tissue, as seen in Equation 6E, can also be expressed as the minimum heat flux, P_{cut}/A_{surface} required for the thermal cutting of tissue by dividing both sides of Equation 6E by the surface area, A_{surface} for a known length, L_{cable} of the resistively heated portion of cutting and pursing cable 33. The surface area, Δsurface for a known length, L_{cable} of the resistively heated portion of the cutting and pursing cable 33 is the product of the circumference and length of the resistively heated portion of the cutting and pursing cable and the can be defined as follows:

$${A}_{surface} = 3.1416 \times {D}_{cable} \times {L}_{cable} \qquad \{ Equation 6 N \}$$

Dividing both sides of Equation 6E by the surface area of the cable, A_{surface} provides an equation for the minimum heat flux required for the thermal cutting of tissue can be expressed as follows:

$${P}_{cut} / {A}_{surface} = ( 1 / ( 3.1416 \times {D}_{cable} \times {L}_{cable} ) ) \times 4.186 \times {d}_{water} \times ( {D}_{cable} \times 1.2 ) \times {L}_{cable} \times {R}_{cut} \times WC \times ( {C}_{water} \times Δ T + {LH}_{water} ) \qquad \{ Equation 6 O \}$$

As seen in Equation 60, the cable length, L_{cable} appears in both the numerator and denominator of Equation 60 and therefore this term cancels out and the calculated minimum heat flux, P_{cut}/A_{surface} required for the thermal cutting of tissue is independent of the length, L_{cable} of the resistively heated portion of the cutting and pursing cable. After canceling out the terms for the cable length, L_{cable} in Equation 60, the generalized equation for the minimum heat flux required for the thermal cutting of tissue can be expressed as follows:

$${P}_{cut} / {A}_{surface} = ( 1 / ( 3.1416 \times {D}_{cable} ) ) \times 4.186 \times {d}_{water} \times ( {D}_{cable} \times 1.2 ) \times {R}_{cut} \times WC \times ( {C}_{water} \times Δ T + {LH}_{water} ) \qquad \{ Equation 6 P \}$$

By way of example, the minimum heat flux, P_{cut}/A_{surface} required for the thermal cutting of tissue can be calculated for the case of the actual apparatus and test parameters utilized in laboratory testing performed by the applicant. The actual apparatus, known thermo-physical properties and test parameters utilized in resistively ρ_{cable} for the Haynes 25 (also known as L605) cable at a nominal tissue cutting temperature of 400 C is 98.5×10⁻⁶ ohm-cm, [b] the diameter of the cable D_{cable} IS 0.0099 cm, [c] the diameter of seven individual wires in the cable, D_{wire} is 0.0033 cm, [d] the rate of tissue cutting Rout is 0.23 cm/second, [e] the water content fraction of tissue WC is 70%, [f] the specific heat of water, C_{water} is 1.0 cal/gram-C, [g] the temperature increase, ΔT required to raise the cellular liquid in tissue being thermally cut from body temperature of 37° C. to the boiling of water, 100° C. is 63° C. and [h] the latent heat of vaporization of water in cells being thermally cut is 540 calories/gram. Using the above values for all of the parameters on the right side of Equation 6N yields the calculated minimum heat flux, P_{cut}/A_{surface} of 155 watts/cm² that is required for the thermal cutting of tissue.

As seen above in Table 1 for Test No. 3, the laboratory testing performed to determine the minimum level of constant current required for the thermal cutting of tissue using the same cable diameter, D_{cable} (viz., 0.0099 cm) and rate of cutting, R_{cut} (viz., 0.23 cm/sec.) as used in the above calculation of the minimum heat flux required for thermal cutting, the measured heat flux during laboratory testing was 150 watts/cm². The close agreement between actual laboratory measurement of the actual minimum heat flux required for the thermal cutting of tissue (viz., 150 watts/cm²) and the calculated minimum heat flux required for the thermal cutting of tissue (viz., 155 watts/cm²) provides validation of the generalized Equation 6N for the calculation the minimum heat flux required for the thermal cutting of tissue.

TABLE 2
Calculated Minimum Level of Substantially Constant Current and Minimum Heat
Flux Required for Thermal Cutting of Tissue with Resistively Heated Cable

| | | | Calculated | Calculated | Measured | |
|---|---|---|---|---|---|---|
| | Cable | Rate of | Minimum | Minimum | Level of | Measured |
| | Diameter, | Cutting, | Constant | Heat Flux | Constant | Heat Flux |
| Case | D_{cable} | R_{cut} | Current | Required | Current | Required |
| No. | (inch) | (cm/sec) | (amps) | (watts/cm²) | (amps) | (watts/cm²) |
| 1 | 0.0039 | 0.200 | 1.60 | 135 | | |
| 2 | 0.0039 | 0.230 | 1.71 | 155 | 1.87 | 150 |
| 3 | 0.0039 | 0.275 | 1.87 | 186 | | |
| 4 | 0.0039 | 0.300 | 1.96 | 202 | | |
| 5 | 0.0045 | 0.230 | 1.84 | 155 | | |
| 6 | 0.0045 | 0.275 | 2.01 | 186 | | |
| 7 | 0.0045 | 0.300 | 2.10 | 202 | | |

As discussed above, the mechanism of thermal cutting of tissue is the advancement of a resistively heated cable, operating at a temperature of about 400° C., through soft tissue. The confinement of electric current to flow only within the cable, the cable having an electrical resistance that is orders of magnitude lower than the surrounding soft tissue, effectively avoids the flow of any electrical current into the adjacent tissue. Hence, the thermal cutting of tissue involves only the conduction of heat into the adjacent tissue during tissue cutting. Consequently, due to the small diameter of the cutting cable (e.g., 0.0039 inch) and the rate at which the cable is advanced through the tissue, the small cable surface area in contact with tissue combined with the brief contact period results in a depth of thermal injury at the surface of the captured tissue specimen that is limited to less than about 0.001″ to 0.002″. As a consequence of the very thin layer of thermal injury on the surface of the captured tissue specimen, as defined by the circumscribing tissue capture basket seen in FIG. 25, essentially all of the captured tissue specimen is suitable for post-excision examination by a pathologist and, importantly, the assessment of the extent of malignancy-free (i.e., “clear” or healthy) margins around any identified malignant lesion.

In contrast, electrosurgical cutting of tissue with a wire or cable in prior art devices requires the flow of electrical current from a wire or cable into and through the tissue being incised wherein an electrical arc is formed in the gap between the wire or cable and the tissue as a result of application of a high voltage difference between the wire or cable and the tissue, typically at a level of greater than 1000 volts (peak-to-peak) at a frequency of at least 300 kHz. In the case of electrosurgical cutting to excise and capture a volume of tissue, as specified in U.S. Pat. No. 6,471,659 and incorporated in its entirety herein by reference, the essential flow of electrical current into and through adjacent tissue to achieve tissue cutting may cause unwanted heating of adjacent tissue well beyond the path of tissue cutting that can result in significant thermal damage to nerves and cause pain due to the effect of unwanted current flow in regions of the body beyond the region of applied local anesthesia. The thermal damage to portions of the excised volume of captured tissue is disadvantageous in that the damaged portions of the captured tissue specimen, intended for subsequent examination by a pathologist, are compromised and limit the available portions of the capture tissue specimen suitable for such examination by a pathologist (e.g., assessment of the boundary between malignant and healthy tissue).

The test procedure described above to perform cutting tests performed on samples of ex vivo animal tissue 251 using a solid platinum wire 250, having a nominal diameter of 0.003 inch or 0.005 inch, enabled the estimation of the temperature of a cutting wire or cable during the thermal cutting of tissue at a preferred rate of 2.3 to 2.7 mm/second. Based on the above-described thermal cutting tests in ex vivo animal tissue, the temperature of the cutting wire or cable was calculated to be in the range of 350° C. to 400° C. The resistance per unit length of the first through sixth segments of the resistively heated portion of the cutting and pursing cable 89-94 can next be estimated assuming a cutting cable temperature at the upper end of this range, viz., at 400° C. According to Page 8 of the technical data brochure (Catalog H-3057G available from Haynes International, Kokomo, Indiana) for the cobalt/chromium/tungsten/nickel alloy known as Haynes 25 (also known as L605), the measured electrical resistivity values for Haynes 25 at 20° C. and 400° C. are 88.6 and 98.5×10⁻⁶ ohm-cm, respectively. The electrical resistivity values indicate that the resistance of a Haynes 25 cable exhibits a temperature associated coefficient of 1.201 that results in a cable resistance that is 1.251× higher at 400° C. than at 20° C. (i.e., room temperature).

As stated above, the measured electrical resistance per unit centimeter length for the second preferred design for cutting and pursing cable 33 having a nominal diameter of 0.0039 inch is 1.63 ohm/cm at 20° C. Using the manufacturer supplied electrical resistivity values for L605 at 20° C. and 400° C., the electrical resistance per unit centimeter length measured at 20° C. can be estimated for the cable operating at a temperature of 400° C. (i.e., the expected cable temperature during thermal cutting of tissue) by the product of the electrical resistance at 20° C. (viz., 1.63 ohms/cm) and the temperature associated coefficient of 1.251 resulting in an estimated electrical resistance per unit centimeter length for the first preferred design for cutting and pursing cable 33 of 1.81 ohms/cm at an operating temperature of 400° C.

The required level of the constant current flowing in the first through sixth segments of the resistively heated portion of the cutting and pursing cable 89-94 to achieve thermal cutting of tissue at a rate of 2.3 to 2.7 mm/second can now be estimated. The estimated required constant current level is based on the above measured and derived properties of cutting and pursing cable 33 in combination with the experimentally determined level of the preferred heat flux of 220 watts/cm² emanating from cutting and pursing cable 33 during the thermal cutting of tissue. The required level of constant current required for thermal cutting of tissue can be estimated based the Joule-Lenz Law in combination with the above derived values for the required resistive heating power per unit length, P/L and the electrical resistance per unit length, R/L of the first through sixth segments of the resistively heated portion of the cutting and pursing cable 89-94. The above Equation 3 can be revised to express the required constant current, I_{constant} in terms of the required power dissipation per unit 1.0 cm length, P/L at the preferred heat flux of 220 watts/cm² and the measured electrical resistance adjusted to an operating temperature of 400° C.

$${I}_{constant} = {[ ( P / L ) / ( R / L ) ]}^{1 / 2} \qquad \{ Equation 9 \}$$

For the case of a preferred design for cutting and pursing cable 33 having a nominal diameter of 0.0039 inch and operating at the preferred heat flux of 220 watts/cm², the resistive heater power per unit centimeter length of the first through sixth segments of the resistively heated portion of the cutting and pursing cable 89-94 was determined to be 7.28 watts/cm based on cutting tests at constant current in ex vivo animal tissue, as discussed above. Also, as discussed above, the calculated electrical resistance per unit centimeter length, R/L of the first through sixth segments of the resistively heated portion of the cutting and pursing cable 89-94 was determined to be 1.81 ohms/cm at an operating temperature of 400° C. Based on Equation 9, the required level of constant current, I_{current} to generate a preferred heat flux of 220 watts/cm² within a cutting and pursing cable 33 having a nominal diameter of 0.0030 inch and operating at a temperature of 400° C. is 2.01 amps.

Returning to FIGS. 9, 9A and 11, the required level of constant current, I_{current} derived above for a preferred design for cutting and pursing cable 33, having a diameter of 0.0039 inch confirms the essential requirement for partitioning cutting and pursing cable 33 in two functional regions. As discussed earlier, a first functional region is the first and second tensioning portions of the cutting and pursing cable 118 and 119 that is proximal to the first and second electrically and thermally conductive eyelets 446 and 450, respectively, located at the distal end of the multi-lumen flexible polymeric extrusion assembly 426. A second functional region is the resistively heated portion of the cutting and pursing cable that is immediately distal to the electrically and thermally conductive eyelets 446 and 450 located at the distal end of the multi-lumen flexible polymeric extrusion assembly 426. This second functional region comprises the first through sixth segments of the resistively heated portion of the cutting and pursing cable 89-94. Returning to FIGS. 11, the demarcation or boundary between these two functional regions is seen at first and second transition boundaries 396 and 406 that occur at the electrically and thermally conductive eyelets 446 and 450, respectively, located at the distal end of the multi-lumen flexible polymeric extrusion assembly 426. Accordingly, the applied constant current is electrically conducted only through those the first through sixth segments of the resistively heated portion of cutting and pursing cable 89-94, respectively, that are distal to their point of sliding electrical contact with first and second electrically and thermally conductive eyelets 446 and 450 that serve as electrodes. Consequently, electrical current is conducted only into the first through sixth segments of the resistively heated portion of cutting and pursing cable 89-94, respectively, that are distal to eyelets 446 and 450 and in direct contact with tissue. The Joulean heating generated within each of the first through sixth segments of the resistively heated portion of the cutting and pursing cable 89-94 is dissipated into the tissue being incised, primarily through the process of the vaporization of the water contained within and comprising about 70% of the mass of each cell as discussed above. As a consequence of the water vaporization-based heat dissipation mechanism associated with the cutting of tissue by the passage of a constant current flowing through the first through sixth segments of the resistively heated portion of the cutting and pursing cable 89-94, the temperature of the first through sixth segments of the resistively heated portion of the cutting and pursing cable 89-94 increases to a maximum of only about 400° C. through Joulean or resistive heating that has been experimentally determined to be about 7.28 watts per centimeter of length.

The levels of constant current required for the thermal cutting of tissue by the first through sixth segments of the resistively heated portion of the cutting and pursing cable 89-94 in direct contact with tissue would result in very high temperatures if the constant current levels were required to flow in the first and second tensionable portions of the cutting and pursing cables 118 and 119, respectively, since these tensionable portions of the cutting and pursing cable are not in contact with tissue but are surrounded by a thermally insulative layer of air and/or a thermally insulative interior wall of a lumen of the multi-lumen flexible polymeric extrusion 420 or 508. Due to the low thermal conductivity of the air that surrounds the first and second tensionable portions of the cutting and pursing cables 118 and 119, respectively, the largest portion of the heat dissipation from the surface of the cutting and pursing cable 33 in the tensionable portions of the cutting and pursing cables would be heat dissipation by radiation heat transfer. According to the Stefan-Boltzmann Law, assuming the maximum radiation emittance of 1.0 from the surface of cutting and pursing cable 33 (i.e., also known as black body radiation), radiated heat flux, W_{radiation} can be expressed as the fourth power of absolute temperature, T_{absolute} as seen below in Equation 10 (McAdams, W., Heat Transmission. 1954; McGraw-Hill Book Company, New York: 59-60).

$${W}_{radiation} = σ \times {T}_{absolute}^{4} \qquad \{ Equation 10 \}$$

where σ is the Stefan-Boltzmann constant equal to 5.67×10⁻¹² watts/cm²-K⁻⁴ and the absolute temperature is the temperature in units of ° K or temperature in ° C. plus 273° C. Since the preferred heat flux of 220 watts/cm² has been established based on cutting tests in ex vivo animal tissue, as described above, and the associated constant current levels required to achieve the preferred heat flux for the two preferred diameters of cutting and pursing cable 33, Equation 10 can solved for the estimated cable temperature if the same heat flux of 220 watts/cm² is required to be dissipated for the case in which the cutting and pursing cable 33 is in air and not in contact with tissue.

$${T}_{absolute} = {[ {W}_{radiation} / σ ]}^{1 / 4} \qquad \{ Equation 11 \}$$

Based on operation at a constant current level sufficient to generate the preferred heat flux required for thermal cutting of tissue, the calculated temperature assuming black body radiation (i.e., the maximum achievable radiation heat transfer) is 2,496° K or 2,223° C. This calculated temperature is more than 800° C. higher than the melting point of any metal alloy (L605 alloy or austenitic stainless steel alloys) that could be selected for the manufacture of cutting and pursing cable 33. In addition, such high temperatures in electrical leads would result in significant thermal damage and the melting of plastic components with the tissue incision and retrieval assembly 12. Hence, the present disclosure specifies apparatus and methods that are essential for the conduction of constant current only through low-resistance electrically conductive paths that extends from the current source within circuit board assembly 184 to the location that the cutting and pursing cable 33 is in sliding electrical contact with first and second electrically and thermally conductive eyelets 446 and 450, respectively.

By way of example, the low-resistance electrical current flow paths are seen in FIG. 17 as first and second electrically and thermally conductive leads 444 and 448, respectively, that extend between [a] first and second electrically and thermally conductive eyelets 446 and 450, respectively and [b] first proximal lead wire 114 with electrically insulative covering 115 and second proximal lead wire 116 with electrically insulative covering 117, respectively.

In a first embodiment and still referring to FIG. 17, first electrically and thermally conductive eyelet 446 and integral first electrically and thermally conductive lead 444 are fabricated using solid, substantially pure silver wire containing at least 99.5% silver and having a diameter of 0.008 to 0.014 inch. Likewise, in the same embodiment, second electrically and thermally conductive eyelet 450 and integral second electrically and thermally conductive lead 448 are fabricated using solid, substantially pure silver wire containing at least 99.5% silver and having a diameter of 0.008 to 0.014 inch. The solid, substantially pure silver wire containing at least 99.5% silver that comprises first and second electrically and thermally conductive eyelets 446 and 450 as well as first and second electrically and thermally conductive leads 444 and 448 minimizes resistive heating while providing a thermally conductive path to withdraw heat that is transferred to the eyelets at point of contact with the resistively heated portions cutting and pursing cables 89 and 94, respectively. Another attribute of 99.5% pure silver wire is its established biocompatibility.

Still referring to FIGS. 11 and 17, the solid, substantially pure silver wire (containing at least 99.5% silver) that comprises first and second electrically and thermally conductive leads 444 and 448 extend through and along the full length of first and fourth lumens, 434a and 442a, respectively, of multi-lumen flexible polymeric extrusion member 420. By way of example, a preferred elastomer for the multi-lumen flexible polymeric extrusion member 420 is Polyamide 12 (also known as Nylon 12) with a medical-grade extrusion resin known as Rilsamid MED Polyamide 12, having a modulus of elasticity of about 12×10⁶ psi and manufactured by Foster Polymers (Putnam, Connecticut). The modulus of elasticity of the silver comprising first and second electrically and thermally conductive leads 444 and 448, being about 10.9×10⁶ psi, adds to the column strength of the multi-lumen flexible polymeric extrusion member 420. The multi-lumen flexible polymeric extrusion member 420 in combination with first and second electrically and thermally conductive leads 444 and 448 (in the form of solid, substantially pure silver wires) provides the required level of column strength to enable advancement of the multi-lumen flexible polymeric extrusion assembly 426 without buckling within the unsupported length between the leaf member and multi-lumen flexible polymeric extrusion assembly support member 400 and the distal end assembly 25 of delivery cannula 22 as seen in FIGS. 12 and 26.

Referring now to FIGS. 8, 9, 11 and 17, first and second proximal lead wires 114 and 116 are preferably flexible, multi-strand, electrically conductive 28-gauge copper wires with an electrically insulative covering 115 and 117, respectively (e.g., polyvinyl chloride insulative covering). The first and second proximal lead wires 114 and 116 extend from the proximal end of the first and second electrically and thermally conductive leads 444 and 448 to first and second electrical contacts 120 and 122 located on the right side and left side, respectively, of single-use support housing 100 as seen earlier in FIG. 8. The first and second contacts 120 and 122 in combination with first and second proximal lead wires 114 and 116, respectively and first and second electrically and thermally conductive leads 444 and 448, respectively, provide electrical current flow path 399. The electrical current flow path 399 represents the pathways within the single-use support housing that supply constant current from constant current source 247 located on circuit board assembly 184 to first and second electrically and thermally conductive eyelets 446 and 450, respectively.

Still referring to FIGS. 9, 11 and 17, constant current is supplied to first and second electrically and thermally conductive eyelets 446 and 450 that are in electrical communication with first and sixth segments of the resistively heated portion of the cutting and pursing cable 89 and 94, respectively, through the first and second sliding electrical contacts 396 and 406, respectively, at the interface between the first and second electrically and thermally conductive eyelets 446 and 450, respectively and the respective first and sixth segments of the resistively heated portion of the cutting and pursing cable 89 and 94, respectively, seen at the distal end of multi-lumen flexible polymeric extrusion assembly 426 as seen in FIGS. 11 and 17. The interface between [a] first and second electrically and thermally conductive eyelets 446 and 450 and [b] first and sixth segment of the resistively heated portion of the cutting and pursing cables 89 and 94, respectively, is seen in FIG. 11 at first and second transition boundaries, 396 and 406, respectively. The first and second transition boundaries, 396 and 406 designate the locations at which constant current begins to flow from the first and second electrically and thermally conductive eyelets 446 and 450, respectively, and into the first and sixth segments of the resistively heated portion of the cutting and pursing cable 89 and 94, respectively.

Referring now to FIGS. 9, 11 and 21, the flow of constant current in the first through sixth segments of the resistively heated portion of cutting and pursing cable 89-94 is illustrated electrical current flow path 399. As seen in the end view at various stages of deployment of tissue capture basket 326 in FIGS. 9 and 11, electrical current flow path 399 continues sequentially from first electrically and thermally conductive eyelet 446 (located at distal end of multi-lumen flexible polymeric extrusion assembly 426) through the first segment of the resistively heated portion of the cutting and pursing cable 89, through the second segment of the resistively heated portion of the cutting and pursing cable 90, through the third segment of the resistively heated portion of the cutting and pursing cable 91, through the fourth segment of the resistively heated portion of the cutting and pursing cable 92, through the fifth segment of the resistively heated portion of the cutting and pursing cable 93, through the sixth segment of the resistively heated portion of the cutting and pursing cable 94 and finally into second electrically and thermally conductive eyelet 450.

Returning to FIGS. 4, 6, 8 and 9 in combination with FIGS. 12, 20, 21 and 26-28, the discourse that follows specifies the method of deploying five leaf members 82-86 and multi-lumen flexible polymeric extrusion assembly 426 to form first through sixth segments of the resistively heated portion of the cutting and pursing cable 89-94, respectively, the latter being supported at the distal ends of the leaf members 82-86 and multi-lumen flexible polymeric extrusion assembly 426 as described earlier with regard to FIGS. 11 and 17. Referring first to FIG. 4, axially displaceable drive assembly drive member 324 and cable mounting hub 296 disposed within single-use support housing 100 are slideably disposed on support tube 282 that is aligned with longitudinal axis 8 as seen in FIG. 2. By way of example, the proximal end of support tube 282 may be secured to the proximal end face 64 of single-use support housing 100 by the combination of threaded rod 177 swaged within the proximal end of support tube 282 and support tube tensioning nut 63 as seen in FIG. 4. As seen in FIGS. 4, 8 and 12, support tube 282 extends the full length, L6 of single-use support housing 100 beginning at the proximal end face 64 of single-use support housing 100 and ending at tip component 266 at the distal end of delivery cannula 22. By way of example, support tube 282 may be drawn and welded stainless steel 304 tubing having an outside diameter of 0.083 inch, inside diameter of 0.063 inch and available from Vita Needle Company (Needham, Massachusetts). By way of further example, the distal end of support tube 282 may be deformed resulting in flared end 346 that retains tip component 266 as seen in FIG. 12.

As specified earlier in this disclosure with reference to FIGS. 3 and 4, drive assembly drive member 324 is advanced forwardly by the first pivotable drive finger 185a extending from translation nut 182a as it is driven forward by motor-actuated drive tube drive member translation assembly 180a. As seen in FIG. 3, motor-actuated drive tube drive member translation assembly 180a disposed within handpiece assembly 15 is comprised of motor and planetary gear train assembly 170a, flexible metallic bellows-shaped coupler 174a, lead screw 176a, translation nut 18a and thrust bearing 171a.

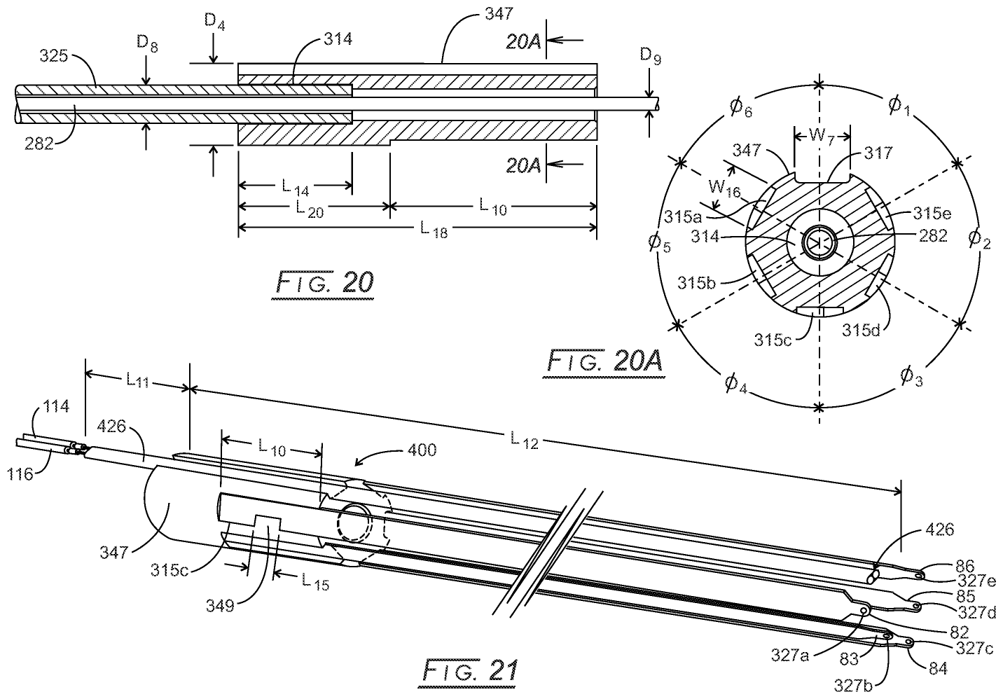

Referring now to FIGS. 4, 5, 20, 21 and 26, as drive assembly drive member 324 is driven in forward direction 476, drive tube 325, secured to drive assembly drive member 324, induces corresponding and proportionate axial forward displacement of leaf member and extrusion assembly support member 347. By way of example, drive tube 325 may be drawn and welded stainless steel 304 tubing with an outside diameter of 0.109 inch, inside diameter of 0.095 inch and available from Vita Needle Company (Needham, Massachusetts). As seen in FIG. 20, the distal end of drive tube 325 is received in circular opening at proximal end of leaf member and extrusion assembly support member 347 to effect forward corresponding advancement of leaf member and extrusion assembly support member 347 as drive tube 325 is advanced forwardly by motor-actuated drive tube drive member translation assembly 180a. As seen in FIGS. 20A and 21, first through fifth leaf members 82-86, respectively and multi-lumen flexible polymeric extrusion assembly 426 are disposed on the perimeter of leaf member and extrusion assembly support member 347. By way of example, leaf member and extrusion assembly support member 347 may be an electrically insulative, commonly injection-molded polymer such as polycarbonate. The first through fifth leaf members 82-86 may be adhesively bonded within first through fifth leaf member support cavities 315a-315e, respectively, using an adhesive such as cyanoacrylate. The multi-lumen flexible polymeric extrusion assembly 426 may also be adhesively bonded within multi-lumen flexible polymeric extrusion support cavity 317.

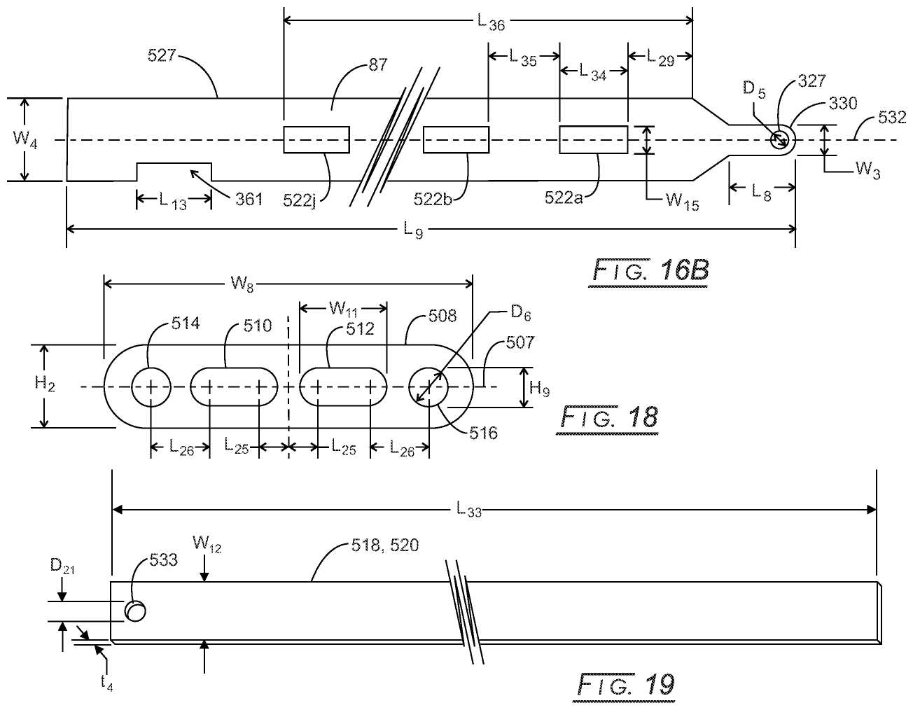

As seen in FIGS. 16, 20A, 21 and 25, retaining member 349 is included within each leaf member support cavity 315a-315e that matches corresponding leaf member retaining notch 361 to ensure secure attachment between leaf members 82-86 and leaf member and extrusion assembly support member 347. The retaining member 349 present in first through fifth leaf member support cavities 315a-315e ensures that the adhesive bond between first through fifth leaf members 82-86 and leaf member and extrusion assembly support member 347 can withstand the longitudinal forces required to withdraw the tissue cutting and capture assembly 329 comprising first through fifth leaf members 82-86 through the cannula distal end assembly 25 during the assembly process, as seen in FIG. 12.

By way of example of a first embodiment of the present disclosure and referring to FIG. 16, first through fifth leaf members 82-86 may be photochemically machined from a thin stainless steel sheet (e.g., full-hard stainless steel Type 304) having a thickness, t1 of about 0.002 inch to 0.007 inch (e.g., Photofabrication Engineering, Inc., Milford, Massachusetts). The first through fifth leaf members 82-86 are identical in thickness and shape, having a widthwise extent, W₄ of about 0.060 inch to 0.080 inch and lengthwise extent, L₉ of about 2.8 to 3.5 inches as shown in FIG. 16 for first leaf member 82. The first through fifth leaf members 82-86 are covered by a thin, flexible, biocompatible electrically insulative coating (e.g., chemically vapor deposited Parylene HT applied by Specialty Coating Systems, Indianapolis, Indiana). The preferred Parylene HT electrically insulative coating is capable of withstanding temperatures of up to at least 400 C and prevents unwanted electrical current flow between the leaf members during the application of constant current to the first through sixth segments of the resistively heated portion of the cutting and pursing cable 89-94. Preferably, the thickness of the electrically insulative coating applied to all exterior surfaces of the first through fifth leaf members, 82-86 is in the range from 0.00015 inch to 0.00050 inch, preferably about 0.00020 inch.

By way of example of a second embodiment of the present disclosure and referring to FIG. 16B, first through fifth perforated leaf members 527-531 may be photochemically machined from a thin stainless steel sheet (e.g., full-hard stainless steel Type 304) having a thickness, t1 of about 0.002 inch to 0.007 inch. The first through fifth perforated leaf members 527-531 are identical in thickness and shape, having a widthwise extent, W₄ of about 0.060 inch to 0.080 inch and lengthwise extent, L9 of about 2.8 to 3.5 inches as shown in FIG. 16B for first perforated leaf member 527. Unlike leaf member 82 seen in FIG. 16, perforated leaf member 527 preferably includes 10 rectangular perforations 522a-522j that are centered along the longitudinal centerline 532 of each perforated leaf member 527-531 as seen for perforated leaf member 527 in FIG. 16B. As seen in FIG. 16B, the distal edge of most distal rectangular perforation 522a is positioned a distance L₂₉ from the shoulder of the eyelet containing tip 330 of perforated leaf member 527. A uniform spacing, L₃₅ is maintained between the perforations and ranges from 0.100 to 0.160 inch. The width, W₁₅ of all perforations 522a-522j ranges from 0.025 to 0.035 inch and the length, L₃₄ of all perforations 522a-522j ranges from 0.190 to 0.230 inch. The spacing and size of the rectangular perforations 522a-522j in leaf members 527-531 have been optimized based on the principles of cantilever beam structural analysis to achieve two functional objectives. A first functional objective of the dimensions of the perforated leaf members 527-531 is to attain sufficient stiffness of the first through fifth leaf members 527-531 to maintain the preferred deployment angle, @2 of about 45° as seen in FIG. 25 in the presence of the tension that exists in the first through sixth segments of the resistively heated portion of the cutting and pursing cable 89-94 due to cable friction as it traverses through the tissue being incised and as the cable passes through the eyelets of the leaf members 82-86 and the multi-lumen flexible polymeric extrusion member 426. A second functional objective of the dimensions of the perforated leaf members 527-531 is to reduce the pressure applied to the tissue specimen and thereby maintain the integrity of the tissue specimen as the distal ends of the leaf members 527-531 are pursed down through the tension applied to the first through sixth segments of the resistively heated portion of the cutting and pursing cable 89-94.

Referring now to FIGS. 10, 12, 15, 15A and 21, the leaf member and multi-lumen flexible polymeric extrusion assembly 400 is shown in greater detail in cross-section form (as seen in FIG. 10) at the forward end 27 of delivery cannula 22 (as seen in FIG. 12). A detailed cross-sectional view of multi-lumen flexible polymeric extrusion member 420 is seen in FIG. 10 including dimensional references as well as the distance between the lumens and the overall dimensions of the multi-lumen extrusion member 420. In a preferred embodiment, multi-lumen flexible polymeric extrusion member 420 an extruded polymer such as Polyamide 12 (also known as Nylon 12). By way of example, the polymer may be a medical-grade extrusion resin known as Rilsamid MED Polyamide 12, having a modulus of elasticity of about 12×10⁶ psi and manufactured by Foster Polymers (Putnam, Connecticut). By way of example, multi-lumen extrusion member 420 seen in FIG. 10 was extruded by Nordson Medical, Design and Development LLC located in Marlborough, Massachusetts.

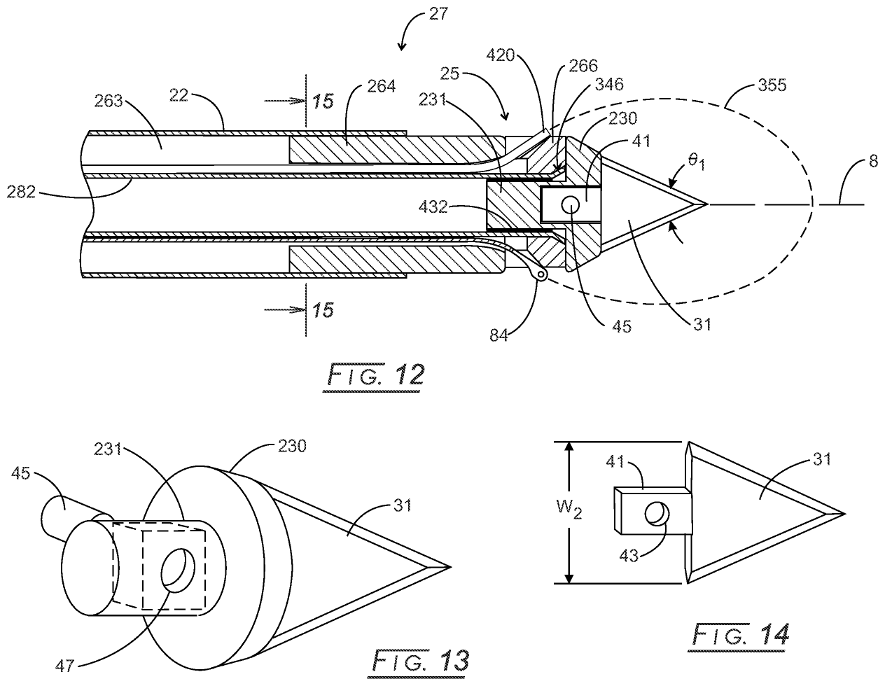

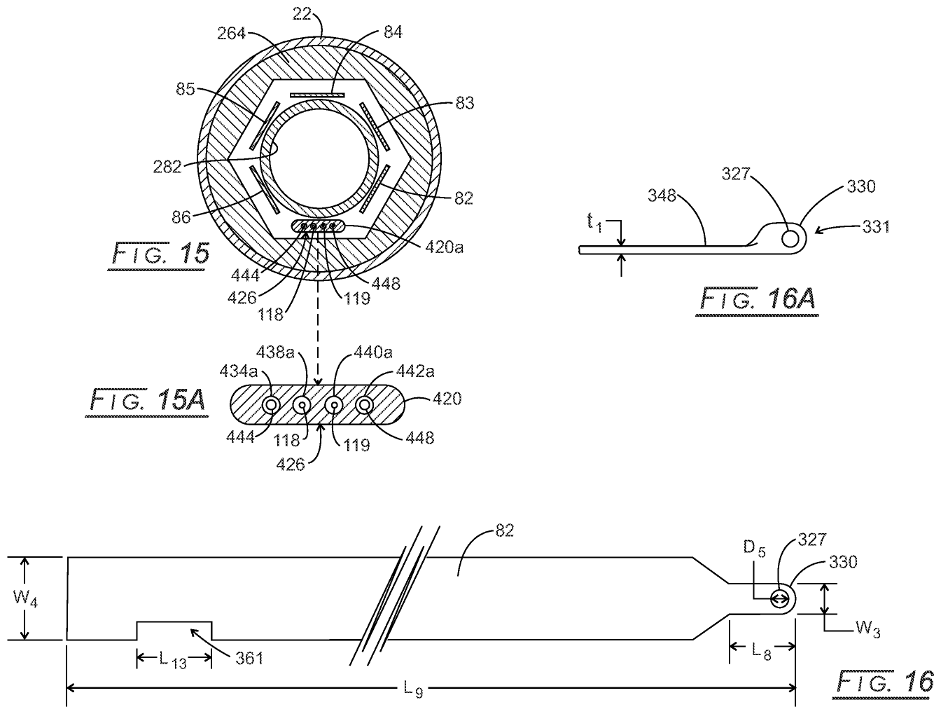

Still referring to FIG. 15 that represents the cross-sectional view as seen in FIG. 12, first through fifth leaf members 82-86 and multi-lumen flexible polymeric extrusion assembly 426 are slideably disposed within the annular hexagonal space defined by the exterior surface of support tube 282 and the interior surface of confinement sleeve tip 264. As seen in FIGS. 12 and 15, the proximal end of confinement sleeve tip 264 is inserted within and supported by the distal end of delivery cannula 22. By way of example, delivery cannula 22 may be stainless steel 304 tubing having an outside diameter of 0.259 inch and inside diameter of 0.238 inch and commercially available from Vita Needle Company (Needham, Massachusetts). Also, by way of example, confinement sleeve tip 264 may be injection molded using a polymer capable of operating at temperatures as high as 200° C. such as polyetherimide (e.g., Ultem 1000 available from Sabic Americas, Inc., Houston, Texas). As seen in FIGS. 12, 22 and 23, tip component 266 may also be injection molded using a polymer capable of operating at temperatures as high as 200° C. such as polyetherimide (e.g., Ultem 1000 available from Sabic Americas, Inc., Houston, Texas).

As seen in FIGS. 15 and 15A, the first and second tensionable portions of cutting and pursing cables 118 and 119 are slideably disposed within the interior of multi-lumen flexible polymeric extrusion assembly 426 at second and third lumens 438a and 440a, respectively, within multi-lumen flexible polymeric extrusion member 420. Also, first and second electrically and thermally conductive leads 444 and 448, respectively, are located within the interior of multi-lumen flexible polymeric extrusion assembly 426 at first and fourth lumens 434a and 442a, respectively, within multi-lumen flexible polymeric extrusion member 420.

Referring to the cross-sectional view in FIG. 12, only leaf member 84 and multi-lumen flexible polymeric extrusion member 420 are seen. Although not shown in cross-sectional view in FIG. 12 but seen in FIG. 21, leaf members 82-83 and 85-86 along with leaf member 84 and multi-lumen flexible polymeric extrusion member 420 are advanced within interior channel 263 of delivery cannula 22 by the previously specified motor-actuated drive tube drive member translation assembly 180a (as seen in FIG. 3). As leaf members 82-86 and multi-lumen flexible polymeric extrusion member 420 are advanced within interior channel 263 of delivery cannula 22, their distal ends follow a locus of movement line 355. The completion of advancement of leaf members 82-86 and multi-lumen flexible polymeric extrusion member 420 along the locus of movement line 355 defines the tissue capture basket 326 seen in FIG. 25 as well as the incised target tissue volume 354 located within the tissue capture basket 326.

Alternatively, as seen in FIG. 12, the locus of movement line 355 defining tissue capture basket 326 can be seen in the end view of the tissue capture basket 326 in FIG. 9 at four different stages of expansion. The four different stages of expansion seen in FIG. 9 are the result of the advancement of the first through fifth leaf members 82-86 along with the multi-lumen flexible polymeric extrusion members 420. Upon achieving the fourth and maximum stage of expansion, the tissue capture basket 326 begins to purse down or contract and continues through the three stages in reverse order (viz., stages labeled with suffixes c, b and a).

In a preferred construction of the tissue incision and retrieval assembly 12 of the present disclosure seen in FIGS. 4 and 17, equal lengths of cutting and pursing cable 33 (seen in cross-sectional view in FIG. 9A) comprise the first and second tensioning portions of cutting and pursing cables 118 and 119. Also, substantially equal lengths of cutting and pursing cable 33 comprise the first through sixth segments of the resistively heated portion of the cutting and pursing cable 89-94, respectively. In this preferred construction, as seen in FIG. 9, five leaf members 82-86 combine with the multi-lumen flexible polymeric extrusion assembly 420 to form a hexagonal-shaped tissue capture basket 326 at each stage of expansion and contraction of the distal ends of the leaf members and multi-lumen flexible polymeric extrusion assembly.

Referring to FIGS. 4, 5, 9, 17 and 21, the cutting and pursing cable 33 comprises, sequentially, first tensioning portion of the cutting and pursing cable 118, first through sixth segments of the resistively heated portion of the cutting and pursing cable 89-94 and second tensioning portion of the cutting and pursing cable 119. The first tensioning portion of the cutting and pursing cable 118 begins at the electrically insulative cable mounting hub 296 located at the proximal end of single-use support housing 100, where first tensioning portion of cutting and pursing cable 118 is secured by first cable fastening machine screw 487 in combination with first cable fastening washer 489. First tensioning portion of the cutting and pursing cable 118 continues forwardly to enter lumen 438a at proximal end of multi-lumen flexible polymeric extrusion assembly 426 and continuing forwardly to and through aperture of first electrically and thermally conductive eyelet 446. Beyond first electrically and thermally conductive eyelet 446, cutting and pursing cable 33 continues from first electrically and thermally conductive eyelet 446 to eyelet 327a of first leaf member 82 to form first segment of the resistively heated portion of the cutting and pursing cable 89 as seen in FIG. 9. Beyond eyelet 327a of first leaf member 82, cutting and pursing cable 33 continues as second segment of the resistively heated portion of the cutting and pursing cable 90 (as seen in FIG. 9) until cutting and pursing cable 33 reaches the eyelet 327b of second leaf member 83 and continuing in like manner to third through eyelets 327c, 327d and 327e of third through fifth leaf members 84, 85 and 86, respectively. Beyond eyelet 327e of fifth leaf member 86, sixth segment of the resistively heated portion of the cutting and pursing cable 94 continues to second electrically and thermally conductive eyelet 450. Beyond and proximal to second electrically and thermally conductive eyelet 450, cutting and pursing cable 33 continues as the second tensionable portion of cutting and pursing cable 119 until it is secured to the electrically insulative cable mounting hub 296 located at the proximal end of single-use support housing 100. The second tensionable portion of cutting and pursing cable 119 is secured to the electrically insulative cable mounting hub 296 by second cable fastening machine screw 488 in combination with first cable fastening washer 490.

Turning briefly to FIGS. 12-14 and 25, surgically sharp blade 31 is seen mounted within blade support 230 and secured at base of blade support 231 with locking pin 45 that extends through a mounting hole 47 located on either side of base of blade support 231 as well as blade shank hole 43 located in shank of blade 41. As seen earlier with regard to tissue incision and retrieval system 10 seen in FIG. 1, surgically sharp blade 31 is mounted at forward end 27 of delivery cannula 22 to enable the advancement of delivery cannula 22 through soft tissue through incision of the soft tissue, the advancement proceeding along the line of the longitudinal axis 8 of delivery cannula 22 in order that cannula distal end assembly 25 is in confronting adjacency to target tissue volume 354 as seen in FIG. 25.

By way of example, surgically sharp blade 31 seen in FIG. 14 may be manufactured by stamping unsharpened blade blanks similar to profile seen in FIG. 14. The stamping is used to form blade blanks from unhardened or annealed stainless steels sheets such as martensitic stainless steel 420 or GIN-5 stainless steel (GIN-5 available from Hitachi Metals America, Ltd., Arlington Heights, Illinois). Following a stamping operation, blade blanks are heat treated to a hardness level of preferably at least 55 Rockwell C scale. The stainless steel sheets preferably have a thickness in the range from 0.020 to 0.030 inch. Following heat treatment and hardening of blade blanks, the blade blanks are sharpened using mechanical grinding and honing processes and/or electrochemical sharpening and honing.

By way of example and returning to FIGS. 12 and 13, tip component 266 may be manufactured by injection molding a high-strength polymeric material such as polyetherimide (e.g., Ultem 1100 resin commercially available from Sabic Americas, Inc., Houston, Texas). Alternatively, tip component 266 seen in FIGS. 12 and 13 may be manufactured by metal injection molding (MIM) a material like stainless steel 304 or stainless steel 17-7PH to provide a tip component 266 having greater mechanical strength characteristics as compared with injection molded polymers.

The mechanism of thermal cutting of tissue using a resistively heated wire or cable allows only the conduction of heat into adjacent tissue and avoids any flow of electrical current into the adjacent tissue. As a consequence, the depth of unwanted thermal injury at the surface layer of the captured tissue specimen is limited to less than about 0.001″ to 0.002″. In contrast, electrosurgical cutting of tissue with a wire or cable in prior art devices requires the flow of electrical current from the wire or cable into and through the tissue being incised wherein an electrical arc is formed in the gap between the wire or cable and the tissue as a result of application of a high voltage difference between the wire or cable and the tissue, typically at a level of greater than 1000 volts (peak-to-peak) at a frequency of at least 300 kHz. In the case of electrosurgical cutting to excise and capture a volume of tissue, as specified in U.S. Pat. No. 6,471,659 and incorporated herein by reference, the essential flow of electrical current into and through adjacent tissue to achieve tissue cutting may cause unwanted heating of adjacent tissue well beyond the path of cutting resulting in thermal damage to portions of the excised volume of captured tissue. The thermal damage to portions of the excised volume of captured tissue are disadvantageous in that the damaged portions of the captured tissue specimen, intended for subsequent examination by a pathologist, are compromised and limit the available portions of the capture tissue specimen suitable for such examination (e.g., assessment of the boundary between malignant and healthy tissue).

Referring to FIG. 26, a partial sectional view presented in connection with FIG. 8 is reproduced wherein tissue incision and retrieval assembly 12 is seen with blade 31 positioned at skin surface of patient 365 prior to advancement of delivery cannula 22 along its path of insertion 363. By way of example, a shallow skin incision is initially performed by the practitioner at skin site 24 to enable the initial insertion of the tissue incision and retrieval assembly 12 as well as the withdrawal of the tissue incision and retrieval assembly 12 along with the captured target tissue volume 354 as seen in FIG. 25. Next, the surgically sharp blade 31 attached to the cannula distal end assembly 25 is used to incise healthy tissue 366 during the advancement of delivery cannula 22 of tissue incision and retrieval assembly 12 along path of insertion 363. By way of example, the advancement of delivery cannula 22 of tissue incision and retrieval assembly 12 may be performed by the practitioner with the aid of real-time ultrasound imaging guidance, MRI guidance or stereotactic radiographic guidance (not shown) based on image-based detection of the location of the target lesion such as a suspicious potentially malignant lesion 368 seen in FIG. 26.

As seen in FIG. 26A, the delivery cannula 22 of tissue incision and retrieval assembly 12 is advanced along path of insertion 363 (previously seen in FIG. 26) until the cannula distal end assembly 25 is just proximal and adjacent to target tissue volume 354 containing, for example, suspicious potentially malignant lesion 368. Once the cannula distal end assembly 25 of tissue incision and retrieval assembly 12 is just proximal and adjacent to target tissue volume 354, the incision of healthy tissue 366 and capture of target tissue volume 354 containing, for example, suspicious potentially malignant lesion 368, commences. As seen in FIG. 26A, cable mounting hub 296, drive assembly drive member 324 as well as leaf member and multi-lumen flexible polymeric extrusion assembly 400 are shown in their initial positions A, B and C as seen at 401, 411 and 421, respectively. The initial positions A, B and C correspond to the positions of these component prior to the start of tissue cutting and capture after the tissue cutting and capture assembly 329 has been positioned adjacent to target tissue volume 354.

Referring next to FIG. 27, a partial sectional view presented in connection with FIGS. 3, 4 and 8 is reproduced wherein, following commencement of the tissue cutting and capture process as described above, first pivotable drive finger 185a mounted on translation nut 182a advances the position of the drive assembly drive member 324 from position B to position B′ as seen at 411 and 412, respectively, in the direction indicated by first movement direction of drive assembly drive member 324 as seen at arrow 413. The advancement of the position of the drive assembly drive member 324 from position B to position B′ as seen at 411 and 412, respectively, and associated advancement of drive tube 325 induces the advancement of leaf member and multi-lumen flexible polymeric extrusion assembly 400 from position C to position C′ as seen at 421 and 422, respectively, indicated by first movement direction of leaf member and multi-lumen flexible polymeric extrusion assembly 400 as seen at arrow 423. In addition, the advancement of the position of the drive assembly drive member 324 from position B to position B′ as seen at 411 and 412, respectively, induces the advancement of electrically insulative cable mounting hub 296 from position A to A′ as seen at 401 and 402, respectively, as indicated by first movement direction of cable mounting hub 403 as a result of the associated advancement of the first and second tensionable portions of cutting and pursing cables 118 and 119, respectively, that are secured to the cable mounting hub 296 as seen in FIG. 5 and discussed previously.

Referring now to FIGS. 9, 21, 26A and 27, tissue cutting and capture assembly 329 has advanced from it its initial position D within cannula distal end assembly 25 to a partially deployed tissue capture basket 326 at a position D′ representing the maximum opening of the tissue cutting and capture assembly 329 as seen at 391 and 392, respectively, as indicated by first movement direction arrow 393.

Referring now to FIGS. 3, 4, 9 and 27, at position 392 (as seen at D′ in FIG. 27) of the partially deployed tissue capture basket 326 corresponding to the maximum opening of the tissue cutting and capture assembly 329, cable mounting hub 296 abuts second pivoting drive finger 185b (not shown in FIG. 27). Upon cable mounting hub 296 advancing to and abutting the position of second pivoting drive finger 185b, the tensioning portions of cutting and pursing cable 118 and 119 become taught thereby initiating the process of pursing down of the deployed tissue basket 326.

Referring next to FIGS. 3, 4, 8 and 28, a partial sectional view presented in connection with FIG. 8 is reproduced wherein, following attainment of the maximum opening of the tissue cutting and capture assembly 329, first pivotable drive finger 185a mounted on translation nut 182 continues to advance the position of the drive assembly drive member 324 from position B′ to position B″ as seen at 412 and 414, respectively, in the direction indicated by second movement direction of drive assembly drive member 324 as seen at arrow 415. Once the maximum opening of the capture basket has been attained as seen at position D′ in FIG. 27 as defined by first and second tensioning cables 118 and 119 become constrained as the cable mounting hub 296 abuts the second pivoting drive finger 185b, secured to second transition nut 182b as part of second motor-actuated cable mounting hub translation assembly 180b, then the combination of the further advancement of drive assembly drive member 324 from position B′ to position B″ as seen at 412 and 414, respectively and the associated advancement of leaf member and multi-lumen flexible polymeric extrusion assembly 400 from C′ to position C″ as seen at 422 to 424, respectively, causes the pursing down of tissue cutting and capture assembly 329 until a substantially single pursed down point 356 is reached. In a preferred embodiment, constant current supply 247 within circuit board assembly 184 continuously measures the voltage difference between first and second constant current connector pins 51 and 53, respectively. A measured voltage difference between the first and second constant current connector pins 51 and 53, respectively, that is less than a pre-selected lower limit value (e.g., 2.0 volts for the case of an applied constant current level of 2.0 amps) indicates that the distal ends of leaf members and distal ends of multi-lumen flexible polymeric extrusion assembly have been pursed down to the maximum possible extent as illustrated in FIG. 28 at single purse down point 356. Once the measured voltage decreases below a pre-selected lower limit value, the application of constant current being applied to the first through sixth segments of the resistively heated portion of the cutting and pursing cable 89-94, respectively, is terminated and the tissue incision and capture process is determined to be complete. At the detected completion of the tissue incision and capture process the capture “Complete” indicator light 52 is illuminated on handpiece assembly 15 as described earlier with regard to FIG. 1. Simultaneously, the voltage applied to first motor 170a and second motor 170b is terminated. Also and simultaneously, the audible tone being generated by speaker 200 within circuit board assembly 184 of internal energy source and control system 181, as seen in FIG. 3, to alert the practitioner that constant current is being applied is discontinued.

A seen in FIG. 28, the advancement and purse down of tissue cutting and capture assembly 329 circumscribes the target tissue volume 354 containing, for example, suspicious potentially malignant lesion 368. Preferably, the region between the perimeter surface of the suspicious potentially malignant lesion 368 and the circumscribing boundary of target tissue volume 354 contains a margin or boundary layer of healthy tissue 367. The first through fifth leaf members 82-86 in combination with multi-lumen flexible polymeric extrusion assembly 420, secured at their distal ends at a single purse down point 356, envelop and retain target tissue volume 354 within tissue capture basket 326 so that the practitioner can withdraw the delivery cannula 22 along with its retained target tissue volume 354 from the healthy tissue 366 of the patient following the same path of insertion 363 as seen in FIG. 26.

Returning to FIG. 28, once the tissue capture assembly 329 forming a basket around the contained target tissue volume 354 is withdrawn, a scissors or other cutting device may be used to cut the resistively heated portion of cutting and pursing cable at the substantially single purse down point 356 thereby allowing one or more of the first through fifth leaf members 82-86 and the multi-lumen flexible polymeric extrusion members to open thereby releasing the captured target tissue volume 354 from the tissue cutting and capture assembly 329. Following the extraction of the target tissue volume 354 from the tissue cutting and capture assembly 329, the target tissue volume is placed in a specimen container, immersed in a volume of fixative agent such as 3.7% formaldehyde in water, the volume being at least ten times the volume of the specimen. The specimen remains in the fixative agent for a period preferably sufficient to ensure complete penetration of the target tissue volume 354. By way of example, the duration of immersion in fixative agent may be about five hours or more depending on the size of the target tissue volume, the duration of immersion in the fixative agent increasing with the size of target tissue volume 354. Following fixation of the target tissue volume 354 it is typically sectioned for subsequent histomorphologic diagnosis by a pathologist. In this regard, see Hewitt, S., et. al., Tissue Handling and Specimen Preservation in Surgical Pathology. Archives of Pathology & Laboratory Medicine 2008; 132: 1929-1935.

As seen in FIG. 27, the practitioner selectable position of the second pivotable drive finger 185b determines the extent of the enlargement of tissue capture basket 326. The extent of the enlargement of tissue capture basket 326 is also seen in FIG. 9 in the end view of the tissue capture basket 326 at the fourth and largest stage of enlargement of the first through fifth leaf members 82d-86d and the multi-lumen flexible polymeric extrusion member 420 that combine to form the first through sixth segments of the resistively heated portion of cutting and pursing cable 89-94.

The range of preferred dimensions for the various components seen in FIGS. 1A-25 are listed below where all dimensions are in units of inches unless specified otherwise and are labeled as shown in the referenced figures.

| | |
|---|---|
| L₁ = 8.0 to 12.0 | D₁ = 0.23 to 0.30 |
| L₂ = 5.0 to 7.0 | D₄ = 0.20 to 0.27 |
| L₃ = 3.5 to 6.5 | D₅ = D₂₁ = 0.007 to 0.011 |
| L₄ = 0.12 to 0.26 | D₆ = 0.010 to 0.014 |
| L₅ = 0.2 to 0.3 | D₇ = 0.70 to 0.85 |
| L₆ = 11.0 to 13.5 | D₈ = 0.090 to 0.120 |
| L₇ = 0.1 to 0.2 | D₉ = 0.070 to 0.090 |
| L₈ = 0.05 to 0.07 | D₁₀ = 15 mm to 30 mm |
| L₉ = 2.8 to 3.8 | D₁₁ = 0.25 to 0.33 |
| L₁₀ = 0.5 to 0.6 | D₁₂ = 0.4 to 0.5 |
| L₁₁ = 0.15 to 0.30 | D₁₃ = 0.7 to 0.9 |
| L₁₂ = 3.1 to 4.0 | D₁₄ = 1.25 to 1.3 |
| L₁₃ = 0.14 to 0.17 | D₁₅ = D_{wire} = 0.0010 to 0.0015 |
| L₁₄ = 0.25 to 0.35 | D₁₆ = 0.0030 to 0. 0085 |
| L₁₅ = 0.052 to 0.062 | D₁₇ = 0.60 to 0.75 |
| L₁₈ = 0.85 to 1.250 | D₁₈ = 0.19 to 0.25 |
| L₂₀ = 0.30 to 0.45 | D₁₉ = 0.50 to 0.65 |
| L₂₁ = 0.43 to 0.50 | D₂₀ = 15 to 30 mm |
| L₂₂ = 0.80 to 1.50 | |
| L₂₃ = 0.71 to 0.84 | |
| L₂₄ = 0.84 to 1.250 | Θ₁ = 50° to 90° |
| L₂₅ = 0.009 to 0.020 | Θ₂ = 35° to 50° |
| L₂₆ = 0.018 to 0.022 | H₁ = 1.5 to 2.5 |
| L₂₇ = 0.018 to 0.022 | H₂ = 0.020 to 0.035 |
| L₂₈ = 0.027 to 0.032 | H₃ = 0.010 to 0.017 |
| L₂₉ = 0.10 to 0.16 | H₄ = 0.040 to 0.070 |
| L₃₀ = 0.50 to 0.75 | H₅ = 0.75 to 1.00 |
| L₃₁ = 0.40 to 0.60 | H₆ = 0.045 to 0.065 |
| L₃₂ = 0.40 to 0.50 | H₇ = 0.060 to 0.120 |
| L₃₃ = 2.0 to 9.0 | H₈ = 0.10 to 0.15 |
| L₃₄ = 0.065 to 0.090 | H₉ = 0.010 to 0.018 |
| L₃₅ = 0.11 to 0.16 | W₈ = 0.075 to 0.095 |
| L₃₆ = 1.90 to 2.10 | W₁ = 1.0 to 1.5 |
| t₁ = 0.0030 to 0.0070 | W₂ = 0.23 to 0.30 |
| t₂ = 0.015 to 0.030 | W₃ = 0.020 to 0.035 |
| t₃ = 0.080 to 0.120 | W₄ = 0.060 to 0.090 |
| t₄ = 0.010 to 0.015 | W₅ = 0.065 to 0.095 |
| Φ₁ = 55° to 65° | W₆ = 0.085 to 0.115 |
| Φ₂ = 55° to 65° | W₇ = 0.090 to 0.125 |
| Φ₃ = 55° to 65° | W₉ = 0.90 to 0.140 |
| Φ₄ = 55° to 65° | W₁₀ = 0.175 to 0.200 |
| Φ₅ = 55° to 65° | W₁₁ = 0.028 to 0.033 |
| Φ₆ = 55° to 65° | W₁₂ = 0.019 to 0.022 |
| Δₜ = 0.001 to 0.003 second | W₁₃ = 0.18 to 0.21 |
| Δₘₐₓ = 0.3 to 0.5 ohms | W₁₄ = 0.020 to 0.040 |
| Δₚₐᵤₛₑ = 0.1 to 0.3 second | W₁₅ = 0.025 to 0.035 |
| K₁ = 0.05 to 0.20 pounds/inch | W₁₆ = 0.063 to 0.093 |

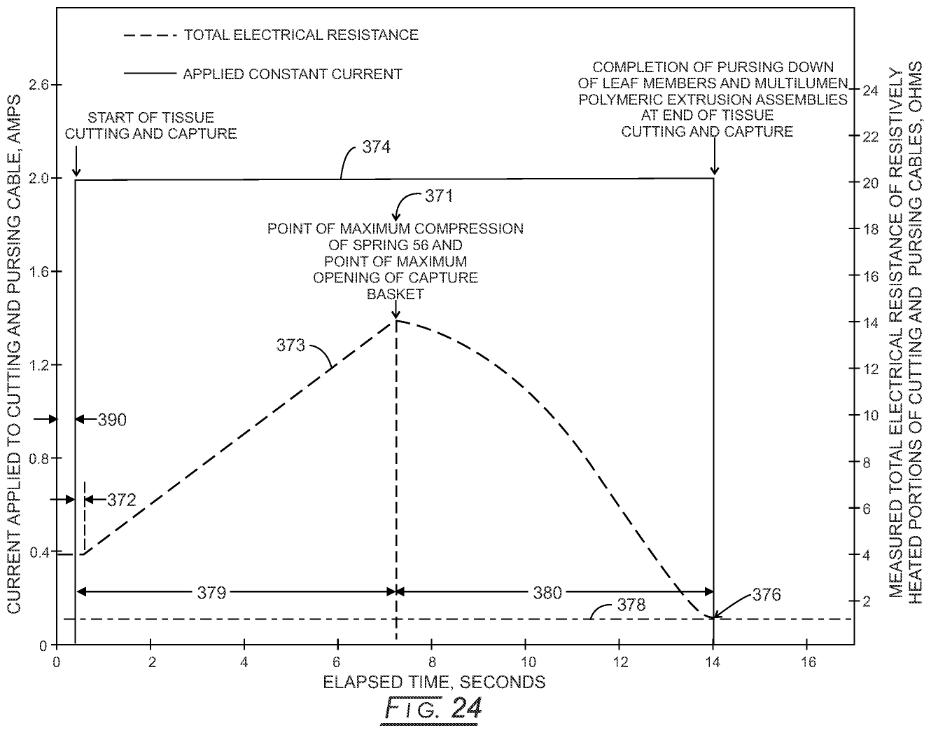

Turning now to FIGS. 1, 3, 24 and 26A, an example of the applied constant current level 374 and the total electrical resistance 373 of the combined first through sixth segments of the resistively heated portion of the cutting and pursing cable 89-94 is presented in graphical form as a function of elapsed time. Upon the manual depression of the start tissue incision and capture switch 39 by practitioner, the motor and planetary gear assembly 170a seen in FIG. 3 is briefly energized by an applied DC voltage (e.g., 6.0 volts applied for 300 milliseconds) resulting in the brief advancement of the translation nut 182a and first pivotable drive finger 185a and corresponding brief advancement of drive assembly drive member 324 as seen at 390 in FIG. 24. The brief advancement of drive assembly drive member 324 induces a corresponding advancement of leaf member and multi-lumen extrusion assembly 400 so that the first through sixth segments of the resistively heated portion of the cutting and pursing cable 89-94 are raised above the surface of the cannula distal end assembly 25 (e.g., by a distance of 0.030 to 0.040 inch) and in thermal contact with healthy tissue 366.

By way of a continuing example and referring to FIGS. 1, 3, 9A, 24, 26A, 27 and 28, the applied DC voltage to motor and planetary gear assembly 170a is briefly suspended and the constant current 374 is applied to the first through sixth segments of the resistively heated portion of the cutting and pursing cable 89-94 (e.g., at a predetermined level of 2.00 amps at 100 kHz corresponding to the diameter of cutting and pursing cable 33 and preferred heat flux level of 220 watts/cm²) while the resistively heated portions of cutting and pursing cables are stationary resulting in pre-heating period 372 as seen in FIG. 24. As seen in FIG. 24, the duration of the pre-heating period 372 may, for example, be 20 to 30 milliseconds. As seen in the example graph shown in FIG. 24, the pre-selected level of constant current 374 (e.g., 2.00 amps at 100 kHz) continues to be applied until the elapsed time at which the tissue cutting and capture ends 376 corresponding to the complete purse down of tissue capture basket 326 as seen graphically in FIG. 28 at, for example, an elapsed time of about 14 seconds and as also seen in cross-sectional view in FIG. 28. In the example graph of FIG. 24 and as seen in FIG. 27, during the period of the expansion 379 of the tissue capture basket 326, compression spring 56 compresses as cable mounting hub 296 advances along advancement path 403 from initial position A at 401 to its furthest point of advance A′ seen at position 402 corresponding to the maximum opening of the tissue capture basket 326. The compression spring 56 has a small spring force constant (e.g., 0.05 to 0.20 pounds per inch deflection) and maintains a sufficient level of tension in the cutting and pursing cable 33 to maintain a low level of sliding contact resistance between the cutting and pursing cable 33 and the first electrically and thermally conductive eyelet 446 as well as between the cutting and pursing cable 33 the second electrically and thermally conductive eyelets 450. At the point of maximum opening 371 of tissue capture basket 326, the first and second tensioning portions 118 and 119 of the cutting and pursing cable 33 become taught as the cable mounting hub 296 engages stationary second pivoting drive finger 185b and detectable within circuit board assembly 184 by a measurable increase in the electrical current level supplied to first motor 170a driving first motor-actuated drive tube drive member assembly thereby initiating the commencement of purse down of the tissue capture basket 326.

Once first and second tensioning portions 118 and 119 of the cutting and pursing cable 33 become constrained as the cable mounting hub 296 abuts the second pivoting drive finger 185b, secured to second transition nut 182b as part of second motor-actuated cable mounting hub translation assembly 180b, then the pursing down of the distal ends of the first through fifth leaf members 82-86 and the distal end of the multi-lumen flexible polymeric extrusion assembly 420 commences. Referring to FIGS. 3 and 24, during the pursing down period 380 and beginning at the point of maximum opening 371 of tissue capture basket 326, a predetermined level of voltage is applied to second motor 170b causing second motor-actuated cable mounting hub translation assembly 180b to advance second pivoting drive finger 185b in rearward direction 477 thereby increasing the rate of pursing down of the distal ends of the first through fifth leaf members 82-86 and the distal end of the multi-lumen flexible polymeric extrusion assembly 420.

During the period of expansion 379 of the tissue capture basket 326, as seen in FIG. 24, the measured total electrical resistance 373 of the [a] first through sixth segments of the resistively heated portion of the cutting and pursing cable 89-94 and [b] electrical contact resistances at the sliding interfaces between the resistively heated portion of the cutting pursing cable and the contacted surfaces of the first and second electrically and thermally conductive eyelets 446 and 450, respectively, increases from the start of tissue cutting and capture up to the point of maximum opening 371 of the tissue capture basket 326. At the point of maximum opening 371 of the tissue capture basket 326, the total length of the resistively heated portion of the cutting and pursing cable is at its maximum extent and, correspondingly, the total electrical resistance of the first through sixth segments of the resistively heated portion of the cutting and pursing cable 89-94 (including electrical contact resistances at first and second electrically and thermally conductive eyelets 446 and 450, respectively) is at a maximum level. Conversely, during the period of pursing down 380 of the distal ends of the first through fifth leaf members 82-86 and the distal end of the multi-lumen flexible polymeric extrusion assembly 426 or 427, the total length of the resistively heated portion of the cutting and pursing cable decreases with a corresponding decrease in the total electrical resistance 373.

The decrease of the total electrical resistance 373 to its minimum extent 378 corresponds to the completion of the pursing down of the tissue capture basket 326. The time at which the measured total resistance of the first through sixth segments of the resistively heated portion of the cutting and pursing cable 89-94 decreases to or less than a lower-limit total electric resistance level, R_{MIN} as seen at 378 (e.g. a value in the range from about 1.0 to 2.0 ohms) corresponds to the time at which tissue cutting and capture ends 376 as seen in FIG. 24. The time at which tissue cutting and capture ends 376 coincides with the suspension of applied constant current, applied voltage to motors 170a and 170b, the termination of the audible tone generated by speaker 200 within handpiece assembly 15 as well as the illumination of the capture “Complete” indicator light 52 as seen in FIG. 1. Following the illumination of the capture “Complete” indicator light 52, the delivery cannula 22 with the tissue cutting and capture assembly 329 as seen in FIG. 28 is withdrawn by the practitioner from the patient.

Turning now to FIGS. 22, 24 and 25, the upward slope of the total electrical resistance 373 during the period of the expansion phase 379 of the tissue capture basket 326 is determined by the angle of the leaf member deployment ramps 289a-289e and the equivalent angle of the multi-lumen flexible polymeric extrusion assembly deployment ramp 290 in tip component 266. The equivalent angles of the leaf member deployment ramps 289a-289e and the angle of the multi-lumen flexible polymeric extrusion assembly deployment ramp 290 in tip component 266 contributes to the determination of the angle of deployment, Θ₂ as seen in FIG. 25 at 345, for leaf members 82-86 and multi-lumen flexible polymeric extrusion assembly 426 or 427. The predetermined angle of deployment, Θ₂ of the leaf members and the multi-lumen flexible polymeric extrusion assembly, as seen at 345 in FIG. 25, correspondingly determines the rate at which total length of the first through sixth segments of the resistively heated portion of the cutting and pursing cable 89-94 increases as seen in FIG. 9 for four illustrated stages of deployment of the leaf members 82-86 and the multi-lumen flexible polymeric extrusion assembly 426 or 427 with corresponding increases in the first through sixth segments of the of the resistively heated portion of the cutting and pursing cable 89-94. If the leaf members 82-86 and the multi-lumen flexible polymeric extrusion assembly 426 or 427 deploy as intended and substantially along the predetermined angle of deployment, Θ₂, then the rate of increase of the measured total electrical resistance 373 will likewise follow a predetermined measured rate of resistance increase, Rᵣₐₜₑ as illustrated by positive slope 373 in FIG. 24. However, if a malfunction occurs during the deployment of the tissue capture basket 326 causing one of more leaf members 82-86 and/or multi-lumen flexible polymeric extrusion assembly 426 or 427 fail to deploy along the predetermined ramp, then the corresponding measured rate of total electrical resistance increase, Rᵣₐₜₑ will be less than a predetermined minimum rate of resistance increase, Rᵣₐₜₑₘᵢₙ. If the measured rate of total electrical resistance increase, Rᵣₐₜₑ is less than a predetermined minimum rate of resistance increase, Rᵣₐₜₑₘᵢₙ during the opening phase of the tissue capture basket 326, then programming within microcomputer 202 in the circuit board assembly 184 interrupts the deployment of the tissue capture basket 326 and begins flashing the “Capturing” indicator light 46, stops the application of constant current to the first through sixth segments of the resistively heated portion of the cutting and pursing cable 89-94, stops the application of voltage to first motor 170a and discontinues the audible tone generated by speaker 200 within handpiece assembly 15 to alert the practitioner that a malfunction has occurred.

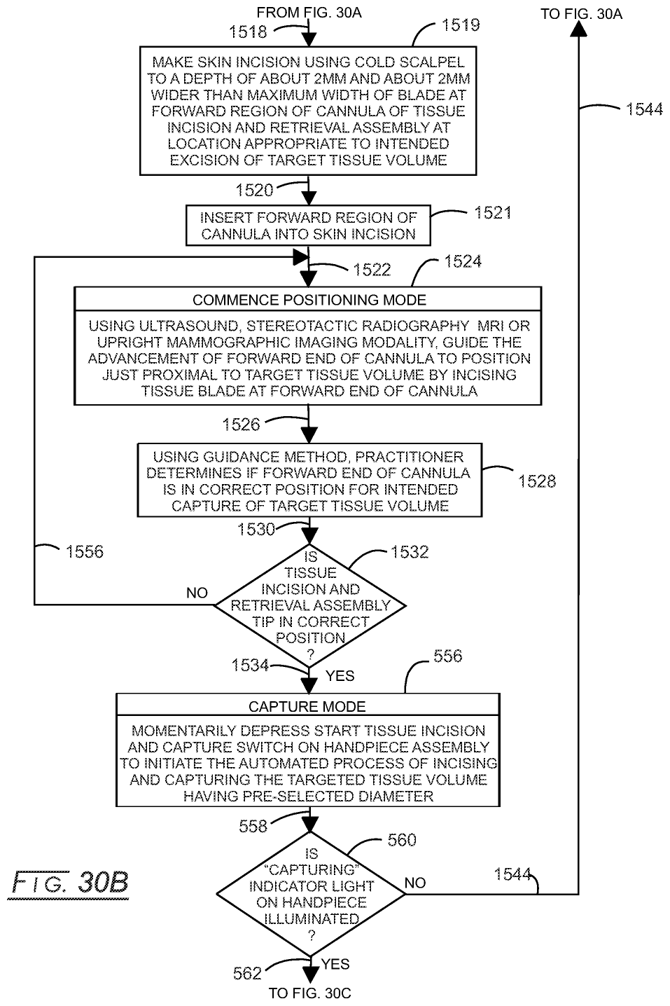

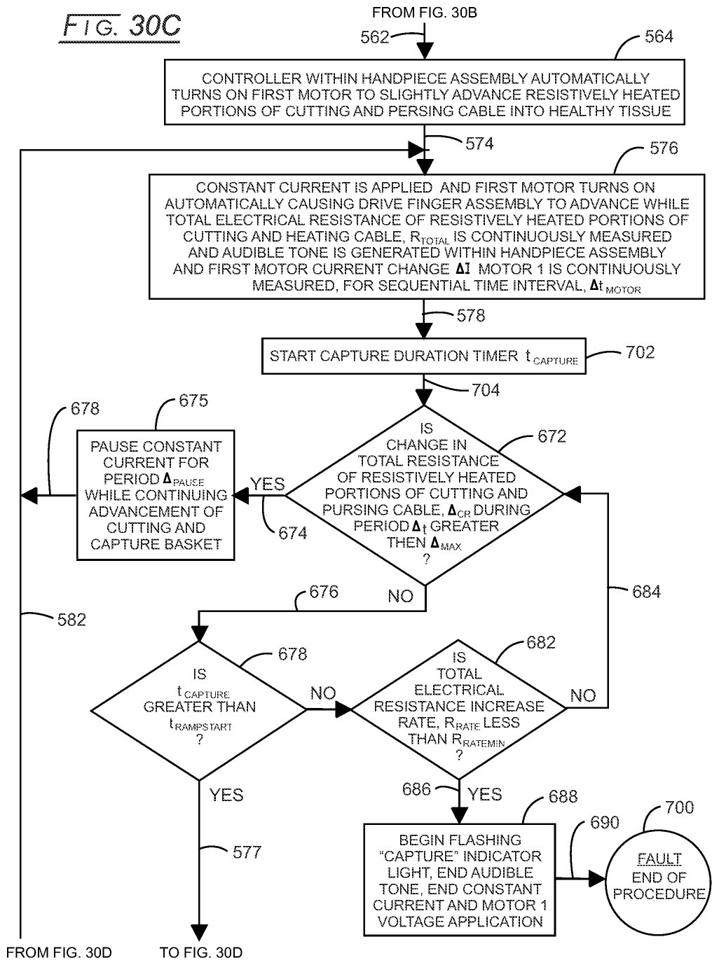

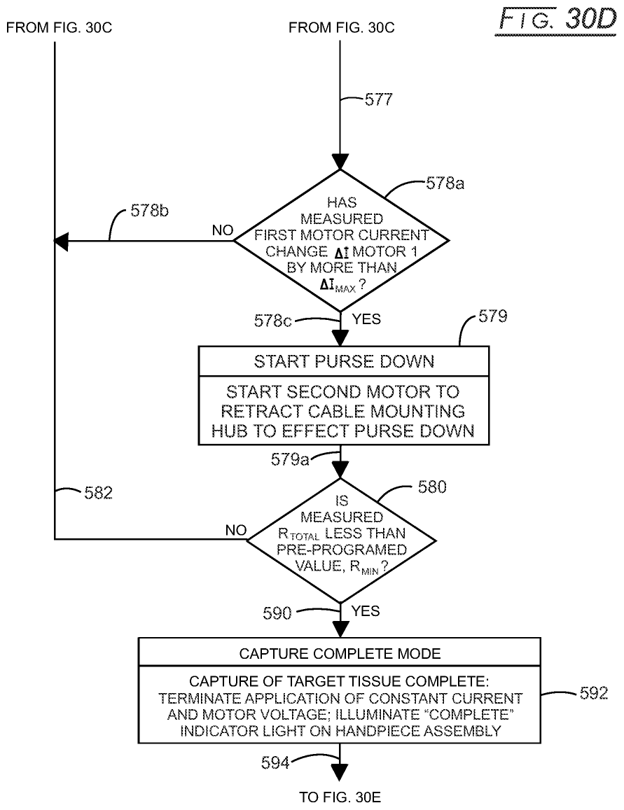

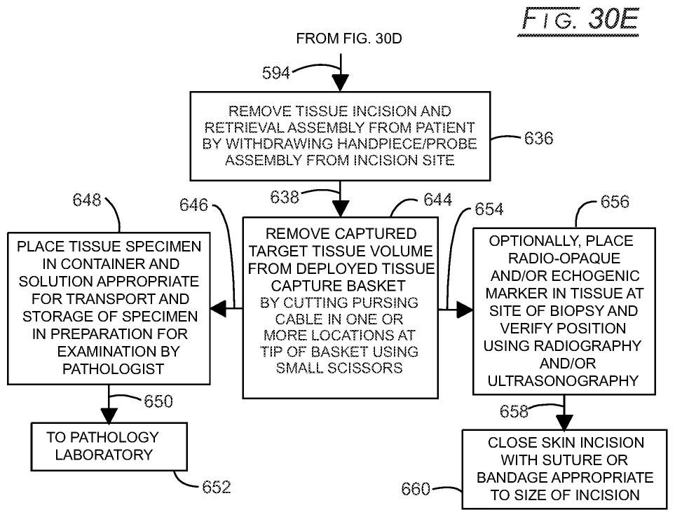

The set of FIGS. 30A-30E combine, as labeled thereon, to provide a flow chart describing the operation of the instant system. In the discourse to follow, the term tissue incision and retrieval system 10 is comprised of a single-use tissue incision and retrieval assembly 12 and reusable handpiece assembly 15 as seen in FIGS. 1, 1A and 2. The term “controller” refers to internal energy source and control system 181 comprising rechargeable battery 183 and circuit board assembly 184. By way of example, the circuit board assembly 184 comprises [a] microcomputer 202 and programmed logic to control tissue incision and capture functions performed by handpiece assembly 15 (not shown), [b] constant current source 247, [c] constant voltage sources (not shown) for energizing first motor 170a and second motor 170b, [d] first switch sensor 48, [e] second switch sensor 49, [f] first light emitting diode 44, [g] second light emitting diode 50, [h] third light emitting diode 54, [i] first constant current source connector pin 51, [j] second constant current source connector pin 53 and [k] capture size selection switch 479 as well as numerical capture size display 486. Cueing “Ready” indicator light 42, “Capturing” indicator light 46 and “Complete” indicator light 52 representing operational modes as well as on/off initialization switch 40 and start tissue incision/capture switch 39 are accessible on the top portion of handpiece assembly 15 as seen in FIG. 1.

Looking to FIGS. 1, 2, 8, 26-28 and 30A, preparation in advance of the procedure, as represented at block 1496, provides for the recharging of the internal battery within handpiece assembly 15 using handpiece assembly battery charger 9. At the start of a procedure, practitioner selects desired size (i.e., maximum diameter) of capture for intended target tissue volume 354 using capture size selection switch 479 on surface of reusable handpiece assembly 15 as represented at line 1498 and block 1500. Practitioner selection is made with respect to the predetermined size of the tissue volume to be removed. In general, the resistively heated portion of cutting and pursing cable, multi-lumen flexible polymeric extrusion assembly and leaf members will extend through healthy tissue 366 surrounding a targeted suspicious and potentially malignant lesion 368, as seen in FIGS. 26 through 28. By way of example, if the maximum dimensional extent of suspicious and potentially malignant lesion 368 is determined to be 10 mm (0.4 inch) based on prior examination using ultrasound and/or radiographic imaging, then practitioner may select a capture size of 25 mm (1.0 inch) to provide a boundary layer of healthy tissue 367 surrounding the suspicious and potentially malignant lesion 368 as seen in FIGS. 26, 26A, 27 and 28. This selection of a larger capture size avoids complications of spreading potentially malignant cells and the like upon removal of the suspicious and potentially malignant lesion 368 since the suspicious and potentially malignant lesion is surrounded by a boundary layer of healthy tissue 367. Also, the verification of the presence of a boundary layer of healthy tissue 367 around the entire periphery of the targeted suspicious and potentially malignant lesion 368, as determined at pathological examination of the excised tissue sample, represents the complete removal of the targeted suspicious and potentially malignant lesion 368 and may avoid the need for further surgical removal of tissue at the site of the targeted tissue volume 354.

Once practitioner selects the desired diameter D₁₀ of a substantially spherical volume of tissue, the practitioner inserts tissue incision and retrieval assembly 12 into handpiece assembly 15, as represented at line 1502 and block 1504 and may be enveloped within optional single-use, transparent and flexible sterile sheath 109, as represented at line 1552 and block 1504. Next, practitioner momentarily depresses on/off and initialization switch 40 on handpiece assembly 15 and visually confirms if the indicator light 42 (e.g., yellow light) adjacent to the label “Ready” on the handpiece assembly is illuminated, as represented at line 1506 and block 1508. Where the “Ready” indicator light 42 is not illuminated, the activity described at block 1512 fails and the procedure reverts as represented at line 1513 to line 1544 and block 1546, the practitioner having been pre-instructed that a non-functional handpiece assembly 15 is at hand and the procedure reverts to selecting another handpiece assembly 15 to replace the non-functional handpiece assembly 15, as represented at line 1548 and block 1550. Following the replacement of the non-functional handpiece assembly 15, practitioner inserts sterile tissue incision and retrieval assembly 12 into handpiece assembly 15 where the handpiece assembly may be enveloped within optional single-use, transparent and flexible sterile sheath 109, as represented at line 1552 and block 1504. Where the “Ready” indicator light 42 is illuminated, the activity described at block 1512 proceeds to the next step in the procedure.

Referring now to FIGS. 1A, 26, 26A, 30A and 30B, practitioner next administers local anesthetic agent (e.g., lidocaine) at skin level and within region of intended excision of target tissue volume 354, as represented at line 1514 and block 1516. For example, this step should be performed at least five minutes before the start of the biopsy procedure to assure perfusion of the tissue surrounding the target tissue volume 354 with the anesthetic agent. Waiting periods of at least five minutes also avoids the entrapment of a bolus of anesthetic liquid along the path of the tissue cutting and capture procedure.

Next, practitioner makes an incision at skin site 24 using a cold scalpel to a depth of 2 mm to 4 mm and a width about 2 mm wider than the maximum width of blade 31 located at forward end 27 of delivery cannula 22 (e.g., an incision width of about 8 mm) at the location appropriate to the intended excision of target tissue volume 354, as represented at line 1518 and block 1519. Practitioner next advances surgically sharp blade 31 at the forward end 27 of delivery cannula 22 into incision at skin site 24 to a depth of 2 mm to 4 mm, as represented at line 1520 and block 1521. The positioning of the forward end of the cannula 27 commences using an ultrasound, stereotactic radiography, MRI or upright mammographic imaging modality to guide the advancement of the forward end 27 of delivery cannula 22 to a position just proximal to the target tissue volume 354, incising healthy tissue 366 using blade 31 at tip of tissue incision and retrieval assembly 12, as seen in FIG. 26A and as represented at line 1522 and block 1524.

Referring now to FIGS. 1A, 26A and 30B, practitioner determines, using the guidance method, if forward end 27 of delivery cannula 22 is in the correct position for the intended capture of target tissue volume 354, as represented at line 1526 and block 1528 followed by Line 1530 and block 1532. If the forward end 27 of delivery cannula 22 is not in the correct position for the intended capture of target tissue volume 354, practitioner uses the guidance method to re-position forward end of the cannula 27 to correct position just proximal to the target tissue volume 354, as represented at line 1556 and returning to line 1522 and block 1524. If the forward end 27 of delivery cannula 22 is in the correct position for the intended capture of target tissue volume 354, the procedure proceeds to the next step. In the next step, as seen in FIGS. 27 and 28, target tissue volume 354 is incised from healthy tissue 366 by the first through sixth segments of the resistively heated portion of the cutting and pursing cable 89-94, respectively, and circumscribed and contained within tissue capture basket 326.

Referring now to FIGS. 1A, 25, 26A, 27, 28 and 30B, practitioner momentarily depresses start tissue incision and capture switch 39 on handpiece assembly 15 to initiate the automated process of incising and capturing target tissue volume 354 having a selected diameter, D₁₀, the diameter pre-selected by practitioner (see block 1500), as represented at line 1534 and block 556. Capturing Indicator light 46 (e.g., green light) next to “Capturing” label on handpiece assembly 15 should be illuminated confirming the start of the capturing step in the procedure. Where the “Capturing” indicator light 46 is not illuminated due to a malfunction within the handpiece assembly 15, the activity described at block 560 fails and the procedure reverts as represented at line 1544 and block 1546, the practitioner having been pre-instructed that a non-functional handpiece assembly 15 is at hand and the procedure reverts to selecting another handpiece assembly 15 to replace the non-functional handpiece assembly 15, as represented at line 1548 and block 1550. Following the replacement of the non-functional handpiece assembly 15, practitioner re-inserts sterile tissue incision and retrieval assembly 12 into handpiece assembly 15 where the handpiece assembly is optionally enveloped within single-use, transparent and flexible sterile sheath 109, as represented at line 1552 and block 1504.

Referring now to FIGS. 1A, 3, 4, 26A, 27, 28 and 30C, where the “Capturing” indicator light 46 is illuminated, as represented at line 562 and block 564, the entry into the tissue capture mode starts a two-phase automated sequence. At phase one, motor 170a within handpiece assembly 15 is briefly energized by the application of the predetermined voltage level (e.g., 6.0 volts DC) for a predetermined period (e.g., about 0.3 second) as described in connection with FIGS. 3, 4 and 26A. This brief energizing of motor 170a advances motor-actuated drive tube drive member translation assembly 180a with a corresponding brief advancement of the distal ends of first through fifth leaf members 82-86 and distal end of multi-lumen flexible polymeric extrusion members 420. Brief advancement of distal ends of the leaf members and distal end multi-lumen flexible polymeric extrusion members assures that the supported first through sixth segments of the resistively heated portion of the cutting and pursing cable, 89-94, respectively, are advanced a short distance (e.g., 0.050″) into adjacent healthy tissue 366 as seen in FIG. 26A, as represented in block 564.

At phase two, still referring to FIGS. 1A, 3, 4, 26A, 27, 28 and 30C, internal energy source and control system 181 in handpiece assembly 15 applies a predetermined level of voltage to motor 170a. Simultaneously, a fixed, predetermined level of constant current (e.g., a fixed constant current level in the range from 1.2 to 2.5 amps at a frequency of 100 kHz) is applied to first through sixth segments of the resistively heated portion of the cutting and pursing cable, 89-94, respectively, by internal energy source 247 and control system 181 in handpiece assembly 15, thereby commencing the start of the tissue cutting and capture of the target tissue volume 354. Throughout the period during which constant current is applied to the first through sixth segments of the resistively heated portion of the cutting and pursing cable, 89-94, respectively, the total series resistance, R_{Total} of the first through sixth segments of the resistively heated portion of the cutting and pursing cable, 89-94, respectively, is continuously monitored. Also, throughout the period during which constant current is applied to the first through sixth segments of the resistively heated portion of the cutting and pursing cable, 89-94, respectively, an audible tone is generated within handpiece assembly 15 to provide a cue to the practitioner that the tissue cutting and capture process is continuing, as represented at line 574 and block 576 in FIG. 30C.

Programming within microcomputer 202 of circuit board assembly 184 next starts clock within microcomputer to accumulate capture duration time, t_{capture} during the initial deployment of the leaf members 82-86 and multi-lumen flexible extrusion assembly 426 or 427 toward the time at which the maximum opening of the tissue capture assembly occurs 371 as seen in FIG. 24 as represented at line 578 and block 702. The capture duration time, t_{capture} is initially set to 0.0 seconds at the time of initialization of procedure as earlier seen in block 508. Microcomputer 202 of circuit board assembly 184 also calculates a value for the maximum capture duration time limit, tᵣₐₘₚₛₜₐᵣₜ during which the time-based rate of increase in the electrical impedance of the resistively heated cutting and pursing cable circuit, Rᵣₐₜₑ is compared with a minimum rate of increase of the electrical impedance of the resistively heated portions of the cutting and pursing cable circuit, Rᵣₐₜₑₘᵢₙ. By way of example, the calculated value for the maximum capture duration time limit, tᵣₐₘₚₛₜₐᵣₜ may be 4, 5, 6 or 7 seconds for practitioner selected diameters of target tissue volumes 354 of 15, 20, 25 or 30 mm, respectively. The maximum capture duration time, tᵣₐₘₚₛₜₐᵣₜ is selected to occur prior to the start of curvature of the tissue capture basket 326 as seen in FIG. 25.

Programming within microcomputer 202 of circuit board assembly 184 next begins comparing the amount of increase of the continuously measured electrical impedance of the resistively heated cutting and pursing cable circuit, Δ_{CR} during a predetermined brief time interval, Δₜ with a pre-programmed maximum acceptable increase during the brief time step, Δₜ of the continuously measured electrical impedance of the resistively heated cutting and pursing cable circuit, Δₘₐₓ. In a preferred embodiment, the brief time interval, Δₜ is in the range from 0.001 to 0.003 second and the maximum acceptable increase of the continuously measured electrical impedance of the resistively heated cutting and pursing cable circuit, Δₘₐₓ is in the range from 0.3 to 0.5 ohm. As seen at line 704 and block 672, if a measured increase in the electrical impedance of the resistively heated cutting and pursing cable circuit, Δ_{CR} during a predetermined brief time interval, Δₜ exceeds the maximum acceptable increase, Δₘₐₓ, then the increase is attributable to a sudden loss of good electrical contact between first and/or sixth segments of the resistively heated portion of the cutting and pursing cable 89 and/or 94, respectively, and the first and/or second electrically and thermally conductive eyelets 446 and/or 450, respectively, (i.e., a sudden increase in the electrical contact resistance between the resistively heated cutting and pursing cable and the first and/or second electrically and thermally conductive eyelets). Upon the detection of a sudden loss of good electrical contact between the resistively heated cutting and pursing cable and the first and/or second electrically and thermally conductive eyelets corresponding to the measured value of Δ_{CR} exceeding Δₘₐₓ during a brief time interval, Δₜ, as seen at block 672, programming within microcomputer 202 in handpiece assembly 15 temporarily interrupts the supply of constant current for a predetermined time interval, Δₚₐᵤₛₑ while continuing to advance the multi-lumen flexible polymeric extrusion and leaf members to re-establish good electrical contact between the resistively heated cutting and pursing cable and the first and/or second electrically and thermally conductive eyelets as seen at line 674 and block 675. In a preferred embodiment, the duration of the interruption, Δₚₐᵤₛₑ ranges from 0.10 to 0.30 second. A momentary loss of acceptable electrical contact between the first and/or sixth segments of the resistively heated portion of the cutting and pursing cable 89 and/or 94, respectively, and the first and/or second electrically and thermally conductive eyelets 446 and/or 450, respectively, can result in an associated unacceptably large increase in the electrical contact resistance at the first transition boundary 396 and/or the second transition boundary 406, respectively, thereby causing overheating of the resistively heated cutting and pursing cable and its failure through melting or a significant decrease in the load-bearing capability to withstand the tensile forces applied to the resistively heated cutting and pursing cable.

As a result of the intentional interruption of the supply of constant current to the resistively heated cutting and pursing cable circuit upon the detection of an unacceptably large increase in the measured electrical impedance within the resistively heated cutting and pursing cable circuit, the resulting high level of Joulean heating at the interface the resistively heated cutting and pursing cable and the first and/or second electrically and thermally conductive eyelets and an associated significant increase in the localized temperature of the resistively heated cutting and pursing cable is avoided. The amount of localized Joulean heating (in watts) associated with unacceptably large electrical contact resistance at the interface between the first and sixth segments of the resistively heated portion of the cutting and pursing cable 89 and 94, respectively and the first and/or second electrically and thermally conductive eyelets 446 and 450, respectively, is equal to the product of the square of the applied constant current (in amps) and the unacceptably large value of the electrical contact resistance (in ohms) at the interface between the resistively heated cutting and pursing cable and the first and/or second electrically and thermally conductive eyelets. Upon the interruption of the supply of constant current for a brief period Δₚₐᵤₛₑ, the application of the constant current is resumed as seen in line 677 continuing to line 582 and returning to block 576 as seen in FIG. 30C.

As seen in block 672, if a measured increase in the electrical impedance of the resistively heated cutting and pursing cable circuit, Δ_{CR} during a predetermined brief time interval, Δₜ does not exceed the maximum acceptable increase, Δₘₐₓ, then the application of the predetermined constant current to the first through sixth segments of the resistively heated portion of the cutting and pursing cable, 89-94 as well as the predetermined constant voltage applied to first motor 170a continues to block 678 as seen at line 676.

As seen in block 678, if the accumulated capture duration time, t_{capture} is less than the computed maximum capture duration time limit, tᵣₐₘₚₛₜₐᵣₜ, then programming within microcomputer 202 of circuit board assembly 184 continues to block 682 at line 680 and compares the computed time-based electrical resistance increase rate, Rᵣₐₜₑ of the electrical impedance of the resistively heated cutting and pursing cable circuit with a predetermined minimum rate electrical resistance increase rate, Rᵣₐₜₑₘᵢₙ to determine if Rᵣₐₜₑ is less than Rᵣₐₜₑₘᵢₙ. In a preferred embodiment, the computed maximum capture duration time limit, tᵣₐₘₚₛₜₐᵣₜ of 5.0 seconds at an advancement speed of the first motor and planetary geartrain assembly 170a is at least 2.3 mm/second. Alternatively, if the advancement speed of the first motor and planetary geartrain assembly 170a is less than 2.3 mm/second, then the computed maximum capture duration time limit, tᵣₐₘₚₛₜₐᵣₜ is proportionately greater. By way of example, if the advancement speed of the first motor and planetary geartrain assembly 170a is 1.8 mm/second, then the computed maximum capture duration time limit, tᵣₐₘₚₛₜₐᵣₜ IS (2.3/1.8)×5.0 seconds or 6.4 seconds.

If Rᵣₐₜₑ is less than Rᵣₐₜₑₘᵢₙ, thereby representing a fault condition in the initial ramp of one or more leaf members 82-86 and/or the multi-lumen flexible polymeric extrusion assembly 426 or 427, the programming within microcomputer 202 of circuit board assembly 184 initiates the flashing of the “Capturing” indicator light 46 on handpiece assembly 15, stops the application of the predetermined level of constant current to the first through sixth segments of the resistively heated portion of the the cutting and capture cable 89-94, stops the application of the predetermined constant voltage applied to first motor 170a and stops the audible tone being issued by speaker 200 on circuit board assembly 184 as seen at line 686 and block 688 and represents the end of the procedure as seen at line 690 and block 700.

If Rᵣₐₜₑ is equal to or greater than Rᵣₐₜₑₘᵢₙ, thereby representing a normal condition in the initial ramp of one or more leaf members 82-86 and/or the multi-lumen flexible polymeric extrusion assembly 426 or 427, then programming within microcomputer 202 of circuit board assembly 184 returns to comparing the change in the total resistance, Δ_{CR} of resistively heated portions of cutting and pursing cable circuit during brief time period Δₜ with Δₘₐₓ as seen in line 684 and block 672.

As seen in block 678, if the accumulated capture duration time, t_{capture} is equal to or greater than the computed maximum capture duration time limit, tᵣₐₘₚₛₜₐᵣₜ, then programming within microcomputer 202 of circuit board assembly 184 continues to block 578a as seen at line 577. If the change, ΔI in the continuously measured current level delivered to first motor 170a during a brief time interval (e.g., 0.1 to 0.3 second) is less than a predetermined limit, ΔIₘₐₓ as seen in block 578a, then opening of tissue capture basket continues as seen at line 578b and block 576.

If the change, ΔI in the continuously measured current level delivered to first motor 170a during a brief time interval (e.g., 0.1 to 0.3 second) is equal to or greater than a predetermined limit, ΔIₘₐₓ as seen at line 577 and block 578a, then the purse down of tissue capture basket 326 commences as seen at line 578c and block 579. The purse down of tissue capture basket 326 commences upon the application of a predetermined voltage to second motor 170b as seen at line 578c and block 579. Beginning at the point of maximum opening 371 of tissue capture basket 326, pursing down of tissue capture basket 326 commences with the application of a predetermined level of voltage to second motor 170b causing second motor-actuated cable mounting hub translation assembly 180b to advance second pivoting drive finger 185b to be advanced in rearward direction 477 thereby increasing the rate of pursing down of the distal ends of the first through fifth leaf members 82-86 and the distal end of the multi-lumen flexible polymeric extrusion assembly 420.

Still referring to FIGS. 1A, 3, 4, 26A, 27, 28 and 30D, a predetermined voltage continues to be applied to motor 170a, a predetermined voltage continues to be applied to motor 170b and a predetermined level of constant current continues to be applied to the first through sixth segments of the resistively heated portion of the cutting and pursing cable, 89-94 until the pursing down of the distal ends of first through fifth leaf members 82-86 and the multi-lumen flexible polymeric extrusion assembly 426 is complete. During this interval of time, the total series resistance, R_{Total} of the first through sixth segments of the resistively heated portion of the cutting and pursing cable, 89-94, respectively, is continuously measured and compared with a pre-programmed minimum total resistance value, R_{Min} (e.g., 1.0 to 2.0 ohms) by internal energy source and control system 181 within handpiece assembly 15, as represented at line 579a and 580. If the measured total series resistance, R_{Total} is not less than the pre-programmed minimum total resistance value, R_{Min}, then the application of a predetermined voltage to motor 170a and the application of a predetermined voltage to motor 170b with the simultaneous application of a predetermined level of constant current to the first through sixth segments of the resistively heated portion of the cutting and pursing cable, 89-94, respectively, continues as represented at line 582 to line 574 and block 576.

Still referring to FIGS. 1A, 3, 4, 26A, 27, 28 and 30D, if the measured total series resistance, R_{Total} is less than the pre-programmed minimum total resistance value, R_{Min}, (see query in block 580) then the cutting of healthy tissue 366 by first through sixth segments of the resistively heated portion of the cutting and pursing cable, 89-94, respectively, and formation of a tissue capture basket 326 that circumscribes target tissue volume 354 is determined to be complete, as represented at Line 590 and block 592. Also, the point in time that the measured total series resistance, R_{Total} is less than the pre-programmed minimum total resistance value, R_{Min}, corresponds to the point in time that the distal ends of first through fifth leaf members 82-86, and distal end of multi-lumen flexible polymeric extrusion member 426 reach a substantially single pursed down point 356 as seen in FIG. 28. At this point in time, the capture complete indicator light 52 on handpiece assembly 15 is also illuminated adjacent to label “Complete”. Also, at this point in time, the application of the predetermined voltage to motor 170a the application of the predetermined voltage to motor 170b as well as the simultaneous application of a predetermined level of constant current to the first through sixth segments of the resistively heated portion of the cutting and pursing cable, 89-94 are terminated, as represented at line 590 and 592.

Referring now to FIGS. 1A, 26, 28 and 30E, upon an affirmative determination that tissue cutting and capture assembly 329 has completely circumscribed target tissue volume 354, practitioner removes the delivery cannula 22 along with the tissue capture basket 326 containing the target tissue volume 354, following the original path of insertion 363 to exit the healthy tissue 366 of patient at incision site 24, as represented at line 594 and block 636. During this removal, some stretching of the tissue and skin at incision site 24 typically will be encountered with little or no disfigurement ensuing.

Referring 1A, 9A, 16, 28 and 30E, as represented at line 638 and block 644, the captured target tissue volume 354 (i.e., tissue specimen) containing suspicious and potentially malignant lesion 368 is next removed from tissue capture basket 326 by severing the cutting and pursing cable 33 adjacent to an eyelet 327 at the distal end of one or more first through fifth leaf members 82-86 by way of example using a small scissors (e.g., tenotomy scissors). Place extracted target tissue volume 354 in a container with immersion in a fixative solution (e.g., fixative agent such as 3.7% formaldehyde in water) in preparation for subsequent diagnostic examination by a pathologist, as represented at line 646 and block 648. Next, target tissue volume 354 submerged in fixative solution with the container is transported to pathology laboratory, as represented at line 650 and block 652.

An optional arrangement is represented at line 654 and block 656. The latter block provides for placing a radio-opaque and/or echogenic marker in the tissue at the site from which the target tissue volume 354 is removed and verifying the position thereof using radiography or ultrasonography. If a radio-opaque and/or echogenic marker in not placed in the tissue at the site from which the target tissue volume 354 is removed, then proceed at line 658 to block 660.

Then, as represented at line 658 and block 660, the incision at skin site 24 is closed using appropriate conventional closure techniques.

Referring now to FIG. 31, a photograph of a side view of a captured target tissue volume 354 following the completion of the deployment of the tissue cutting and capture assembly 329 that emerges from the cannula distal end assembly 25 disposed at distal end of delivery cannula 22. As seen in FIG. 28, multi-lumen flexible polymeric extrusion assembly 427 in combination with leaf members 82-86 (only leaf members 82 and 83 visible in FIG. 31) configured in a hexagonal shaped pattern of the resistively heated cutting and pursing cable segments form a complete circumscribing cutting and pursing cable path as the multi-lumen flexible polymeric extrusion assembly 427 in combination with leaf members 82-86 are advanced through the tissue being incised by the resistively heated portion of the cutting and pursing cable (not shown) and reach a substantially single pursed down point 356 as also seen in FIG. 28.

Since certain changes may be made in the above method, system and apparatus without departing from the scope of the present disclosure herein involved, it is intended that all matter contained in the above description or shown in the accompanying drawings shall be interpreted as illustrative and not in a limiting sense. For example, throughout the disclosure presented herein, the first through sixth segments of the resistively heated portion of the cutting and pursing cables 89-94, respectively, containing multiple wires as seen in FIG. 9A could be replaced by resistively heated portions of cutting and pursing wires, i.e., comprising a single wire of electrically conductive metal (e.g., titanium or titanium alloy wire).

While the apparatus, system, and method have been described with reference to various embodiments, those skilled in the art will understand that various changes may be made and equivalents may be substituted for elements thereof without departing from the scope and essence of the disclosure. In addition, many modifications may be made to adapt a particular situation or material in accordance with the teachings of the disclosure without departing from the essential scope thereof. Therefore, it is intended that the disclosure not be limited to the particular embodiments disclosed, but that the disclosure will include all embodiments falling within the scope of the appended claims. In this application all units are in the metric system and all amounts and percentages are by weight, unless otherwise expressly indicated. Also, all citations referred herein are expressly incorporated herein by reference.

1. Apparatus for retrieving a targeted tissue volume (354) of predetermined peripheral extent, comprising:
(a) a handpiece assembly (15) incorporating a circuit board assembly (184), a rechargeable battery (183), a constant current source (247), user-accessible control switches (39, 40), visible indicators (42, 46, 50), a speaker (200), a user-accessible capture size selection switch (479) located on the handpiece assembly for pre-selection of a diameter of a substantially spherical volume of tissue, and a display of selected capture size (485);
(b) a first motor and motor-actuated drive tube drive member translation assembly (180a) located within the handpiece assembly;
(c) a second motor and motor-actuated cable mounting hub translation assembly (180b) located within the handpiece assembly;
(d) a first pivotable drive finger (185a) drivingly engageable with a drive assembly drive member (324), the drive assembly drive member driven forwardly by the first motor and motor-actuated drive tube drive member translation assembly;
(e) a second pivotable drive finger (185b) drivingly engageable with a cable mounting hub (296), the second drive finger driven rearwardly by the second motor and motor-actuated cable mounting hub translation assembly;
(f) a programmed first microcomputer (202a) within the circuit board assembly (184) that responds to signals from user-accessible control switches (39, 40), the user-accessible control switches for controlling illumination of the visible indicators (42, 46, 50), for controlling activation of an audible tone from the speaker (200) during period of application of constant current to a resistively heated portion of a cutting and pursing cable having first through sixth segments, for continuously measuring an electrical impedance of the resistively heated portion of the cutting and pursing cable circuit during the application of constant current by the constant current source to the first through sixth segments of the resistively heated portions (89-94) of the cutting and pursing cable;
(g) a multi-lumen flexible polymeric extrusion and leaf members having a distal end;
(h) first and second electrically and thermally conductive eyelets (533a, 533b) located at the distal end of the multi-lumen flexible polymeric extrusion assembly (427);
(i) a programmed second microcomputer (202b) within the circuit board assembly that:
(1) interrupts the application of constant current by the constant current source to the resistively heated cutting and pursing cable circuit for a period of about 100 to 200 milliseconds while the second motor and motor-actuated cable mounting hub translation assembly continues to advance the multi-lumen flexible polymeric extrusion and leaf members for a time period Δₚₐᵤₛₑ if the measured increase in the electrical impedance of the resistively heated cutting and pursing cable circuit, Δ_{CR} during the predetermined time interval, Δₜ exceeds the maximum acceptable increase, Δₘₐₓ of about 0.5 to 1.0 ohms that can be attributed to electrical contact between the first segment (89), the sixth segment (94), or both of the first and sixth segments (89 and 94) of the resistively heated portion of the cutting and pursing cable and the first second electrically and thermally conductive eyelet (446), the second electrically and thermally conductive eyelet (450), or both the first and second electrically and thermally conductive eyelets (446 and 450), respectively;
(2) computes the rate of increase, Rᵣₐₜₑ of electrical impedance of the resistively heated cutting and pursing cable circuit during an initial time period of deployment, tᵣₐₘₚₛₜₐᵣₜ of the resistively heated cutting and pursing cable circuit corresponding to an initial deployment of the multi-lumen flexible polymeric extrusion and leaf members, which deployed multi-lumen flexible polymeric extrusion and leaf members form the tissue capture basket (326); and
(3) interrupts application of constant current and voltage supplied to the first motor and motor-actuated drive tube drive member translation assembly if the rate Rᵣₐₜₑ is less that a predetermined minimum rate of electrical resistance increase, Rᵣₐₜₑₘᵢₙ;
(j) a delivery cannula (22) having an outer surface surmounting an interior channel (263) and extending from a proximal end portion along a longitudinal axis (8) to a forward region (27) having a distal end assembly (25) positionable in confronting adjacency with the targeted tissue volume;
(k) a leaf member and multi-lumen flexible polymeric extrusion member assembly (400) positioned within the interior channel of the delivery cannula at the forward region of the delivery cannula having a forward region extending to forwardly disposed first through sixth segments of the resistively heated portion of the cutting and pursing cable (89-94) wherein a passage of electrical current is only through the first through sixth segments of the resistively heated portion of cutting and pursing cable to define a leading edge of the resistively heated portions of the cutting and pursing cable, a first tensionable portion (118) of the cutting and pursing cable extending proximally from the first segment of the resistively heated portion of cutting and pursing cable (89) into a third lumen within the multi-lumen flexible polymeric extrusion member and a second tensionable portion (119) of the cutting and pursing cable extending proximally from the sixth segment of the resistively heated portion of cutting and pursing cable (94) into a fourth lumen within the multi-lumen flexible polymeric extrusion member, the leading edge of the forward portion of the resistively heated portion of the cutting and pursing cable being extendable from the delivery cannula laterally outwardly and forwardly toward an outer peripheral dimension of the deployed tissue capture basket (326) having a predetermined maximum peripheral diametric extent and having a substantially circular cross-sectional shape effective to provide a circumspective positioning about the targeted tissue volume and, subsequently, the leading edge of the forward portion of the first through sixth segments of the resistively heated portion of the cutting and pursing cable (89-94) extendable while a mechanical load required for pursing down of the deployed tissue capture basket is applied to the first and the second tensionable portions of the cutting and pursing cable (118, 119) to a capture orientation enveloping the targeted tissue volume;
(l) a single-use support housing (100) having forward and rearward portions and coupled in supporting relationship with the delivery cannula at the proximal end portion of the delivery cannula;
(m) a tissue cutting and capture assembly (329) extending from driving engagement with a drive assembly drive member (324) to a driven engagement in the handpiece assembly (15) and drivably movable along the delivery cannula longitudinal axis from an initial position (391) to a final position of the leaf member and multi-lumen flexible polymeric extrusion assembly (400) and located at a tissue cutting and capture assembly position (394) wherein the initial position and final position are defined by an eyelet (327) carried at the distal ends of a plurality of leaf members (348) as well as at first and second electrically and thermally conductive eyelets (533a, 533b) at the distal end of multi-lumen flexible polymeric extrusion assembly (427); and
(n) a terminal assembly comprising first and second electrical contacts (120, 122) disposed on the exterior of the single-use support housing (100) oriented to make electrical contact with corresponding first and second electrical terminals (186, 188), respectively, disposed within the handpiece assembly that selectively receive a controlled level of constant current at an elevated frequency, the constant current being applied to the resistively heated portions of the cutting and pursing cable (89-94), commencing when tissue cutting and capture assembly (329) is just proximal and adjacent to the target tissue volume (354) of the eyelet containing tip (330) of each of the leaf members (348) as well as first and second eyelets (533a, 533b) at the distal ends of first and second electrically and thermally conductive lead wires (518, 520), respectively, disposed at the distal end of multi-lumen flexible polymeric extrusion assembly (427), continuing with position of tissue cutting and capture assembly (329) defined by the position of eyelets (327a-327e) of leaf members (82-86) as well as the position of first and second eyelets (533a,533b) at the distal end of multi-lumen flexible polymeric extrusion (427) are at a maximum opening of the tissue capture assembly (392) and continuing further until the first through sixth segments of the resistively heated portion of the cutting and pursing cable (89-94) purse down at the end of tissue cutting and capture (394).

2. The apparatus of claim 1, wherein the proximal ends of first and second tensionable portions of the cutting and pursing cables (118, 119) are secured to cable mounting hub (296), the cable mounting hub being drivingly movable by the first and second tensionable portions of cutting and pursing cables along the longitudinal axis (8) in correspondence with the advancement of the drive assembly drive member (324) from an initial position (401) of cable mounting hub to position (402) of cable mounting hub to define a partially deployed tissue capture basket (326) at a position (392) representing the maximum opening of the tissue cutting and capture assembly (329); and
corresponding to detectable engagement of cable mounting hub with second pivoting drive finger (185b) and to effect the subsequent pursing down of the deployed tissue capture basket (326) by a second motor-actuated cable mounting hub translation assembly (180b) driven in the second movement direction (405) of the cable mounting hub, thereby applying a mechanical load or tension to the first and second tensionable portions of the cutting and pursing cables (118, 119).

3. The apparatus of claim 1, wherein the first through sixth segments of the resistively heated portion of electrically conductive cutting and pursing cable (89-94) is a single metal wire.

4. The apparatus of claim 1, including a sharp cutting blade assembly disposed at the forward end (27) of the delivery cannula (22) wherein tip of blade (31) is coincident with the longitudinal axis (8) and enables an initial positioning of cannula distal end (25) in a confronting adjacency with respect to a targeted tissue volume (354).

5. The apparatus of claim 1, wherein the cutting and pursing cable (33) comprises a multiplicity of stainless steel wires, each wire having a diameter of between about 0.0008 to about 0.002 inch.

6. The apparatus of claim 3, wherein the metal wire is stainless steel, nickel, nickel alloy, titanium or titanium alloy having a diameter between about 0.002 to about 0.005 inch.

7. The apparatus of claim 1, further comprising:
an elongate support tube (282) extending within the delivery cannula along the longitudinal axis from the forward end (27) of delivery cannula (22) into a single-use support housing (100) and secured thereto adjacent the rearward portion of disposable support housing (100), wherein drive assembly drive member (324) is positioned over the support tube and the disposable support housing is located within the handpiece assembly (15) having a first pivotable drive finger (185a) and drivingly engagable with the drive assembly drive member (324) and engagable with a first motor-actuated drive tube drive member translation assembly (180a) to move tissue cutting and capture assembly (329) from the initial position (391) of eyelet containing tip (330) of leaf members (82-86) and eyelet containing tip of multi-lumen flexible polymeric extrusion member (427) to tissue cutting and capture assembly position (394) at the end of tissue cutting and capture.

8. The apparatus of claim 2, wherein second pivoting drive finger (185b) attached to second motor-actuated cable mounting hub translation assembly (180b) functions as a stop, the second pivoting drive finger (185b) prepositioned by practitioner selection of maximum diametric extent of tissue cutting and capture assembly (329) using capture size selection switch (479) on handpiece assembly.

9. The apparatus of claim 1, wherein functions of first and second microcomputers (202a, 202b) may be combined into a single microcomputer (202).

10. The apparatus of claim 1, wherein the elevated frequency of the constant current source is 25 kHz or greater.

11. The apparatus of claim 1, wherein the tissue cutting and capture assembly (329) comprises:
a plurality of leaf members (82-86) and a multi-lumen flexible polymeric extrusion assembly (427) defining a containment structure or cage, each leaf member having an eyelet containing tip (330), a width and a thickness as well as multi-lumen flexible polymeric extrusion assembly having eyelets (533a, 533b) disposed at distal end, confinement sleeve tip (264) and tip component (266) that cooperate to provide a guidance assembly fixed at the forward end (27) of delivery cannula (22) and configured to effect deployment of the leaf members and multi-lumen flexible polymeric extrusion assembly into tissue at an angle of attack, the leaf member and multi-lumen flexible polymeric extrusion assembly widths and thicknesses of an extent effecting formation of a generally curvilinear cage periphery when forward portion of tissue cutting and capture assembly (329) is subsequently extended while eyelet containing tip of each leaf member and multi-lumen flexible polymeric extrusion assembly of tissue cutting and capture assembly is drawn mutually inwardly toward the longitudinal axis (8) to define a curvilinear profile and to close the leading edge of the tissue cutting and capture assembly about a targeted tissue volume (354).

12. The apparatus of claim 11, wherein each leaf member (82-86) is formed of metal; and each leaf member includes an electrically insulative coating having a thickness in a range of about 0.00015 to about 0.0005 inch.

13. The apparatus of claim 12, wherein each leaf member (82-86) width and multi-lumen flexible polymeric extrusion assembly width are of an extent effective to provide extensional cage defining stable movement of the leaf members and multi-lumen flexible polymeric extrusion assembly through the guidance assembly along a plane extending through the longitudinal axis.

14. The apparatus of claim 13, wherein the eyelet containing tip (330) of each leaf member (82-86) and electrically and thermally conductive eyelets (533a, 533b) at distal end of multi-lumen flexible polymeric extrusion assembly incorporate an eyelet aperture dimensioned to receive the first through sixth segments of the resistively heated portion of the of the cutting and pursing cable (89-94) and enable its sliding movement through the eyelets (327a-327e) disposed at the distal ends of first through fifth leaf members (82-86) as well as sliding movement through the first and second electrically and thermally conductive eyelets (533a, 533b) disposed at the distal end of multi-lumen flexible polymeric extrusion assembly (427);
the first segment of the resistively heated portion of the cutting and pursing cable (89) extending from first electrically and thermally conductive eyelet (533a) and through the eyelet (327a) at the eyelet containing distal end of adjacent leaf member (82);
the sixth segment of the resistively heated portion of the cutting and pursing cable (94) extending from second electrically and thermally conductive eyelet (533b) and through the eyelet (327e) at the eyelet containing distal end of adjacent leaf member (86);
the cutting and pursing cable (33) functioning in two distinctly different modes depending on whether the cutting and pursing cable is proximal or distal to its points of electrical contact (534a, 534b) at the distal ends of first and second electrically and thermally conductive eyelets (533a, 533b);
those portions of the cutting and pursing cable (33) that are proximal to the points of electrical contact (534a, 534b) at the distal ends of first and second electrically and thermally conductive eyelets (533a, 533b) having no electrical current flow and are referred to as the first and second tensionable portions (118, 119) of the cutting and pursing cable;
those portions of the cutting and pursing cable (33) that are distal to the points of electrical contact (534a, 534b) that enable electric current to commence to flow from first and second electrically and thermally conductive eyelets (533a and 533b) into the resistively heated portion of the cutting and pursing cable;
a current flow path (399) continuing along the length of electrically and thermally conductive wire (518) until electrical current commences to flow at point of electrical contact (534a) forming a transition boundary for current flow between first electrically and thermally conductive eyelet (533a) and first segment of the resistively heated portion of cutting and pursing cable (89);
the current flow path (399) continuing in first through sixth segments of resistively heated portion of the cutting and pursing cable (89 through 94) until it reaches electrically and thermally conductive eyelet (533b) at the point of electrical contact (534b) at the distal end of the multi-lumen flexible polymeric extrusion assembly (427) forming a transition boundary for current flow between the sixth segment of the resistively heated portion of cutting and pursing cable (94) and the second electrically and thermally conductive eyelet (533b); and
the current flow path (399) continuing along the length of second electrically and thermally conductive wire (520).

15. The apparatus of claim 1 wherein the material for the leaf members (82-86) is austenitic stainless steel.

16. The apparatus of claim 1 wherein the width, W₄ of the leaf members (82-86) is 0.060 to 0.090 inch.

17. The apparatus of claim 1 wherein the thickness, t₁ of the leaf members (82-86) is 0.003 to 0.007 inch.

18. The apparatus of claim 1 wherein the material for the leaf members (82-86) is full-hard austenitic stainless steel.

19. The apparatus of claim 1 wherein the material for the multi-lumen flexible polymeric extrusion (420 or 508) is a synthetic extrudable and biocompatible polymer comprising a polyamide, a polyethylene fluorinated ethylene propylene, or a polytetrafluoroethylene.

20. The apparatus of claim 1 wherein the height, H₂ of the multi-lumen flexible polymeric extrusion (420 or 508) is 0.020 to 0.035 inch.

21. The apparatus of claim 1 wherein the width, W₈ of the multi-lumen flexible polymeric extrusion (420 or 508) is 0.090 to 0.125 inch.

22. The apparatus of claim 1 wherein the leaf member is covered by a biocompatible, electrically insulative coating having a thickness in the range from 0.00015 to 0.00050 inch.

23. The apparatus of claim 22 wherein the biocompatible, electrically insulative coating is capable of operating at temperature of up to 400° C.

24. The apparatus of claim 1, wherein programmed first microcomputer and programmed second microcomputer are replaced by a single programmed microcomputer.

24. A method for the retrieval of a targeted tissue volume (354) of predetermined peripheral extent from human patient, comprising the steps of:
(a) providing a reusable handpiece assembly (15) incorporating a circuit board assembly (184), a constant current source (247), a rechargeable battery (183), practitioner-accessible control switches (39, 40), visible indicators (42, 46, 50), speaker (200) and practitioner-accessible capture size selection switch (479) located on the handpiece assembly for pre-selection of the diameter of a substantially spherical volume of tissue, display of selected capture size (485);
(b) providing a programmed first microcomputer (202a) within circuit board assembly (184) in handpiece assembly responding to signals from practitioner-accessible control switches (39, 40), controlling illumination of visible indicators (42, 46, 50) and controlling activation of audible tone from speaker (200) during period of applying constant current to resistively heated portions of cutting and pursing cable;
(c) providing the programmed first microcomputer (202a) within the circuit board assembly (184) in the handpiece assembly for continuously measuring the electrical impedance of the resistively heated cutting and pursing cable circuit while applying a constant current to the resistively heated portion of the cutting and pursing cable;
(d) providing a second programmed microcomputer (202b) within a circuit board assembly in handpiece assembly interrupting the application of constant current to the resistively heated cutting and pursing cable circuit while continuing to advance the multi-lumen flexible polymeric extrusion and leaf members for period Δₚₐᵤₛₑ if measured increase in the electrical impedance of the resistively heated cutting and pursing cable circuit, Δ_{CR} during a predetermined brief time interval, Δₜ exceeds the maximum acceptable increase, Δₘₐₓ associated with temporary loss of good electrical contact between the first and/or sixth segments of the resistively heated portion of the cutting and pursing cable and the first and/or second electrically and thermally conductive eyelets (533a and/or 533b), respectively;
(e) providing the programmed second microcomputer (202b) within the circuit board assembly in the handpiece assembly for computing the rate of increase, Rᵣₐₜₑ of electrical impedance of the resistively heated cutting and pursing cable circuit as a function of time during the initial period of deployment, tᵣₐₘₚₛₜₐᵣₜ corresponding to initial ramp of deployment of leaf members and extrusion member within tissue capture basket (326) and interrupting application of constant current and voltage supplied to first motor (170a) if rate, Rᵣₐₜₑ is less that a predetermined minimum rate of resistance increase, Rᵣₐₜₑₘᵢₙ;
(f) providing a first motor-actuatable drive tube drive member translation assembly (180a) within handpiece assembly that is in driven engagement with a drive tube drive member (324) within a single-use support housing (100) for advancing a tissue cutting and capture assembly (329) forwardly for retrieving targeted tissue volume;
(g) providing a second motor-actuated cable mounting hub translation assembly (180b) within handpiece assembly that is in driven engagement with a cable mounting hub (296) within a single-use support housing (100) for driving cable mounting hub rearwardly thereby withdrawing the first and second tensioning portions of the cutting and pursing cables (118, 119) attached to the cable mounting hub thereby effecting the pursing down of distal ends of the leaf members (82-86) and multi-lumen flexible polymeric extrusion assembly (427) and forming tissue capture basket (326);
(h) providing a receiving cavity (166) within handpiece assembly for inserting single-use tissue incision and retrieval assembly (12);
(i) inserting a single-use tissue incision and retrieval assembly into receiving cavity (166) within the handpiece assembly, the single-use tissue incision and retrieval assembly incorporating a delivery cannula (22) having an outer surface surmounting an interior channel (263) and extending from a proximal end portion along a longitudinal axis (8) to a forward end (27) having a distal end assembly (25) positionable in confronting adjacency with a targeted tissue volume (354);
(j) administering local anesthetic agent at an intended excision tissue site for excision of a target tissue volume (354) and pausing for a time period sufficient for the injected local anesthetic agent to achieve an anesthetic effect regionally about the intended excision tissue site;
(k) incising a skin site (24) adjacent to a region of intended excision of the target tissue volume with scalpel to a depth of about 2 mm and a width about 2 mm wider than a maximum width of blade (31) at the forward end of the cannula (27);
(l) advancing the forward end of the cannula under guidance of an imagining apparatus comprising an ultrasound apparatus, a radiographic apparatus, or a magnetic resonance apparatus, for positioning the forward end of the cannula just proximal to and in confronting adjacency to the target tissue volume (354);
(m) advancing a leaf member and multi-lumen flexible polymeric extrusion member assembly (400) positioned within the interior channel of the delivery cannula at the forward end of the delivery cannula having a forward portion extending to forwardly disposed first through sixth segments of the resistively heated portion of the cutting and pursing cable (89-94) that are electrical communicating with first and second electrically and thermally conductive eyelets (533a, 533b) located at distal ends of first and second electrically and thermally conductive wires (518, 520), respectively;
(n) applying a predetermined level of constant current to the first through sixth segments of the resistively heated portion of cutting and pursing cable (89-94) supplied by a constant current source (247) incorporated in circuit board assembly (184) while driving the tissue cutting and capture assembly (329) forwardly by applying a predetermined level of voltage to first motor-actuatable drive tube drive member translation assembly (180a);
(o) advancing the distal ends of the leaf members (82-86) and multi-lumen flexible polymeric extrusion assembly (427) through an arcuate path defining the hemispherical shape of the proximal end of the tissue capture basket and continuing to a maximum peripheral extent, then immediately applying a predetermined level of voltage to a second motor-actuated cable mounting hub translation assembly (180b) within the handpiece assembly that is in driven engagement with a cable mounting hub (296) within a single-use support housing (100) thereby driving the cable mounting hub rearwardly while withdrawing the first and second tensioning portions of the cutting and pursing cable (118, 119) attached to the cable mounting hub and effecting pursing down of the distal ends of the leaf members (82-86) and multi-lumen flexible polymeric extrusion assembly forming a tissue capture basket (326); and
(p) removing the captured target tissue volume (354) by withdrawing the cannula (22) along with the tissue cutting and capture assembly.

25. The method of claim 24, wherein the material for the leaf members (82-86) is austenitic stainless steel.

26. The method of claim 24, wherein the width, W₄ of the leaf members (82-86) is 0.060 to 0.090 inch.

27. The method of claim 24, wherein the thickness, t₁ of the leaf members (82-86) is 0.003 to 0.007 inch.

28. The method of claim 24, wherein the material for the leaf members (82-86) is full-hard austenitic stainless steel.

29. The method of claim 24, wherein the material for the multi-lumen flexible polymeric extrusion (420 or 508) is a synthetic extrudable and biocompatible polymer including polyamides (e.g., Nylon 6, Nylon 11 and Nylon 12), polyethylene fluorinated ethylene propylene and polytetrafluoroethylene.

30. The method of claim 24, wherein the height, H₂ of the multi-lumen flexible polymeric extrusion (420 or 508) is 0.020 to 0.035 inch.

31. The method of claim 24, wherein the width, W₈ of the multi-lumen flexible polymeric extrusion (420 or 508) is 0.090 to 0.125 inch.

32. The method of claim 24, wherein the leaf member is covered by a biocompatible, electrically insulative coating having a thickness in the range from 0.00015 to 0.00050 inch.

33. The method of claim 32, wherein the biocompatible, electrically insulative coating is capable of operating at temperature of up to 400° C.

34. The method of claim 24, wherein the rate of advancement of first motor-actuatable drive tube drive member translation assembly (180a) within handpiece assembly (15) is in the range from 2.0 to 3.0 mm/second.

35. The method of claim 24, wherein the cutting and pursing cable (33) comprises multiple stainless steel wires, each wire having a diameter of between about 0.0008 to about 0.002 inch.

36. The method of claim 24, wherein the material of the metal wire is austenitic stainless steel, nickel alloy, cobalt/nickel alloy, titanium or titanium alloy.

37. The method of claim 24, wherein the functions performed in the first programmed microcomputer and the second programmed microcomputer are performed within a single programmed microcomputer.

38. A method for thermal cutting of human tissue at a cutting rate, Rout by a cable (33) having a diameter, D_{cable} that is resistively heated by the passage of substantially constant level of current, I_{cut} wherein the minimum level of substantially constant current delivered to the resistively heated cable to effect thermal cutting of human tissue is determined by the generalized equation:
$${I}_{cut} = {[ \begin{matrix}\begin{matrix}( ( N \times ( 3.1416 / 4 ) \times {( {D}_{wire} )}^{2} ) / {ρ}_{cable} ) \times 4.186 \times \\ {d}_{water} \times ( {D}_{cable} \times 1.2 ) \times {R}_{cut} \times WC \times \end{matrix} \\ ( {C}_{water} \times Δ T + {LH}_{water} )\end{matrix} ]}^{0.5}$$
wherein N is the number of wires in cable, D_{wire} is the diameter of the wire within the cable (in units of cm);
ρ_{cable} is the electrical resistivity of the wires within the cable (in units of ohm-cm);
d_{water} is the density of the cellular water (in units of grams/cm³);
D_{cable} is the overall diameter of the cable (in units of cm);
D_{wire} is the diameter of each wire within the cable (33) (in units of cm);
R_{cut} is the rate of cutting within tissue (in units of cm/sec);
WC is a unitless fractional value corresponding to the water content of tissue per unit mass of tissue;
C_{water} is the specific heat of cellular water (in units of calories/gram-° C.);
LH_{water} is the latent heat of vaporization of cellular water (in units of calories/gram); and
ΔT is the required temperature rise of cellular water at normal body temperature of 37° C. to the boiling point of water of 100° C.

39. A method for thermal cutting of human tissue at a cutting rate, Rout by a cable (33) having a diameter, D_{cable} that is electrically resistively heated by the passage of a substantially constant level of current, I_{cut} wherein the minimum heat flux dissipated at the surface of the resistively heated cable to affect the thermal cutting of human tissue is represented by a generalized equation:
$${P}_{cut} / {A}_{surface} = ( 1 / ( 3.1416 \times {D}_{cable} ) ) \times 4.186 \times {d}_{water} \times ( {D}_{cable} \times 1.2 ) \times {R}_{cut} \times WC \times ( {C}_{water} \times Δ T + {LH}_{water} ) ,$$
wherein D_{cable} is the overall diameter of the cable (in units of cm);
d_{water} is the density of the cellular water (in units of grams/cm³);
R_{cut} is the rate of cutting within tissue (in units of cm/see);
WC is a unitless fractional value corresponding to the water content of tissue per unit mass of tissue;
C_{water} is the specific heat of cellular water (in units of calories/gram-° C.);
LH_{water} is the latent heat of vaporization of cellular water (in units of calories/gram); and
ΔT is the required temperature rise of cellular water at normal body temperature of 37° C. to the boiling point of water of 100° C.

40. An apparatus for the thermal cutting of human tissue, comprising:
(a) a cable (33) having a diameter, D_{cable}; and
(b) a current generator that generates a substantially constant level of current and which current generator is in electrical connection with the cable for passage of a substantially constant level of current, I_{cut} for resistively heating the cable for thermally cutting human tissue at a cutting rate, Rout wherein the minimum level of substantially constant current delivered to the resistively heated cable by the current generator to effect the thermal cutting of human tissue is determined by the generalized equation:
$${I}_{cut} = {[ \begin{matrix}\begin{matrix}( ( N \times ( 3.1416 / 4 ) \times {( {D}_{wire} )}^{2} ) / {ρ}_{cable} ) \times 4.186 \times \\ {d}_{water} \times ( {D}_{cable} \times 1.2 ) \times {R}_{cut} \times WC \times \end{matrix} \\ ( {C}_{water} \times Δ T + {LH}_{water} )\end{matrix} ]}^{0.5}$$
where N is the number of wires in cable, D_{wire} is the diameter of the wire within the cable (in units of cm);
ρ_{cable} is the electrical resistivity of the wires within the cable (in units of ohm-cm);
d_{water} is the density of the cellular water (in units of grams/cm³);
D_{cable} is the overall diameter of the cable (in units of cm);
D_{wire} is the diameter of each wire within the cable (33) (in units of cm);
R_{cut} is the rate of cutting within tissue (in units of cm/sec);
WC is a unitless fractional value corresponding to the water content of tissue per unit mass of tissue;
C_{water} is the specific heat of cellular water (in units of calories/gram-° C.);
LH_{water} is the latent heat of vaporization of cellular water (in units of calories/gram); and
ΔT is the required temperature rise of cellular water at normal body temperature of 37° C. to the boiling point of water of 100° C.

41. The apparatus of claim 40 wherein the diameter of the individual wires, D_{wire} within cable (33) is in the range from 0.0010 inch to 0.0015 inch (0.0025 cm to 0.0038 cm) and the number of wires, N in the cable is 7 to 19.

42. The apparatus of claim 40 wherein the diameter of the cable (33), D_{cable} is in the range from 0.0030 inch to 0.0075 inch (0.0076 cm to 0.0191 cm)

43. The apparatus of claim 40 wherein the individual wires are comprised of a cobalt/chrome/tungsten/nickel alloy such as Hastalloy C alloy, also known as L605 alloy.

44. The method of claim 41 wherein the rate of cutting, Rout within tissue is in the range from 0.2 to 0.3 (in units of cm/sec).

45. An apparatus for the thermal cutting of human tissue, comprising:
(a) a cable (33) having a diameter, D_{cable}; and
(b) a current generator that generates a substantially constant level of current and which current generator is in electrical connection with the cable for passage of a substantially constant level of current, I_{cut} for resistively heating the cable for thermally cutting human tissue at a cutting rate, Rout a minimum heat flux dissipated at the surface of the resistively heated cable to affect the thermal cutting of human tissue is represented by a generalized equation:
$${P}_{cut} / {A}_{surface} = ( 1 / ( 3.1416 \times {D}_{cable} ) ) \times 4.186 \times {d}_{water} \times ( {D}_{cable} \times 1.2 ) \times {R}_{cut} \times WC \times ( {C}_{water} \times Δ T + {LH}_{water} ) ,$$
wherein D_{cable} is the overall diameter of the cable (in units of cm);
d_{water} is the density of the cellular water (in units of grams/cm³);
R_{cut} is the rate of cutting within tissue (in units of cm/sec);
WC is a unitless fractional value corresponding to the water content of tissue per unit mass of tissue;
C_{water} is the specific heat of cellular water (in units of calories/gram-° C.);
LH_{water} is the latent heat of vaporization of cellular water (in units of calories/gram); and
ΔT is the required temperature rise of cellular water at normal body temperature of 37° C. to the boiling point of water of 100° C.
